SEQUENCE LISTING (33 sequences)

SEQ ID NO: 1 - DNA, 48 bp, Artificial Sequence
  Synthetic Polynucleotide
ttaacccacgccgaatcctagactcaaagtagtctaggattcggcgtg

SEQ ID NO: 2 - DNA, 48 bp, Artificial Sequence
  Synthetic Polynucleotide
agtctaggattcggcgtgggttaacacgccgaatcctagactactttg

SEQ ID NO: 3 - DNA, 48 bp, Artificial Sequence
  Synthetic Polynucleotide
ttaacccacgccgaatcctagactcaaagtagtctaggattcggcgag

SEQ ID NO: 4 - DNA, 48 bp, Artificial Sequence
  Synthetic Polynucleotide
actctaggattcggcgtgggttaacacgccgaatcctagactactttg

SEQ ID NO: 5 - DNA, 48 bp, Artificial Sequence
  Synthetic Polynucleotide
ttaacccacgccgaatcctagactcaaagtagtctaggattcggggtg

SEQ ID NO: 6 - DNA, 48 bp, Artificial Sequence
  Synthetic Polynucleotide
agtctaggattcggcgtgggttaacacgccgaatcctacactactttg

SEQ ID NO: 7 - DNA, 48 bp, Artificial Sequence
  Synthetic Polynucleotide
ttaacccacgccgaatcctagactcaaagtagtctaggattccgcgtg

SEQ ID NO: 8 - DNA, 48 bp, Artificial Sequence
  Synthetic Polynucleotide
agtcttggattcggcgtgggttaacacgccgaatcctagactactttg

SEQ ID NO: 9 - DNA, 48 bp, Artificial Sequence
  Synthetic Polynucleotide
ttaacccacgccgaatcctagactcaaagtagtctaggatacggcgtg

SEQ ID NO: 10 - DNA, 48 bp, Artificial Sequence
  Synthetic Polynucleotide
agtctagcattcggcgtgggttaacacgccgaatcctagactactttg

SEQ ID NO: 11 - DNA, 24 bp, Artificial Sequence
  Synthetic Polynucleotide
agtctaggattcggcgtgggttaa

SEQ ID NO: 12 - DNA, 24 bp, Artificial Sequence
  Synthetic Polynucleotide
caaagtagtctaggattcggcgtg

SEQ ID NO: 13 - DNA, 24 bp, Artificial Sequence
  Synthetic Polynucleotide
caaagtagtgtaggattcggcgtg

SEQ ID NO: 14 - DNA, 24 bp, Artificial Sequence
  Synthetic Polynucleotide
caaagtactctaggattcggcgtg

SEQ ID NO: 15 - DNA, 48 bp, Artificial Sequence
  Synthetic Polynucleotide
agtctaggattcggcgtgggttaacacgccgaatcctagagtactttg

SEQ ID NO: 16 - DNA, 48 bp, Artificial Sequence; identical to SEQ ID NO: 5
  Synthetic Polynucleotide

SEQ ID NO: 17 - DNA, 48 bp, Artificial Sequence; identical to SEQ ID NO: 7
  Synthetic Polynucleotide

SEQ ID NO: 18 - DNA, 24 bp, Artificial Sequence
  Synthetic Polynucleotide
caaagtagtcttggattcggcgtg

SEQ ID NO: 19 - DNA, 48 bp, Artificial Sequence
  Synthetic Polynucleotide
agtctaggattcggcgtgggttaacacgccgaatccaagactactttg

SEQ ID NO: 20 - DNA, 24 bp, Artificial Sequence
  Synthetic Polynucleotide
caaagtagtctagcattcggcgtg

SEQ ID NO: 21 - DNA, 48 bp, Artificial Sequence
  Synthetic Polynucleotide
agtctaggattcggcgtgggttaacacgccgaatgctagactactttg

SEQ ID NO: 22 - DNA, 24 bp, Artificial Sequence
  Synthetic Polynucleotide
  misc_feature (1)..(1): BHQ
agtctaggattcggcgtgggttaa

SEQ ID NO: 23 - DNA, 47 bp, Artificial Sequence
  Synthetic Polynucleotide
  misc_feature (25)..(25): CY3
ttaacccacgccgaatcctagactaaagtagtctaggattcggcgtg

SEQ ID NO: 24 - DNA, 24 bp, Artificial Sequence
  Synthetic Polynucleotide
  misc_feature (24)..(24): BHQ
caaagtagtgtaggattcggcgtg

SEQ ID NO: 25 - DNA, 24 bp, Artificial Sequence
  Synthetic Polynucleotide
  misc_feature (24)..(24): BHQ
caaagtagtctaggattcggggtg

SEQ ID NO: 26 - DNA, 47 bp, Artificial Sequence
  Synthetic Polynucleotide
  misc_feature (24)..(24): CY3
agtctaggattcggcgtgggttacacgccgaatcctacactactttg

SEQ ID NO: 27 - DNA, 12 bp, Artificial Sequence
  Synthetic Polynucleotide
tacactactttg

SEQ ID NO: 28 - DNA, 12 bp, Artificial Sequence
  Synthetic Polynucleotide
tacactactttg

SEQ ID NO: 29 - DNA, 12 bp, Artificial Sequence
  Synthetic Polynucleotide
caaagtagtgta

SEQ ID NO: 30 - DNA, 12 bp, Artificial Sequence
  Synthetic Polynucleotide
gccgaatcctac

SEQ ID NO: 31 - DNA, 12 bp, Artificial Sequence
  Synthetic Polynucleotide
ctaggattcggg

SEQ ID NO: 32 - DNA, 20 bp, Artificial Sequence
  Synthetic Polynucleotide
ttaacccacgccgcggggtg

SEQ ID NO: 33 - DNA, 20 bp, Artificial Sequence
  Synthetic Polynucleotide
gtttcatcacatcgatctga

(19) United States
(12) Patent Application Publication
(10) Pub. No.: US 2022/0349005 A1
(43) Pub. Date: Nov. 3, 2022

(54) CONTROLLING THE DNA HYBRIDIZATION CHAIN REACTION

(71) Applicant: NORTHWESTERN UNIVERSITY, Evanston, IL (US)

(72) Inventors: Chad A. Mirkin, Wilmette, IL (US); Charles A. Figg, Evanston, IL (US)

(21) Appl. No.: 17/634,908

(22) PCT Filed: Aug. 19, 2020
(86) PCT No.: PCT/US2020/047040

§ 371 Date: Feb. 11, 2022

Related U.S. Application Data
(60) Provisional application No. 62/888,785, filed on Aug. 19, 2019.

(51) Int. Cl. C12Q 1/6876

(52) U.S. Cl. CPC C12Q 1/6876; C12Q 1/6818

(57) ABSTRACT

The present disclosure is generally directed to methods for controlling the polymerization of metastable oligonucleotide hairpins using the hybridization chain reaction (HCR) by introducing one or more base pair mismatches in the hairpins. Control was achieved through the introduction of a base-pair mismatch in the duplex of the hairpins. The mismatch modification allows one to energetically differentiate initiation versus propagation events, leading to DNA oligomers up to 10-mers with degree of polymerization (DP) dispersity between 1.3 and 1.6.

### CROSS-REFERENCE TO RELATED APPLICATIONS

This application claims the priority benefit under 35 U.S.C. § 119(e) of U.S. Provisional Patent Application No. 62/888,785, filed Aug. 19, 2019, which is incorporated herein by reference in its entirety.

### STATEMENT OF GOVERNMENT INTEREST

This invention was made with government support under N00014-15-1-0043 awarded by the Office of Naval Research (ONR). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2019-048_Seqlisting.txt”, which was created on Aug. 19, 2020 and is 7,054 bytes in size. The subject matter of the Sequence Listing is incorporated herein in its entirety by reference.

### FIELD

The present disclosure is directed to methods for controlling the polymerization of metastable DNA hairpins using the hybridization chain reaction (HCR).

### BACKGROUND

DNA oligomers are programmable nanoscale scaffolds that are synthesized through numerous approaches [Wang et al., Curr. Opin. Bio-technol. 2013, 24, 562-574; Jones et al., Science 2015, 347 1260901; Hong et al., Chem. Rev. 2017, 117, 12584-12640; McMillan et al., J. Am. Chem. Soc. 2018, 140, 6776-6779; Kashiwagi et al., J. Am. Chem. Soc. 2018, 140, 26-29; Laramy et al., Nat. Rev. Mater. 2019, 4, 201-224]. However, approaches that avoid extensive sequence design usually result in high-dispersity DNA oligomers [Ali et al., Chem. Soc. Rev. 2014, 43, 3324-3341; Zhao et al., Chem. Rev. 2015, 115, 12491-12545.], and approaches that yield well-defined oligomers generally require extensive sequence design, sequential monomer addition, and/or purification [Hamblin et al., Nat. Commun. 2015, 6, 7065; Pound et al., Nano Lett. 2009, 9, 4302-4305; Greschner et al., Biomacromolecules 2014, 15, 3002-3008].

DNA polymers are programmable nanoscale scaffolds.¹ One DNA polymerization approach, the hybridization chain reaction (HCR),² mimics molecular chain-growth polymerization. In this system, the secondary structure of DNA is utilized to increase the energy barrier associated with polymerization. Two DNA hairpins containing complementary regions are kinetically trapped until the introduction of an initiator strand triggers a chain reaction of DNA duplexing via sequential strand displacement reactions. Temporal control and DNA sequence recognition in HCR have led to extensive monomer design³⁻⁵ and application in cases where base pairing drives maximum signal output (e.g., mRNA expression levels,^{6,7} small molecule detection, molecular machines^{8,9}). Additionally, HCR has recently been used to polymerize functional materials, such as responsive DNA sequences, metallic nanoparticles,¹⁰ synthetic polymers,¹¹ or proteins.¹² However, DNA polymer length is empirically determined, prohibiting predictable reactions where initiator (e.g., mRNA) to monomer ratios result in a polymerization output (e.g., fluorescence) that can be quantitatively correlated to reaction stoichiometry.

### SUMMARY

The present disclosure provides methods for controlling the polymerization of metastable DNA hairpins using the hybridization chain reaction (HCR). Control was achieved through the introduction of a base-pair mismatch in the duplex of the hairpins. The mismatch modification allows one to energetically differentiate initiation versus propagation events, leading to DNA oligomers up to 10-mers with degree of polymerization (DP) dispersity between 1.3 and 1.6. Importantly, even after two consecutive chain extensions from DP=2 to a final DP=14, dispersity remains unaffected, showing that well-defined block copolymers can be achieved. As a proof-of-concept, this technique was applied to hairpin monomers functionalized with a mutant green fluorescent protein to prepare well-defined protein polymers. Taken together, the disclosure defines an effective method for controlling HCR polymerization with macromolecules in a manner analogous to the controlled polymerization of small molecules.

Applications of the methods of the disclosure include, but are not limited to:
Nucleotide detection (mRNA, anti-sense oligonucleotides, DNA, etc.)
Analyte detection (e.g., using DNA aptamers)
Responsive DNA-based materials
Protein assembly for emergent or synergistic reactivity (e.g., enzyme cascade reactions, multivalent antibody recognition, co-delivery of therapeutic proteins)
Plasmonic, catalytic, semiconductor nanoparticle assemblies

Advantages provided by the disclosure include but are not limited to:
Quantitative relationship between monomer, initiator, and oligomer
Predictable molecular weight and low dispersity DNA oligomers
DNA block co-oligomers with predictable molecular weights and quantitative chain extensions.

In some aspects, the disclosure provides a method of producing a structure, the method comprising contacting a) a first oligonucleotide hairpin monomer comprising a first toehold, a first duplex stem, and a first hairpin loop, wherein the first duplex stem comprises a base-pair mismatch at a position that is proximal to the first toehold and distal to the first hairpin loop, relative to a midpoint of the first duplex stem; b) a second oligonucleotide hairpin monomer comprising a second toehold, a second duplex stem, and a second hairpin loop; and c) an initiator oligonucleotide, wherein hybridization of the initiator oligonucleotide to the first oligonucleotide hairpin monomer results in hybridization of the first oligonucleotide hairpin monomer to the second oligonucleotide hairpin monomer, thereby producing the structure. In some embodiments, the base-pair mismatch in the first duplex stem is located at a position that is 1, 2, 3, 4, 5, 6, 7, 8, 9, 10, 11, 12, 13, 14, or 15 nucleotides from the first toehold. In further embodiments, the first duplex stem comprises 2, 3, 4, or 5 base-pair mismatches. In some embodiments, the second duplex stem comprises a base-pair mismatch at a position that is proximal to the second toehold and distal to the second hairpin loop, relative to a midpoint of the second duplex stem. In some embodiments, the base-pair mismatch in the second duplex stem is located at a position that is 1, 2, 3, 4, 5, 6, 7, 8, 9, 10, 11, 12, 13, 14, or 15 nucleotides from the second toehold. In further embodiments, the second duplex stem comprises 2, 3, 4, or 5 base-pair mismatches. In some embodiments, the first duplex stem comprises from about 10 to about 50 nucleotides. In some embodiments, the second duplex stem comprises from about 10 to about 50 nucleotides. In further embodiments, the first hairpin loop comprises from about 3 to about 30 nucleotides. In some embodiments, the second hairpin loop comprises from about 3 to about 30 nucleotides. In some embodiments, the toehold of the first oligonucleotide hairpin monomer comprises from about 1 to about 50 nucleotides. In some embodiments, the toehold of the second oligonucleotide hairpin monomer comprises from about 1 to about 50 nucleotides. In some embodiments, the second hairpin loop and the first toehold are about 70%, about 80%, about 90%, about 99%, or 100% complementary to each other. In further embodiments, the second toehold and the first hairpin loop are about 70%, about 80%, about 90%, about 99%, or 100% complementary to each other. In some embodiments, the initiator oligonucleotide is an analyte to be detected in a sample. In further embodiments, the initiator oligonucleotide is single stranded. In some embodiments, the initiator oligonucleotide is RNA. In still further embodiments, the initiator oligonucleotide is messenger RNA (mRNA). In some embodiments, the first hairpin oligonucleotide monomer further comprises an additional moiety. In some embodiments, the second oligonucleotide hairpin monomer further comprises an additional moiety. In some embodiments, the initiator oligonucleotide further comprises an additional moiety. In further embodiments, the additional moiety is an aptamer, protein, a peptide, a nanoparticle, a small molecule a quantum dot, a detectable marker, or a combination thereof. In some embodiments, the protein is an antibody. In some embodiments, the first oligonucleotide hairpin monomer further comprises an antibody and the second oligonucleotide hairpin monomer further comprises an additional antibody. In further embodiments, the antibody and the additional antibody are different. In some embodiments, the detectable marker is a fluorophore. In some embodiments, a plurality of first oligonucleotide hairpin monomers, initiator oligonucleotides, and second oligonucleotide hairpin monomers are contacted. In some embodiments, the ratio of the first oligonucleotide hairpin monomer to the initiator oligonucleotide is about 1:1, 2:1, 4:1, 6:1, 8:1, or 10:1. In some embodiments, the ratio of the second oligonucleotide hairpin monomer to the initiator oligonucleotide is about 1:1, 2:1, 4:1, 6:1, 8:1, or 10:1. In further embodiments, the structure is detected by gel electrophoresis, mass spectrometry, light scattering spectroscopy, colorimetry, fluorescent microscopy, fluorescent spectroscopy, electron microscopy, atomic force microscopy, nuclear magnetic resonance (NMR) depending or a combination thereof. In some embodiments, the first oligonucleotide hairpin monomer is DNA, RNA, or a modified form thereof. In further embodiments, the second oligonucleotide hairpin monomer is DNA, RNA, or a modified form thereof. In some embodiments, the initiator oligonucleotide is DNA, RNA, or a modified form thereof. In some embodiments, methods of the disclosure further comprise contacting the structure with a termination oligonucleotide.

In some aspects, the disclosure provides a method of detecting an analyte in a sample, the method comprising contacting the sample with a) a first oligonucleotide hairpin monomer comprising a first toehold, a first duplex stem, a first hairpin loop, and a fluorescent marker, wherein the first duplex stem comprises a base-pair mismatch at a position that is proximal to the first toehold and distal to the first hairpin loop, relative to a midpoint of the first duplex stem; b) an initiator oligonucleotide; and c) a second oligonucleotide hairpin monomer comprising a second toehold, a second duplex stem, and a second hairpin loop; wherein one or more of the first duplex stem, the first toehold, and the initiator oligonucleotide comprises an analyte-binding region; and measuring fluorescence of the sample after the contacting to detect the analyte. In some embodiments, the analyte is a nucleic acid. In some embodiments, the analyte is DNA, RNA, a protein, a peptide, or a combination thereof. In further embodiments, the analyte is messenger RNA (mRNA), microRNA (miRNA), small interfering RNA (siRNA), or a combination thereof. In some embodiments, the analyte-binding region is substantially complementary to the nucleic acid. In some embodiments, the second oligonucleotide hairpin monomer comprises an additional fluorescent marker. In some embodiments, the fluorescent marker and the additional fluorescent marker are different. In some embodiments, the analyte-binding region comprises an antibody. In some embodiments, the sample is contacted with a plurality of first oligonucleotide hairpin monomers, initiator oligonucleotides, and/or second oligonucleotide hairpin monomers.

In further aspects, the disclosure provides a method of producing a structure comprising hybridized oligonucleotide monomers, the method comprising contacting: a) a first oligonucleotide hairpin monomer comprising: (i) a first duplex stem comprising nucleotide sequence B and nucleotide sequence C that are substantially complementary to each other, (ii) a first hairpin loop at a first end of the first duplex stem, and (iii) a toehold comprising nucleotide sequence A at a second end of the first duplex stem, wherein the first duplex stem comprises a base-pair mismatch at a position that is proximal to nucleotide sequence A and distal to the first hairpin loop, relative to a midpoint of the first duplex stem; b) an initiator oligonucleotide comprising: (i) nucleotide sequence D that is substantially complementary to nucleotide sequence A of the first oligonucleotide hairpin monomer, and (ii) nucleotide sequence E that is substantially complementary to nucleotide sequence B of the first oligonucleotide hairpin monomer; and c) a second oligonucleotide hairpin monomer comprising: (i) a second duplex stem comprising nucleotide sequence G and nucleotide sequence H that are substantially complementary to each other, (ii) a second hairpin loop at a first end of the second duplex stem that is substantially complementary to nucleotide sequence A of the first oligonucleotide hairpin monomer, and (iii) a toehold comprising nucleotide sequence F at a second end of the second duplex stem that is substantially complementary to the first hairpin loop of the first oligonucleotide hairpin monomer, wherein hybridization of the initiator oligonucleotide to the first oligonucleotide hairpin monomer results in hybridization of the first oligonucleotide hairpin monomer to the second oligonucleotide hairpin monomer via a chain reaction, thereby producing the structure. In some embodiments, the base-pair mismatch in the first duplex stem is located at a position that is 1, 2, 3, 4, 5, 6, 7, 8, 9, 10, 11, 12, 13, 14, or 15 nucleotides from the second end of the first duplex stem. In further embodiments, the first duplex stem comprises 2, 3, 4, or 5 base-pair mismatches. In some embodiments, the second duplex stem comprises a base-pair mismatch at a position that is proximal to nucleotide sequence F and distal to the second hairpin loop, relative to a midpoint of the second duplex stem. In some embodiments, the base-pair mismatch in the second duplex stem is located at a position that is 1, 2, 3, 4, 5, 6, 7, 8, 9, 10, 11, 12, 13, 14, or 15 nucleotides from the second end of the second duplex stem. In further embodiments, the second duplex stem comprises 2, 3, 4, or 5 base-pair mismatches. In some embodiments, the first duplex stem comprises from about 10 to about 50 nucleotides. In some embodiments, the second duplex stem comprises from about 10 to about 50 nucleotides. In some embodiments, the first hairpin loop comprises from about 3 to about 30 nucleotides. In some embodiments, the second hairpin loop comprises from about 3 to about 30 nucleotides. In further embodiments, the toehold of the first oligonucleotide hairpin monomer comprises from about 1 to about 50 nucleotides. In still further embodiments, the toehold of the second oligonucleotide hairpin monomer comprises from about 1 to about 50 nucleotides. In some embodiments, nucleotide sequence D comprises from about 1 to about 20 nucleotides. In some embodiments, nucleotide sequence E comprises from about 10 to about 50 nucleotides. In some embodiments, nucleotide sequence B and nucleotide sequence C are about 70%, about 80%, about 90%, or about 99% complementary to each other. In some embodiments, nucleotide sequence D and nucleotide sequence A are about 70%, about 80%, about 90%, about 99%, or 100% complementary to each other. In further embodiments, nucleotide sequence E and nucleotide sequence B are about 70%, about 80%, about 90%, about 99%, or 100% complementary to each other. In some embodiments, nucleotide sequence G and nucleotide sequence H are about 70%, about 80%, about 90%, about 99%, or 100% complementary to each other. In some embodiments, the second hairpin loop and nucleotide sequence A are about 70%, about 80%, about 90%, about 99%, or 100% complementary to each other. In further embodiments, nucleotide sequence F and the first hairpin loop are about 70%, about 80%, about 90%, about 99%, or 100% complementary to each other. In some embodiments, the initiator oligonucleotide is an analyte to be detected in a sample. In further embodiments, the initiator oligonucleotide is single stranded. In some embodiments, the initiator oligonucleotide is RNA. In further embodiments, the initiator oligonucleotide is messenger RNA (mRNA). In some embodiments, the first hairpin oligonucleotide monomer further comprises an additional moiety. In some embodiments, the second oligonucleotide hairpin monomer further comprises an additional moiety. In some embodiments, the initiator oligonucleotide further comprises an additional moiety. In further embodiments, the additional moiety is an aptamer, protein, a nanoparticle, a quantum dot, a detectable marker, or a combination thereof. In still further embodiments, the protein is an antibody. In some embodiments, the first hairpin oligonucleotide monomer further comprises an antibody and the second oligonucleotide hairpin monomer further comprises an additional antibody. In some embodiments, the antibody and the additional antibody are different. In some embodiments, the detectable marker is a fluorophore. In some embodiments, a plurality of first oligonucleotide hairpin monomers, initiator oligonucleotides, and second oligonucleotide hairpin monomers are contacted. In some embodiments, the ratio of the first oligonucleotide hairpin monomer to the initiator oligonucleotide is about 1:1, 2:1, 4:1, 6:1, 8:1, or 10:1. In some embodiments, the ratio of the second oligonucleotide hairpin monomer to the initiator oligonucleotide is about 1:1, 2:1, 4:1, 6:1, 8:1, or 10:1. In some embodiments, the structure is detected by gel electrophoresis, mass spectrometry, light scattering spectroscopy, colorimetry, fluorescent microscopy, fluorescent spectroscopy, electron microscopy, atomic force microscopy, nuclear magnetic resonance (NMR) depending or a combination thereof. In some embodiments, methods of the disclosure further comprise contacting the structure with a termination oligonucleotide.

In some aspects, the disclosure provides a method of detecting an analyte in a sample, the method comprising contacting the sample with: a) a first oligonucleotide hairpin monomer comprising: (i) a first duplex stem comprising nucleotide sequence B and nucleotide sequence C that are substantially complementary to each other, (ii) a first hairpin loop at a first end of the first duplex stem, (iii) a toehold comprising nucleotide sequence A at a second end of the first duplex stem, and (iv) a fluorescent marker, wherein the first duplex stem comprises a base-pair mismatch at a position that is proximal to nucleotide sequence A and distal to the first hairpin loop, relative to a midpoint of the first duplex stem; b) an initiator oligonucleotide comprising nucleotide sequence E that is substantially complementary to nucleotide sequence B of the first oligonucleotide hairpin monomer; and c) a second oligonucleotide hairpin monomer comprising: (i) a second duplex stem comprising nucleotide sequence G and nucleotide sequence H that are substantially complementary to each other, (ii) a second hairpin loop at a first end of the second duplex stem that is substantially complementary to nucleotide sequence A of the first oligonucleotide hairpin monomer, and (iii) a toehold comprising nucleotide sequence F at a second end of the second duplex stem that is substantially complementary to the first hairpin loop of the first oligonucleotide hairpin monomer, wherein one or more of the initiator oligonucleotide, nucleotide sequence B, and nucleotide sequence A comprise an analyte-binding region; and measuring fluorescence of the sample after the contacting to detect the analyte. In some embodiments, the analyte is a nucleic acid. In some embodiments, the analyte is DNA, RNA, a protein, or a combination thereof. In further embodiments, the analyte is mRNA. In some embodiments, the analyte-binding region is substantially complementary to the nucleic acid. In some embodiments, the second oligonucleotide hairpin monomer comprises an additional fluorescent marker. In some embodiments, the fluorescent marker and the additional fluorescent marker are different. In some embodiments, the analyte-binding region comprises an antibody. In some embodiments, the sample is contacted with a plurality of first oligonucleotide hairpin monomers, initiator oligonucleotides, and/or second oligonucleotide hairpin monomers.

### BRIEF DESCRIPTION OF THE FIGURES

FIG. 1 shows a comparison of (a) previous hybridization chain reaction (HCR) polymerizations where initiation from the intruding strand (I1) and propagation of hairpin 1 (H1) and hairpin 2 (H2) are energetically similar, to (b) the reported HCR polymerization where a mismatch is included to increase the rate of initiation of the intruding strand (I2m) versus propagation of mismatch hairpin 1 (H1m) and mismatch hairpin 2 (H2m).

FIG. 2 shows results of experiments targeting different degrees of polymerization (DP) at varying solution ionic strength. (a) Scheme of initiating mismatch hairpin 2 (H2m) and mismatch hairpin 1 (H1m) with initiator (I2m), (b) agarose gel electrophoresis (1% w/w gel) showing polymer distributions according to target DP (37° C., 16 hours, PBS+1 M NaCl, L1—TrackIt Ultra-Low-Range ladder, L2—GeneRuler 50 bp DNA ladder), (c) Plot of resultant DPs and (d) dispersity according to varying [monomer]:[initiator] compared to theoretical values using PBS with different ionic strengths obtained from densiometry of agarose gels (16 h, PBS reactions were performed at 22 C, PBS+0.5 M NaCl and PBS+1 M NaCl were performed at 37° C.).

FIG. 3 shows consecutive chain extensions of DNA polymers. (a) Scheme showing chain extensions and polymerization conditions, (b) agarose gel electrophoresis (1% w/w) of polymer block 1 (1) and subsequent chain extensions to a second (2, 2′) and third block (3, 3′).

FIG. 4 shows melting curves of DNA hairpin 1 (a) and hairpin 2 (b) in PBS with different mismatches showing the change in melting temperature according to mismatch position.

FIG. 5 shows agarose gel electrophoresis (3 w/w %) of reactions of I+H1+H2 testing the effect of mismatch position on product distribution. Reactions conducted at 5 μM per sequence for 4 hours at 22° C. in PBS.

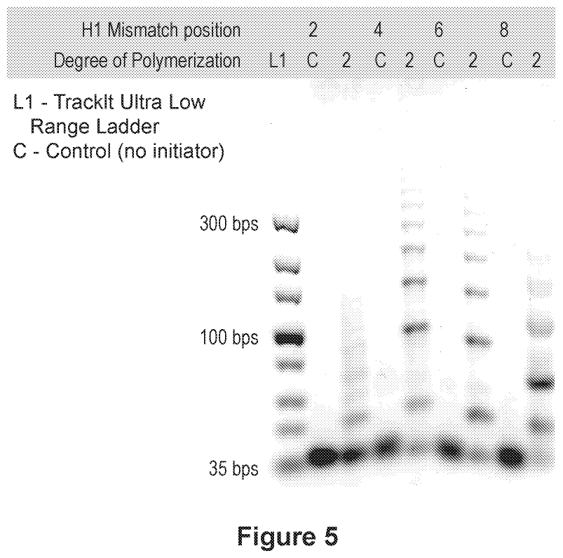

FIG. 6 shows agarose gel electrophoresis (3 w/w %) of reactions of I2B, H1, and H2 testing the effect of mismatch position on product distribution. Reactions conducted at 5 μM per sequence for 4 hours at 22° C. in PBS.

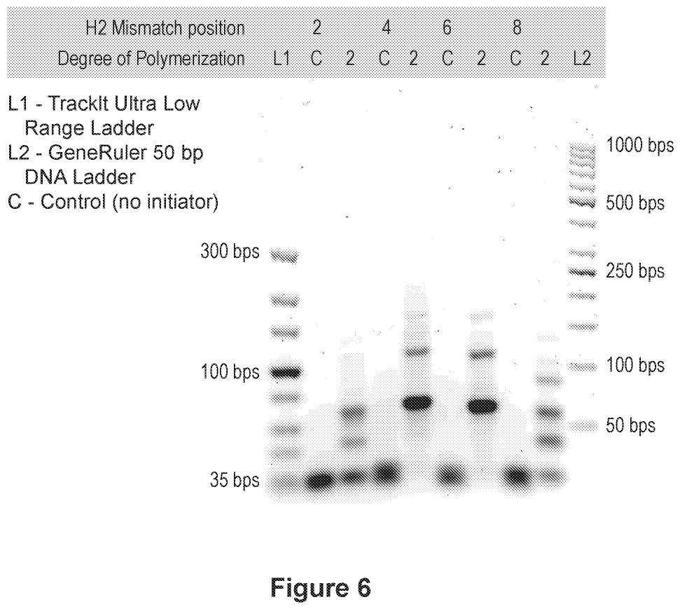

FIG. 7 shows agarose gel electrophoresis (3 w/w %) of reactions comparing monomer [either no mismatch or mismatch position 4 (H1m and H2m)] initiator (either I, I2, or I2B) and targeting a degree of polymerization of 2 or 4. Reactions conducted at 5 μM per sequence for 4 hours at 22° C. in PBS.

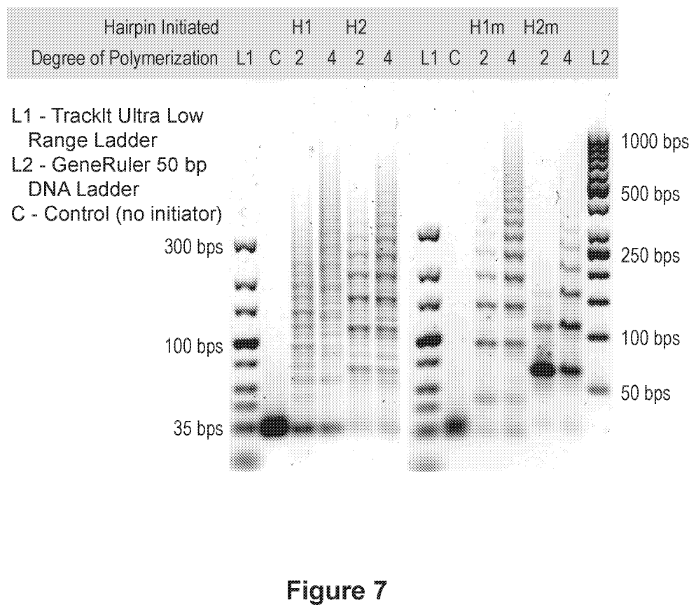

FIG. 8 shows size exclusion chromatography of reactions comparing monomer [either no mismatch or mismatch position 4 (H1m and H2m)] initiator (either I, I2, or I2B) and targeting a degree of polymerization of 2. Reactions conducted at 5 μM per sequence for 4 hours at 22° C. in PBS.

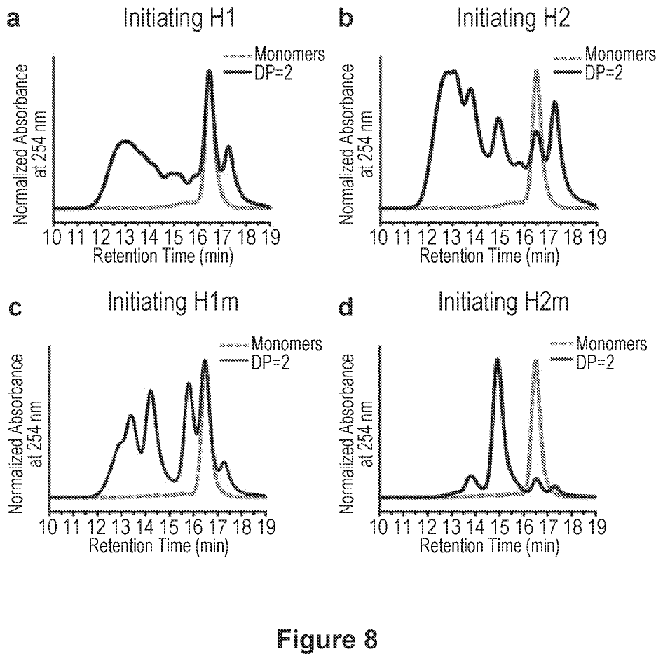

FIG. 9 shows size exclusion chromatography of I2b, H1m, and H2m showing increasing degree of polymerization, but not complete monomer conversion after 4 hours. Reactions conducted at 5 μM per sequence at 22° C. in PBS.

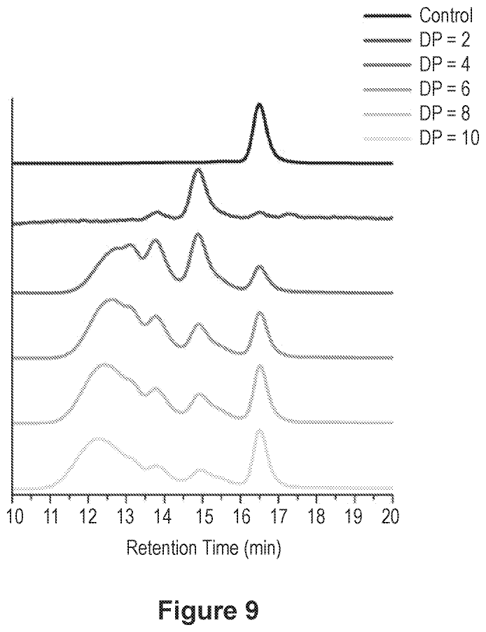

FIG. 10 shows agarose gel electrophoresis of I2b, H1m, and H2m showing increasing degree of polymerization and high conversions. Reactions conducted at 5 μM per sequence for 16 hours at 37° C. in PBS+0.5 M NaCl.

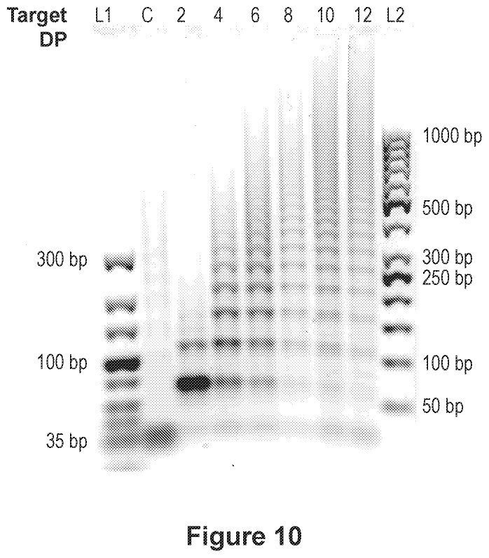

FIG. 11 shows size exclusion chromatography of I2b, H1m, and H2m showing efficient chain extensions from a first block with DP=2.9. Reactions conducted at 5 μM per sequence for 24 hours at 37° C. in PBS+1 M NaCl. Subsequently, monomer concentration was restored to 5 μM by adding fresh H1m and H2m and incubated for 24 hours at 37° C. in PBS+1 M NaCl. Finally, monomer was restored to 10 μM by adding fresh H1m and H2m and incubated for 24 hours at 37° C. in PBS+1 M NaCl.

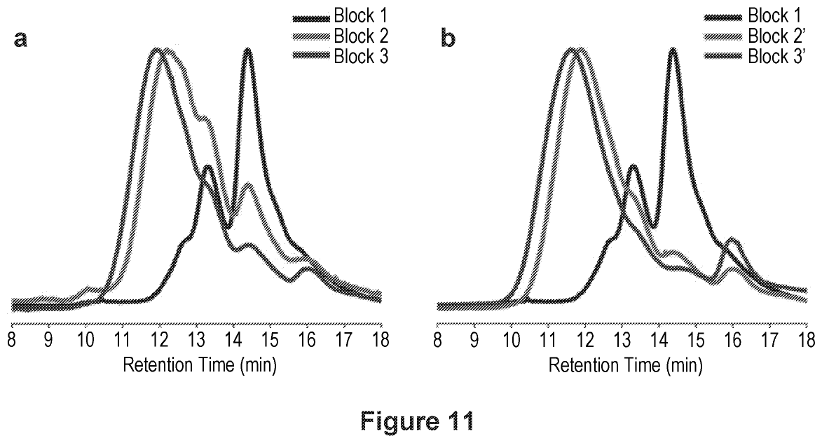

FIG. 12 shows agarose gel electrophoresis of I2b, H1m, and H2m showing efficient chain extensions from a first block with DP=6.9, but incomplete chain extensions when targeting DPs>18. Reactions conducted at 5 μM per sequence for 24 hours at 37° C. in PBS+1 M NaCl. Subsequently, monomer concentration was restored to 5 μM by adding fresh H1 m and H2m and incubated for 24 hours at 37° C. in PBS+1 M NaCl. Finally, monomer was restored to 10 μM by adding fresh H1 m and H2m and incubated for 24 hours at 37° C. in PBS+1 M NaCl.

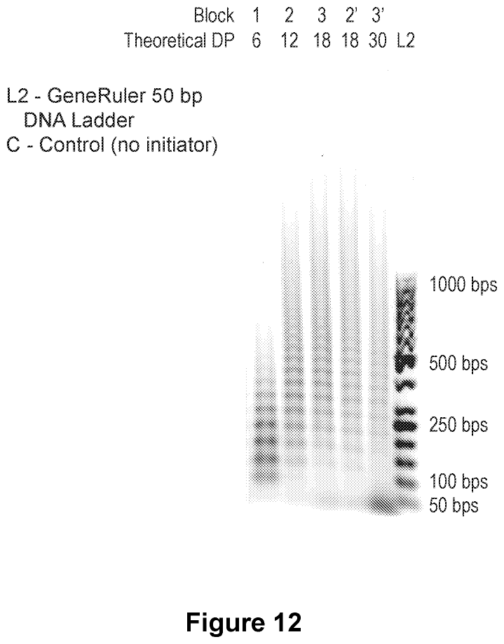

FIG. 13 shows oligomerization of a green fluorescent protein mutant conjugated with the new DNA monomers containing a mismatch compared to the old monomer system without a mismatch.

FIG. 14 depicts how the introduction of a mismatch into the duplexed stem region of DNA hairpins enables controlled DNA hybridization chain reaction (HCR) oligomerization.

FIG. 15 shows (a) DNA hairpin oligomerization pathway for hairpin 1 (H1) and hairpin 2 (H2) using an initiator (I1) complementary to H1; (b) DNA hairpin oligomerization mechanism of hairpin 1 with a mismatch (H1₄) and hairpin 2 with a mismatch (H2₄) using the complementary initiator to H1₄ (I1); (c) DNA hairpin oligomerization pathway for H1 and H2 using an initiator (I2) complementary to H2; (d) DNA hairpin oligomerization pathway for H1₄ and H2₄ using the complementary initiator to H2₄ (I2₄).

FIG. 16 shows example size exclusion chromatograms according to mismatch position using stoichiometric amounts of initiator, H1, and H2 to target a Degree of Polymerization=2. Entry numbers shown in FIG. 16 relate to Table 4.

FIG. 17 depicts the hybridization chain reaction mechanism showing that initiating and propagating species are chemically and/or energetically identical, leading to uncontrolled polymerizations.

FIG. 18 shows (a) a schematic of if initiation (k₁) is slow or relatively the same rate as propagation (k₂), oligomer product distribution would be more disperse because not all of monomer H₁ is reacted before H₂ is reacted. (b) Schematic of if k₁ is faster than k₂, all of monomer H₁ will be reacted before any H₂ is reacted, so low dispersity oligomerization products will be observed.

FIG. 19 shows (a) a schematic showing position and identity of mismatch incorporated into each set of hairpins; (b) Example agarose gel electrophoresis (3 w/w %) of reactions containing I1, H1, and H2 to initiate hairpin 1 and determine the effect of mismatch position on product distribution of reactions conducted at 5 μM per sequence for 4 h at 22° C. in PBS; (c) gel densiometry analysis from agarose gel showing degree of polymerization (DP) and dispersity (D) according to mismatch position.

FIG. 20 shows (a) a schematic showing position and identity of mismatch incorporated into each set of hairpins; (b) Example agarose gel electrophoresis (3 w/w %) of reactions containing I2, H1, and H2 to initiate hairpin 2 and determine the effect of mismatch position on product distribution of reactions conducted at 5 μM per sequence for 4 h at 22° C. in PBS; (c) gel densiometry analysis from agarose gel showing degree of polymerization (DP) and dispersity (D) according to mismatch position.

FIG. 21 shows example agarose gel electrophoresis (3 w/w %) of reactions comparing monomer [either no mismatch or mismatch position 4 (H1₄ and H2₄)] initiator (either I1, I2, or I2₄) and targeting a degree of polymerization of 2 or 4. Reactions conducted at 5 μM per sequence for 4 h at 22° C. in PBS.

FIG. 22 shows size exclusion chromatography of reactions comparing initiating monomers containing either no mismatch (H1 and H2) or a mismatch a position 4 (H1₄ and H2₄) using the corresponding initiator (I1, I2, or I2₄) and targeting a degree of polymerization of 2. Reactions conducted at 5 μM per sequence for 4 h at 22° C. in PBS.

FIG. 23 shows (a) CD spectra of melting experiments of H1 containing a mismatch at different positions obtained by heating from 25-90° C. in PBS; (b) Normalized m° at 245 nm of CD spectra to show melting transitions.

FIG. 24 shows (a) CD spectra of melting experiments of H2 containing a mismatch at different positions obtained by heating from 25-90° C. in PBS; (b) Normalized m° at 245 nm of CD spectra to show melting transitions.

FIG. 25 shows (a) CD spectra of melting experiments of H1₄ and H2₄ heating from 25-90° C. in PBS+0.5 M NaCl; (b) Normalized m° at 245 nm of CD spectra to show melting transitions emerge at lower temperatures that were attributed to the melting of the bases between the toe-hold and the mismatch.

FIG. 26 shows (a) CD spectra of melting experiments of H1₄ and H2₄ heating from 25-80° C. in PBS+1 M NaCl; (b) Normalized m° at 245 nm of CD spectra to show melting transitions emerge at lower temperatures that were attributed to the melting of the bases between the toe-hold and the mismatch.

FIG. 27 shows schemes of kinetic quenching experiments of Cy3 (Pink/purple star) hairpins using BHQ (black circle) quenching strands measuring the initiation/propagation of the conventional no mismatch system (a), the initiation of the mismatch system (b), or the propagation of the mismatch system (c). (d) Kinetics experiments monitoring the Cy3 fluorescence at 25° C. in PBS (20 nM Cy3-hairpin, 5 μM BHQ quenching strand).

FIG. 28 shows (a) Fluorescence quenching kinetics of strand displacement reaction of H1-Cy3 by I1-BHQ according to different concentrations; (b) fluorescence quenching kinetics of strand displacement reaction of H2₄-Cy3 by I2₄-BHQ according to different concentrations; (c) fluorescence quenching kinetics of strand displacement reaction of H2₄-Cy3 by P2₄-BHQ according to different concentrations; (d) plot of k_{obs} versus concentration calculated from fluorescence quenching kinetic studies showing that k_{obs} is independent of hairpin concentration.

FIG. 29 shows (a) Example agarose gel electrophoresis (3 w/w %) of I2₄, H1₄, and H2₄ showing increasing degree of polymerization and high conversions of reactions conducted at 5 μM per sequence for 16 h at 22° C. in PBS. (b) Gel densiometry analysis from agarose gel showing increasing DPₙ and DP_{w} according to expected degree of polymerization.

FIG. 30 shows (a) Example agarose gel electrophoresis (2 w/w %) of I2₄, H1₄, and H2₄ showing increasing degree of polymerization and high conversions of reactions conducted at 5 μM per sequence for 16 h at 37° C. in PBS+0.5 M NaCl. (b) Gel densiometry analysis from agarose gel showing increasing DPₙ and DP_{w} according to expected degree of polymerization.

FIG. 31 shows (a) Example agarose gel electrophoresis (1 w/w %) of I2₄, H1₄, and H2₄ showing increasing degree of polymerization and high conversions of reactions conducted at 5 μM per sequence for 16 h at 37° C. in PBS+1 M NaCl. (b) Gel densiometry analysis from agarose gel showing increasing DPₙ and DP_{w} according to expected degree of polymerization.

FIG. 32 shows results of experiments targeting different degrees of polymerization (DP) using I2₄, H1₄, and H2₄ in solutions with varying ionic strength. (a) Plot of resulting DPₙs compared to expected values and (b) dispersity according to varying [monomer]:[initiator]. (c) Chain extensions (Block 2 and Block 3) of a DNA oligomer (Block 1).

FIG. 33 shows oligomerization reactions targeting different degrees of polymerization using the conventional monomers without a mismatch (H1, H2, I1) for 16 h at 22° C. in PBS. (a) schematic of hairpin oligomerization without mismatches; (b) agarose gel electrophoresis (2 w/w %) showing a shift in band distribution according to targeted degree of polymerization; (c) gel densiometry analysis from agarose gel showing DPₙ and DP_{w} values of obtained DNA oligomers; (d) average experimental degree of polymerization and dispersity according to monomer to initiator ratio of 3 separate oligomerizations. Targeted DPs of 8 and 10 could not be resolved by analysis software to calculate degree of polymerization and dispersity. Odd values of DPₙ are grouped with the next highest even value in (c) for analysis (i.e., DPₙ s of 1 and 2 are grouped as DPₙ=2, DPₙ s of 3 and 4 are grouped as DPₙ=4, etc.).

FIG. 34 shows oligomerization reactions targeting different degrees of polymerization using monomers with a mismatch at position 2 (H1₂, H2₂, I2₂) for 16 h at 37° C. in PBS+0.5 M NaCl. (a) agarose gel electrophoresis (2 w/w %) showing a shift in band distribution according to targeted degree of polymerization; (b) gel densiometry analysis from agarose gel showing DPₙ and DP_{w} values of obtained DNA oligomers; (c) average experimental degree of polymerization and dispersity according to monomer to initiator ratio of 3 separate oligomerizations.

FIG. 35 shows oligomerization reactions targeting different degrees of polymerization using monomers with a mismatch at position 6 (H1₆, H2₆, I2₆) for 16 h at 37° C. in PBS+0.5 M PBS. (a) agarose gel electrophoresis (2 w/w %) showing a shift in band distribution according to targeted degree of polymerization; (b) gel densiometry analysis from agarose gel showing DPₙ and DP_{w} values of obtained DNA oligomers; (c) average experimental degree of polymerization and dispersity according to monomer to initiator ratio of 3 separate oligomerizations.

FIG. 36 shows oligomerization reactions targeting different degrees of polymerization using monomers with a mismatch at position 8 (H1₈, H2₈, I2₈) for 16 h at 37° C. in PBS+0.5 M NaCl. (a) agarose gel electrophoresis (2 w/w %) showing a shift in band distribution according to targeted degree of polymerization; (b) gel densiometry analysis from agarose gel showing DPₙ and DP_{w} values of obtained DNA oligomers; (c) average experimental degree of polymerization and dispersity according to monomer to initiator ratio of 3 separate oligomerizations.

FIG. 37 shows size exclusion chromatography of I2₄, H1₄, and H2₄ showing efficient chain extensions from a first block with DP=2.9. Reactions conducted at 5 μM per sequence for 24 h at 37° C. in PBS+1 M NaCl. Subsequently, monomer concentration was restored to 5 μM by adding fresh H1₄ and H2₄ and incubated for 24 h at 37° C. in PBS+1 M NaCl. Finally, monomer was restored to 10 μM by adding fresh H1₄ and H2₄ and incubated for 24 h at 37° C. in PBS+1 M NaCl.

FIG. 38 shows (a) Agarose gel electrophoresis (1 w/w %) of I2₄, H1₄, and H2₄ showing efficient chain extensions, but incomplete chain extensions when targeting DPs>18 (Block 3′″); (b, c) Normalized band intensity plots of two separate oligomer chain extensions. Reactions conducted at 5 μM per sequence for 24 h at 37° C. in PBS+1 M NaCl. ^{a}Gel resolution inhibited accurate DPₙ determination.

FIG. 39 shows (a) synthesis of mutant green fluorescent protein-DNA monomers (mGFP-H1₄, mGFP-H2₄); (b) scheme and (c) size-exclusion chromatogram of oligomerization and subsequent chain extension with mGFP-H1₄ and mGFP-H2₄; (d) size-exclusion chromatogram of oligomerization and subsequent chain extension with conventional mGFP-DNA monomers.

FIG. 40 shows a SDS-PAGE gel of mGFP-DNA H1 and H2 monomers. Lanes: L—Ladder, G—mGFP, H1—mGFP-H1 conjugate, H2—mGFP-H2 conjugate.

FIG. 41 depicts (a) synthesis of mutant green fluorescent protein-DNA monomers without mismatches (mGFP-H1, mGFP-H2); (b) scheme of oligomerization and subsequent chain extension of mGFP-H1 and mGFP-H2.

FIG. 42 shows an analysis of oligomerization using ImageJ software to plot gel band intensity.

FIG. 43 shows results in which area under each band peak was calculated according to arbitrary values assigned by ImageJ software, and each degree of polymerization was normalized to total area calculated.

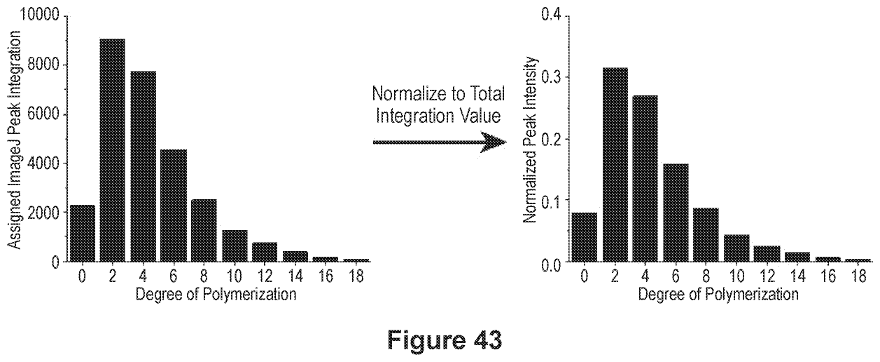

FIG. 44 shows a determination of the number-average degree of polymerization (DP, Equation 1), weight-average degree of polymerization (DP_{w}, Equation 2), and dispersity (D, Equation 3).

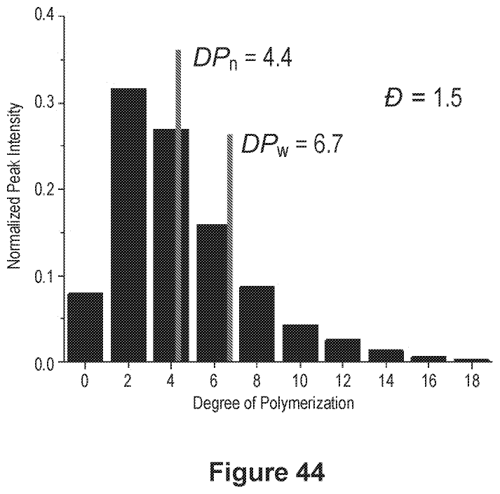

FIG. 45 shows SDS-PAGE gel of mGFP-DNA H1₄ and H2₄ monomers. Lanes: L—Ladder, G—mGFP, H1₄—mGFP—H1₄ conjugate, H2₄—mGFP—H2₄ conjugate.

FIG. 46 depicts size exclusion chromatography of I2₄, H1₄, and H2₄ showing increasing degree of polymerization. Reactions conducted at 5 μM per sequence at 22° C. in PBS.

FIG. 47 shows an abbreviated mechanism of the hybridization (hairpin) chain reaction (HCR) where an initiating DNA strand is used to open kinetically trapped DNA hairpins, beginning a chain reaction of hairpin openings to form DNA oligomers (See also FIGS. 15, 17, and 18) for more details on the HCR mechanism). Previous work [McMillan et al., JACS 2018, 140, 15950-15956] used this technique to oligomerize green fluorescent protein (GFP) by conjugating a single DNA hairpin to a surface cysteine of GFP (GFP-DNA conjugates). Upon introduction of an initiating DNA strand, oligomerization of GFP occurred where molecular weight (Mn) was dependent on initiator concentration.

FIG. 48 depicts a short oligomer of GFP that was synthesized using GFP-DNA monomers. Additional monomer of either GFP (See FIG. 39) or bovine serum albumin (BSA) was added to synthesize protein block copolymers. However, due to the poor control over oligomerization, size-exclusion chromatograms showed disperse products of both protein block copolymers. When oligomeric block copolymers of green fluorescent protein (GFP) were chain-extended with either another block of GFP or rhodamine-labelled bovine serum albumin (BSA), poor control over blocking efficiency was observed. Although the polymer chain ends were living, the polymerization only met one of the three general requirements for controlled living supramolecular polymerization (living chain ends, predictable molecular weights, well-defined block copolymers).

FIG. 49 shows a schematic of an ideal protein block copolymerization that would yield well defined oligomers with one protein in one portion of DNA and a second protein (either the same or different) in a second portion of DNA. If synthesis is inefficient (e.g., due to poor oligomer initiation) then a variety of byproducts will result. These byproducts would be a mixture of DNA and protein-DNA conjugates that are difficult to remove from the desired product.

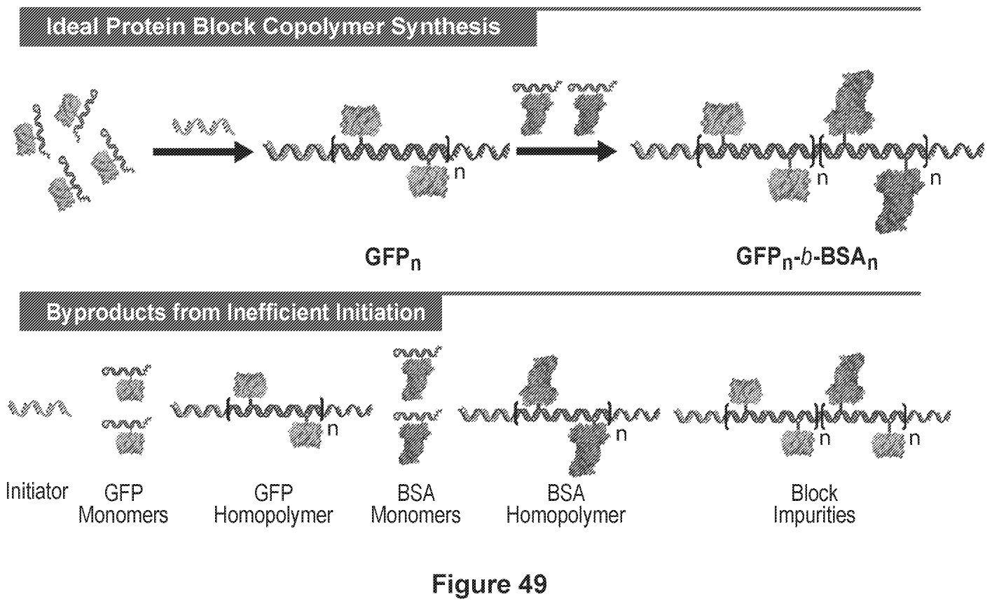

FIG. 50 is a schematic showing that by introducing a mismatch into conventional DNA hairpins, controlled oligomerization of DNA monomers can be realized when using a complementary initiating strand (FIG. 14). Initiating different hairpins (either Hairpin 1 (H1), Hairpin 2 (H2), mismatched Hairpin 1 (H1m or H14 herein), or mismatched hairpin 2 (H2m or H24 herein)) led to different size-exclusion chromatogram profiles. These profiles showed that initiating H2m yielded near molecularly pure dimers (see FIG. 16 and FIG. 22). Initiation was studied by analyzing the polymer distributions early in the polymerization of the DNA hairpins. Here, stoichiometric amounts of monomer to initiator were added to target a theoretical degree of polymerization (DP) of 1.

FIG. 51 shows that differences in initiation rate are proposed to result from hairpin design. Because H2m contains less G-C content, duplex fraying should occur more frequently because of the lower binding strength of A-T base pairs. Addition of the mismatch exacerbated this fraying, leading to an extended toehold length and shortened duplex length of the hairpins. This change in hairpin structure is contemplated to be a leading cause of increased initiation for H2m. For additional detail on hairpin design, see Table 6 and FIG. 19a. FIG. 27 provides evidence of increased rates of initiation resulting from the introduction of mismatch.

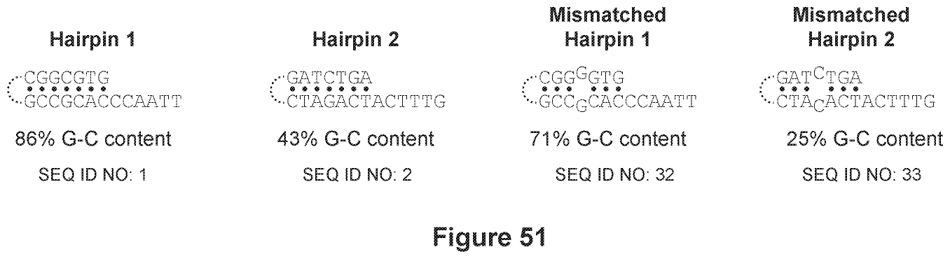

FIG. 52 shows results of experiments in which the stoichiometry of the mismatched hairpins was varied when initiating hairpin 2 to determine the monomer conversion and agreement between theoretical and experimental molecular weights according to target DP and molar mass dispersity (M_{w}/Mₙ). Changing monomer concentration when initiating H2m resulted in good agreement with theoretical degree of polymerization up to a degree of polymerization of 5. Low dispersity and high monomer conversion were also observed for these DNA oligomerizations. See FIGS. 29-36 for additional details. In this figure, DP is defined as the number of repeats of H1m and H2m together (e.g., DP 2 corresponds to DPₙ=4 and DP 3 corresponds to DPₙ=6 in FIG. 29).

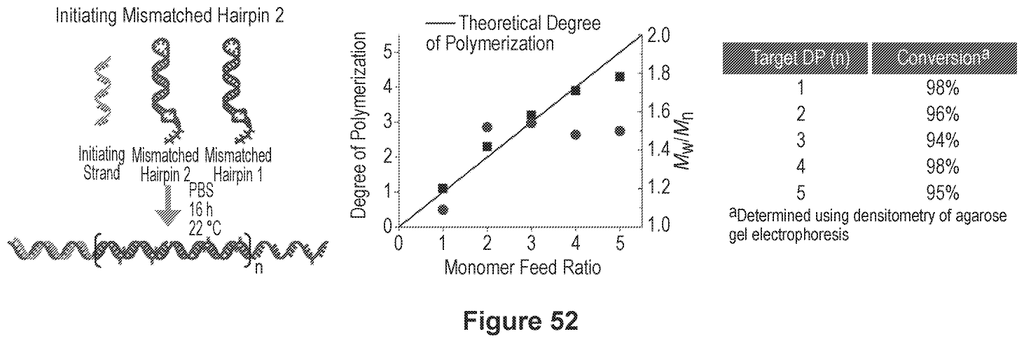

FIG. 53 shows that protein block copolymer synthesis of GFP-GFP was achieved with high purity using hairpin design methods disclosed herein that contain a mismatch (See also FIG. 39a-39c, and compare with FIG. 48 and FIG. 39d when mismatches were not present). Here, using mismatch hairpin monomers, polymerization of GFP was investigated by initiating hairpin 2. Polymerization was observed to proceed similar to DNA polymerization without protein, and good blocking efficiency with GFP was achieved.

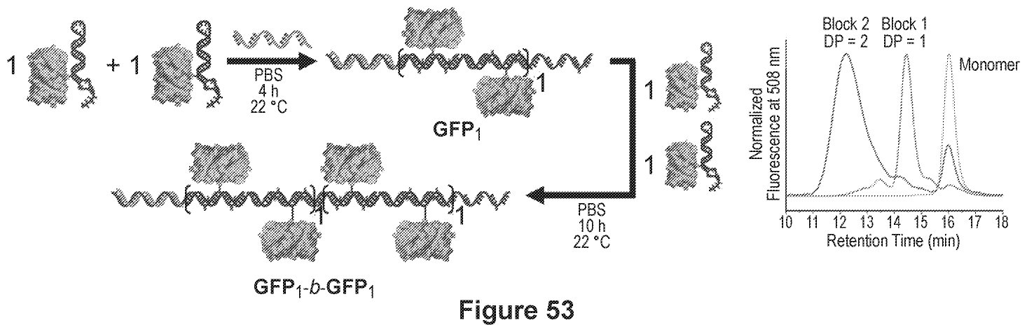

FIG. 54 shows results of experiments investigating the block efficiency using BSA monomers conjugated with the mismatched DNA hairpins, and improved block efficiency suggested well-defined GFP-b-BSA protein block copolymers. Thus, results showed that protein block copolymer synthesis of GFP-BSA was achieved with high purity using hairpin design methods disclosed herein that contain a mismatch (compare with FIG. 48 when mismatches were not present).

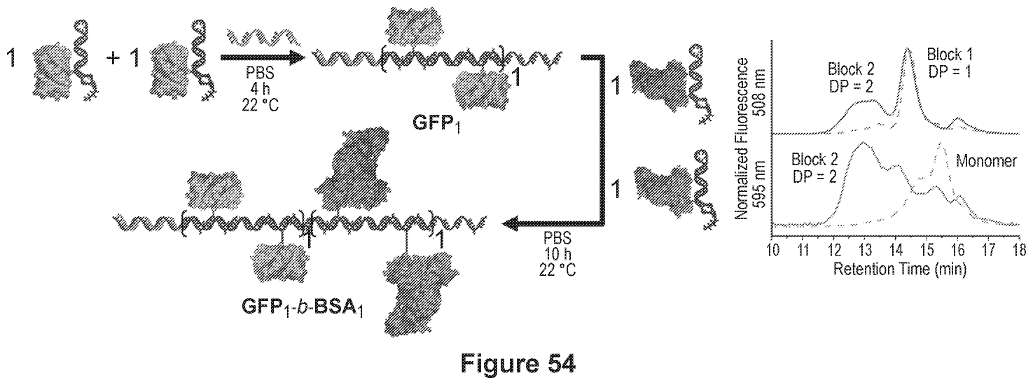

### DETAILED DESCRIPTION

Nature can organize proteins into precise oligomeric and polymeric supramolecular constructs containing multiple proteins with sequence specificity and defined degrees of polymerization. Analogous well-defined polymeric constructs are achievable on the molecular basis using controlled polymerization techniques. Indeed, (block)copolymers containing different functional monomers, polymer architecture, sequence, solvophilicity, molar mass, and dispersity are easily manipulated using common chain-growth polymerization techniques (e.g., anionic, cationic, metathesis, radical). Compared to small molecule monomer units, the complex sequences and functions of proteins introduces magnitudes more difficulty during polymerization, and well-defined molecular weight and topological control over synthetic protein polymerization was not previously realized. Recently, the DNA-mediated hairpin chain reaction (HCR) [Dirks et al., PNAS 2004, 101, 15275-15278] was employed to perform the chain-growth polymerization of proteins with programmable kinetically-trapped DNA hairpins [McMillan et al., JACS 2018, 140, 15950-15956].

As used in this specification and the appended claims, the singular forms “a,” “an,” and “the” include plural reference unless the context clearly dictates otherwise.

The terms “polynucleotide” and “oligonucleotide” are interchangeable as used herein.

The term “toehold” as used herein refers to a single-stranded nucleotide sequence that overhangs the duplex stem portion of an oligonucleotide hairpin monomer as described herein.

A “monomer” as used herein is a single oligonucleotide that typically comprises one or more portions that are substantially complementary to at least one other monomer being used in a hybridization chain reaction. Monomers of the disclosure are typically hairpin structures comprising a toehold, a duplex stem, and a hairpin loop. Typically, at least two monomers are used in HCR, but 3, 4, 5, 6, 7, 8, or more monomers may be used. Typically, a monomer is “metastable,” meaning that in the absence of an initiator oligonucleotide it is kinetically disfavored from associating with other monomers comprising substantially complementary regions. Monomers of the disclosure are those that are able to assemble into an oligomeric structure upon contact with an initiator oligonucleotide.

As used herein, an “initiator” is an oligonucleotide that initiates hybridization of monomers. Typically, an initiator oligonucleotide comprises a nucleic acid sequence that is substantially complementary to a portion of a monomer.

As used herein, “duplex” refers to a region in two complementary or substantially complementary oligonucleotides that form base pairs with one another, either by Watson-Crick base pairing or any other manner that allows for a stabilized duplex between oligonucleotide strands that are complementary or substantially complementary. “Substantially complementary” refers to the degree of complementarity between two nucleotide sequences such that a stable duplex is formed under the conditions in which the duplex is used. In various embodiments, substantially complementary nucleotide sequences are sequences that are or are at least 50%, 55%, 60%, 65%, 70%, 75%, 80%, 85%, 90%, 95%, or 99% complementary within a duplex. In some embodiments, substantially complementary nucleotide sequences are sequences that are 100% complementary within a duplex. In further embodiments, two nucleotide sequences are substantially complementary when there are 1, 2, 3, 4, 5, 6, 7, 8, 9, 10, 11, 12, 13, 14, 15 or more mismatches between the two nucleotide sequences within the duplex.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Oligonucleotide Hairpin Monomers

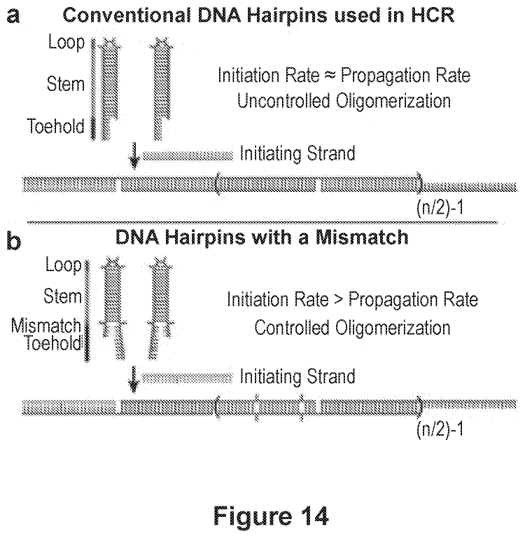

As disclosed herein, a monomer is a single oligonucleotide that typically comprises one or more portions that are substantially complementary to at least one other monomer being used in a hybridization chain reaction. Monomers are able to assemble into an oligomeric structure upon contact with an initiator oligonucleotide. In general, an oligonucleotide hairpin monomer of the disclosure comprises three portions—a toehold, a duplex stem, and a hairpin loop. See, e.g., FIG. 14. In some embodiments, the oligonucleotide hairpin monomer does not comprise a toehold. In various embodiments, an oligonucleotide hairpin monomer is from about 10 to about 200 nucleotides in length. In further embodiments, an oligonucleotide hairpin monomer is from about 10 to about 190, or about 10 to about 180, or about 10 to about 170, or about 10 to about 160, or about 10 to about 150, or about 10 to about 140, or about 10 to about 130, or about 10 to about 100, or about 10 to about 90, or about 10 to about 80, or about 10 to about 70, or about 10 to about 60, or about 10 to about 50, or about 10 to about 40, or about 10 to about 30, or about 10 to about 20, or about 20 to about 130, or about 20 to about 100, or about 20 to about 90, or about 20 to about 80, or about 20 to about 70, or about 20 to about 60, or about 20 to about 50, or about 20 to about 40, or about 20 to about 30 nucleotides in length. In further embodiments, an oligonucleotide hairpin monomer is, is about, or is at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, 110, 120, 130, 140, 150, 160, 170, 180, 190, 200, or more nucleotides in length. In further embodiments, an oligonucleotide hairpin monomer is less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, 110, 120, 130, 140, 150, 160, 170, 180, 190, or 200 nucleotides in length. In some embodiments, the oligonucleotide hairpin monomer comprises a base-pair mismatch in the duplex stem. In various embodiments, the base-pair mismatch is located at a position that is proximal to the toehold and distal to the hairpin loop, relative to a midpoint of the first duplex stem. In some embodiments, the base-pair mismatch in a duplex stem is located at a position that is 1, 2, 3, 4, 5, 6, 7, 8, 9, 10, 11, 12, 13, 14, 15, 16, 17, 18, 19, 20, 21, 22, 23 24, 25, or more nucleotides from the toehold. In some embodiments, the base-pair mismatch in a duplex stem is located at a position that is 4 nucleotides from the toehold. In various embodiments, the duplex stem comprises 2, 3, 4, or 5 base-pair mismatches. In some embodiments, the duplex stem comprises 2 base-pair mismatches that are 3 nucleotides apart from each other. In some embodiments, the duplex stem comprises 3, 4, or 5 base-pair mismatches and each mismatch is 3-4 nucleotides apart from another base-pair mismatch.

In various embodiments, the toehold comprises or consists of about 1 to about 50 nucleotides. In some embodiments, the toehold is about 4 to about 12 nucleotides in length. In further embodiments, the toehold is or is about 1, 2, 3, 4, 5, 6, 7, 8, 9, 10, 11, 12, 13, 14, 15, or more nucleotides in length. In further embodiments, the toehold is at least about 1, 2, 3, 4, 5, 6, 7, 8, 9, 10, 11, 12, 13, 14, or 15 nucleotides in length. In further embodiments, the toehold is less than about 2, 3, 4, 5, 6, 7, 8, 9, 10, 11, 12, 13, 14, or 15 nucleotides in length.

The duplex stem, in various embodiments, comprises or consists of about 10 to about 50 nucleotides. In some embodiments, the duplex stem is about 18 to about 24 nucleotides in length. In some embodiments, the duplex stem is or is about 18 nucleotides in length. In further embodiments, the duplex stem is or is about 10, 15, 20, 25, 30, 35, 40, 45, 50, 75, 100, or more nucleotides in length. In further embodiments, the duplex stem is at least about 10, 15, 20, 25, 30, 35, 40, 45, 50, 75, 100, or more nucleotides in length. In further embodiments, the duplex stem is less than about 10, 15, 20, 25, 30, 35, 40, 45, 50, 75, or 100 nucleotides in length.

The hairpin loop, in various embodiments, comprises or consists of about 3 to about 30 nucleotides. In some embodiments, the hairpin loop is about 4 to about 12 nucleotides in length. In further embodiments, the hairpin loop is about 3, 4, 5, 6, 7, 8, 9, 10, 11, 12, 13, 14, 15, 16, 17, 18, 19, 20, 25, 30, or more nucleotides in length. In some embodiments, the hairpin loop is at least 3, 4, 5, 6, 7, 8, 9, 10, 11, or 12 nucleotides in length. In further embodiments, the hairpin loop is less than 4, 5, 6, 7, 8, 9, 10, 11, or 12 nucleotides in length.

Thus, in some aspects, the disclosure provides a first oligonucleotide hairpin monomer and a second oligonucleotide hairpin monomer, the first oligonucleotide hairpin monomer comprising a first toehold, a first duplex stem, and a first hairpin loop, wherein the first duplex stem comprises a base-pair mismatch at a position that is proximal to the first toehold and distal to the first hairpin loop, relative to a midpoint of the first duplex stem, and the second oligonucleotide hairpin monomer comprising a second toehold, a second duplex stem, and a second hairpin loop. In some embodiments, the second oligonucleotide hairpin monomer comprises a base-pair mismatch. In some embodiments, the second hairpin loop and the first toehold are about 70%, about 80%, about 90%, about 99%, or 100% complementary to each other. In further embodiments, the second toehold and the first hairpin loop are about 70%, about 80%, about 90%, about 99%, or 100% complementary to each other.

In some aspects, the disclosure provides a first oligonucleotide hairpin monomer and a second oligonucleotide hairpin monomer, the first oligonucleotide hairpin monomer comprising a first duplex stem comprising nucleotide sequence B and nucleotide sequence C that are substantially complementary to each other, a first hairpin loop at a first end of the first duplex stem, and a toehold comprising nucleotide sequence A at a second end of the first duplex stem, wherein the first duplex stem comprises a base-pair mismatch at a position that is proximal to nucleotide sequence A and distal to the first hairpin loop, relative to a midpoint of the first duplex stem, and the second oligonucleotide hairpin monomer comprising a second duplex stem comprising nucleotide sequence G and nucleotide sequence H that are substantially complementary to each other, a second hairpin loop at a first end of the second duplex stem that is substantially complementary to nucleotide sequence A of the first oligonucleotide hairpin monomer. In some embodiments, the second oligonucleotide hairpin monomer comprises a base-pair mismatch.

### Initiator Oligonucleotides

As described herein, an initiator is an oligonucleotide that initiates hybridization of monomers. Typically, an initiator oligonucleotide comprises a nucleic acid sequence that is substantially complementary to a portion of a monomer. In various embodiments, the initiator oligonucleotide is about 10 to about 100 nucleotides in length. In further embodiments, the initiator nucleotide is, is about, or is at least about 10, 15, 20, 25, 30, 35, 40, 45, 50, 55, 60, 65, 70, 75, 80, 85, 90, 95, 100, or more nucleotides in length. In some embodiments, the initiator nucleotide is less than about 15, 20, 25, 30, 35, 40, 45, 50, 55, 60, 65, 70, 75, 80, 85, 90, 95, or 100 nucleotides in length.

In some aspects, the disclosure provides an initiator oligonucleotide comprising a first nucleotide sequence that is substantially complementary to the toehold of an oligonucleotide hairpin monomer, and a second nucleotide sequence that is substantially complementary to a portion of the duplex stem of the oligonucleotide hairpin monomer. The first nucleotide sequence that is substantially complementary to the toehold of an oligonucleotide hairpin monomer is, in various embodiments, about 1 to about 20 nucleotides in length. In some embodiments, the first nucleotide sequence that is substantially complementary to the toehold of an oligonucleotide hairpin monomer is about 4 to about 12 nucleotides in length. In further embodiments, the toehold of an oligonucleotide hairpin monomer is, is about, or is at least about 1, 2, 3, 4, 5, 6, 7, 8, 9, 10, 11, 12, 13, 14, 15, 16, 17, 18, 19, 20, or more nucleotides in length. In further embodiments, the toehold of an oligonucleotide hairpin monomer is less than about 2, 3, 4, 5, 6, 7, 8, 9, 10, 11, 12, 13, 14, 15, 16, 17, 18, 19, or 20 nucleotides in length. The second nucleotide sequence that is substantially complementary to a portion of the duplex stem of the oligonucleotide hairpin monomer is, in some embodiments, about 10 to about 50 nucleotides in length. In some embodiments, the second nucleotide sequence that is substantially complementary to a portion of the duplex stem of the oligonucleotide hairpin monomer is about 18 to about 24 nucleotides in length. In some embodiments, the second nucleotide sequence that is substantially complementary to a portion of the duplex stem of the oligonucleotide hairpin monomer is or is about 18 nucleotides in length. In further embodiments, the second nucleotide sequence that is substantially complementary to a portion of the duplex stem of the oligonucleotide hairpin monomer is or is about 10, 15, 20, 25, 30, 35, 40, 45, 50, 55, 60, 65, 70, 75, 80, or more nucleotides in length. In further embodiments, the second nucleotide sequence that is substantially complementary to a portion of the duplex stem of the oligonucleotide hairpin monomer is at least about 10, 15, 20, 25, 30, 35, 40, 45, 50, 55, 60, 65, 70, 75, 80, or more nucleotides in length. In further embodiments, the second nucleotide sequence that is substantially complementary to a portion of the duplex stem of the oligonucleotide hairpin monomer is less than about 15, 20, 25, 30, 35, 40, 45, 50, 55, 60, 65, 70, 75, or 80 nucleotides in length.

In some embodiments, the initiator oligonucleotide is an analyte to be detected in a sample. In further embodiments, the initiator oligonucleotide is single stranded. In some embodiments, the initiator oligonucleotide is RNA. In still further embodiments, the initiator oligonucleotide is messenger RNA (mRNA).

### Controlling the Hybridization Chain Reaction (HCR)

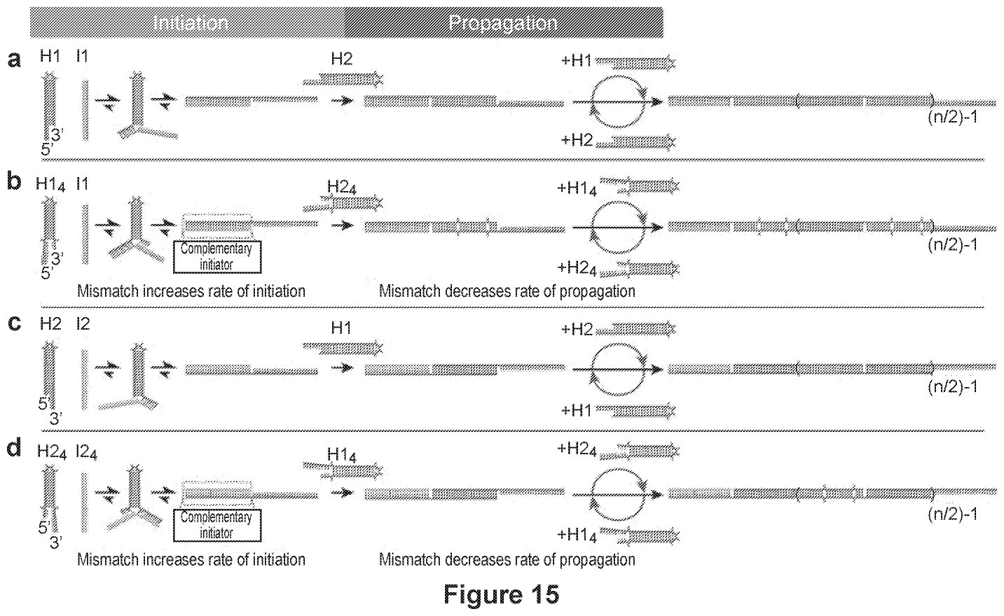

The disclosure provides materials and methods for controlling the hybridization chain reaction (HCR). HCR [Dirks et al., Proc. Natl. Acad. Sci. U.S.A 2004, 101, 15275-15278] mimics molecular chain-growth polymerization and only requires three distinct DNA sequences (the initiating sequence and 2 alternating monomer sequences, FIGS. 14a and 15a. In HCR, the secondary structure of DNA is utilized to increase the energy barrier associated with oligomerization. For example, two oligonucleotide hairpins containing complementary regions are kinetically trapped until the introduction of an initiator strand triggers a chain reaction of oligonucleotide duplexing via sequential strand displacement events. Prior to the present disclosure there has not been a way to predetermine the number of hairpin monomers that are incorporated into each oligomer (i.e., number-average degree of polymerization, DP) using HCR.

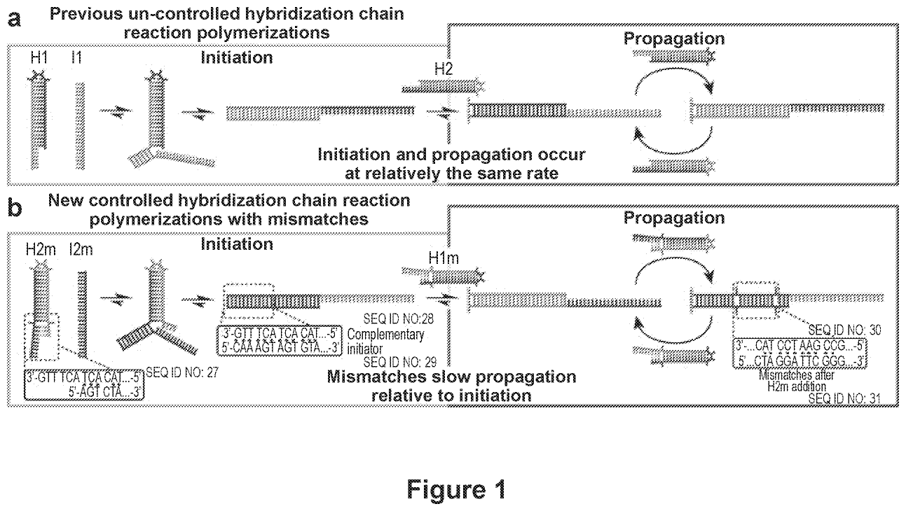

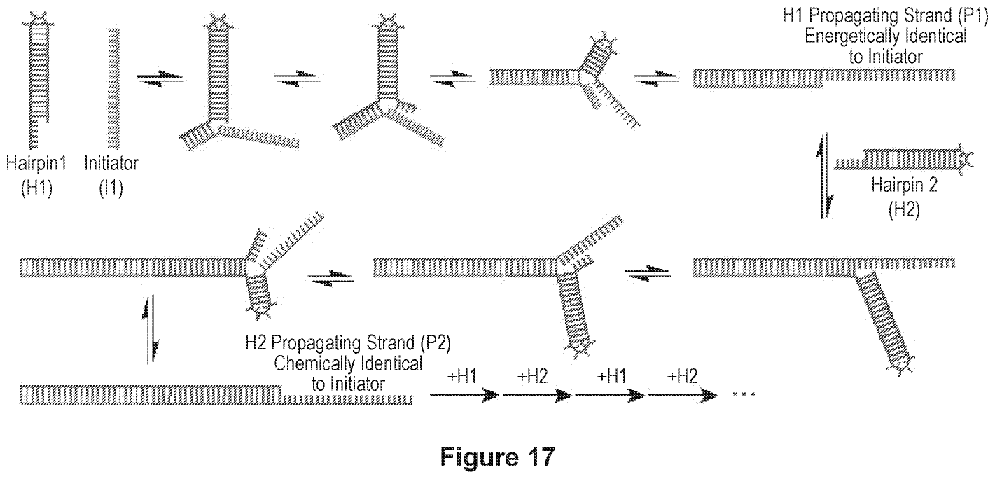

HCR polymerization inherently results in a living single-stranded DNA chain end, but the polymerizations usually deviate from most small-molecule controlled polymerization requirements (e.g., predictable molecular weights, low molar-mass dispersities, well-defined block copolymers). In typical HCR designs, the symmetry of the hairpins means that both initiating and propagating invader strands undergo the same number of branch migration steps to form duplexes identical in length (FIGS. 1A and 17). Thus, uncontrolled polymerizations result from energetically similar initiation and propagation steps.

DNA oligomerization using the hybridization chain reaction is extensively used in biosensing (e.g., mRNA expression levels), to catalyze molecular machines, and to organize nanoscale materials (e.g., proteins, quantum dots). Among the most impactful applications of HCR are in sensing of DNA or RNA. However, the oligomerization outcome is empirical due to design symmetry requirements of previous systems. This inhibits predictable degrees of oligomerization quantitatively related to reagent ratios. In other words, quantification of mRNA expression levels, relations between molecular machine activity and monomer fuel, and the number of nanoscale materials per DNA polymer are all experimentally determined and cannot be predicted. The present disclosure introduces control over DNA oligomerization, leading to predictable numbers of DNA monomers per oligomer. Control is achieved by slight modifications to DNA monomer sequences to delineate oligomerization steps. Specifically, the kinetics of initiation are differentiated from propagation. Resultantly, DNA oligomers with low dispersities can be predicted according to reagent ratios. Therefore, the present disclosure introduces quantitative relationships between reagent stoichiometry and DNA oligomerization outcome. Previous techniques do not allow predictable relations between input (e.g., mRNA) and output (e.g., fluorescence signal). However, since the polymerization products taught herein are directly related to reagent ratios, semi-quantitative measurements can be achieved.

Herein, it is disclosed that HCR polymerization can be controlled via the introduction of a single base-pair mismatch into the duplex (FIGS. 1b and 14). Two significant energetic consequences of the mismatch lead to control. First, the mismatch increases the length of the toehold due to increased base-pair fraying adjacent to the mismatch,^{13, 14} leading to fast initiation with a complementary sequence (FIG. 15).^{15, 16} Second, the mismatch of each hairpin can be translated into subsequent propagation events, slowing strand displacement.¹⁷⁻²⁰ Both effects should differentiate initiation and propagation energies, leading to a more controlled HCR polymerization.

In some aspects, methods of the disclosure comprise contacting an oligonucleotide hairpin monomer and an initiator oligonucleotide (“initiation”) which triggers sequential strand displacement events and interactions between the two oligonucleotide hairpin monomers and the initiator oligonucleotide (“propagation”). In some embodiments, the disclosure provides two or more oligonucleotide hairpin monomers and an initiator oligonucleotide that oligomerize in a controlled fashion. In general, oligonucleotide hairpin monomers and initiator oligonucleotides are designed such that they will hybridize in a chain reaction. For example, in a reaction comprising two oligonucleotide hairpin monomers and an initiator oligonucleotide, a first oligonucleotide hairpin monomer comprises (i) a first duplex stem comprising nucleotide sequence B and nucleotide sequence C that are substantially complementary to each other, (ii) a first hairpin loop at a first end of the first duplex stem, and (iii) a toehold comprising nucleotide sequence A at a second end of the first duplex stem, wherein the first duplex stem comprises a base-pair mismatch at a position that is proximal to nucleotide sequence A and distal to the first hairpin loop, relative to a midpoint of the first duplex stem; an initiator oligonucleotide comprises: (i) nucleotide sequence D that is substantially complementary to nucleotide sequence A of the first oligonucleotide hairpin monomer, and (ii) nucleotide sequence E that is substantially complementary to nucleotide sequence B of the first oligonucleotide hairpin monomer; and a second oligonucleotide hairpin monomer comprises: (i) a second duplex stem comprising nucleotide sequence G and nucleotide sequence H that are substantially complementary to each other, (ii) a second hairpin loop at a first end of the second duplex stem that is substantially complementary to nucleotide sequence A of the first oligonucleotide hairpin monomer, and (iii) a toehold comprising nucleotide sequence F at a second end of the second duplex stem that is substantially complementary to the first hairpin loop of the first oligonucleotide hairpin monomer. In a general description of the reaction, nucleotide sequence D on the initiating strand will interact with sequence A of the first hairpin monomer. Next, nucleotide sequence E will replace each base pair of the sequence B-C duplex to form a duplex comprising nucleotide sequences E and B, leading to displacement of strand C by strand E. The now single-stranded region comprising nucleotide sequence C and the first hairpin loop can interact with the second hairpin monomer. The loop of the first hairpin can interaction with the single-stranded nucleotide sequence F of the second hairpin to form a duplex. Next, oligonucleotide sequence C will replace each base pair of the sequence G-H duplex to form a duplex comprised of nucleotide sequences C and G, leading to displacement of strand H by strand C. If a mismatch is encountered during strand displacement, the next complementary base-pair forms. The now single-stranded region comprising nucleotide sequence H and the second hairpin loop can interact with the first hairpin monomer in a manner analogous to the initiating sequence. If a mismatch is encountered during strand displacement, the next complementary base-pair forms. This leads to alternating openings of the first and the second hairpins leading to duplexes with substantial complementarity.

Control is achieved, in any of the aspects or embodiments of the disclosure, through the introduction of a base-pair mismatch in at least one of the oligonucleotide hairpin monomers. In some embodiments, each oligonucleotide hairpin monomer comprises a base-pair mismatch. The introduction of the base-pair mismatch allows one to kinetically differentiate initiation versus propagation events, leading to structures of defined sizes (e.g., up to 10 monomers in length). Thus, one can control the oligomerization of monomers according to the amount of initiator and monomer added to the reaction. Methods of the disclosure also enable the production of populations of oligomers having low dispersity.

By way of non-limiting examples, to realize a controlled DNA oligomer with a predictable number of monomers per oligomer (e.g., 2, 4, or 6 monomer units), equal amounts of the first and second hairpin are added to a reaction. Subsequently, in the case of realizing an oligomer with 2 monomer units, an equal amount of initiator is added to the reaction. In the case of realizing an oligomer with 4 monomer units, one half the amount of initiator is added to the reaction. In the case of realizing an oligomer with 6 monomer units, one third the amount of initiator is added to the reaction. In general, for applications where signal is turned on via hairpin opening and high signal is desired (e.g., mRNA detection), the ratio of monomer to initiator should be high such that longer oligomers are achieved. In general, for applications where small oligomers are desired (e.g., oligomers of antibodies), the ratio of the monomer to initiator should be low to yield only a few monomers per oligomer.

Accordingly, in some aspects the disclosure provides a method of producing a structure, the method comprising contacting: a) a first oligonucleotide hairpin monomer comprising a first toehold, a first duplex stem, and a first hairpin loop, wherein the first duplex stem comprises a base-pair mismatch at a position that is proximal to the first toehold and distal to the first hairpin loop, relative to a midpoint of the first duplex stem; b) a second oligonucleotide hairpin monomer comprising a second toehold, a second duplex stem, and a second hairpin loop; and c) an initiator oligonucleotide, wherein hybridization of the initiator oligonucleotide to the first oligonucleotide hairpin monomer results in hybridization of the first oligonucleotide hairpin monomer to the second oligonucleotide hairpin monomer, thereby producing the structure. In some embodiments, the first oligonucleotide hairpin monomer and the second oligonucleotide hairpin monomer are in a solution and are then contacted with the initiator oligonucleotide in the solution. In some embodiments, the initiator oligonucleotide is contacted with the first oligonucleotide hairpin monomer in a solution and then the second oligonucleotide hairpin monomer is added to the solution. In some embodiments, the base-pair mismatch in the first duplex stem is located at a position that is 1, 2, 3, 4, 5, 6, 7, 8, 9, 10, 11, 12, 13, 14, or 15 nucleotides from the first toehold. In further embodiments, the first duplex stem comprises 2, 3, 4, or 5 base-pair mismatches. In some embodiments, the second duplex stem comprises a base-pair mismatch at a position that is proximal to the second toehold and distal to the second hairpin loop, relative to a midpoint of the second duplex stem. In further embodiments, the base-pair mismatch in the second duplex stem is located at a position that is 1, 2, 3, 4, 5, 6, 7, 8, 9, 10, 11, 12, 13, 14, or 15 nucleotides from the second toehold. In still further embodiments, the second duplex stem comprises 2, 3, 4, or 5 base-pair mismatches. In some embodiments, the first hairpin oligonucleotide monomer further comprises an additional moiety. In further embodiments, a second oligonucleotide hairpin monomer further comprises an additional moiety. In further embodiments, the initiator oligonucleotide further comprises an additional moiety. In some embodiments, the first oligonucleotide hairpin monomer further comprises an antibody and the second oligonucleotide hairpin monomer further comprises an additional antibody. In further embodiments, the antibody and the additional antibody are different. In some embodiments, the first oligonucleotide hairpin monomer is DNA, RNA, or a modified form thereof. In further embodiments, the second oligonucleotide hairpin monomer is DNA, RNA, or a modified form thereof. In still further embodiments, the initiator oligonucleotide is DNA, RNA, or a modified form thereof. In some embodiments, the method further comprises contacting the structure with a termination oligonucleotide. A “termination oligonucleotide” is an oligonucleotide that reacts with the oligomerized structure to prevent any subsequent monomer additions. The prevention of subsequent addition may be reversible or irreversible. In some embodiments, the termination oligonucleotide is a short sequence comprising a nucleotide sequence complementary to the loop and stem region that is not attached to the toehold (e.g., nucleotide sequence C or H as described herein). In further embodiments, the termination oligonucleotide comprises a nucleotide sequence that is complementary to the loop and stem region not attached to the toehold (e.g., nucleotide sequence C or H as described herein) and a sequence not complementary to any strands in the reaction. In various embodiments, the number of oligonucleotide hairpin monomers incorporated into the structure is or is about 2, 3, 4, 5, 6, 7, 8, 9, 10, 11, 12, 13, 14, 15, 20, 30, 40, 50, 60, 70, 80, 90, 100, or more monomers. In some embodiments, the number of oligonucleotide hairpin monomers incorporated into the structure is at least about 2, 3, 4, 5, 6, 7, 8, 9, 10, 11, 12, 13, 14, 15, 20, 30, 40, 50, 60, 70, 80, 90, 100, or more monomers. In further embodiments, the number of oligonucleotide hairpin monomers incorporated into the structure is less than about 3, 4, 5, 6, 7, 8, 9, 10, 11, 12, 13, 14, 15, 20, 30, 40, 50, 60, 70, 80, 90, or 100 monomers. In various embodiments, the number of oligonucleotide hairpin monomers incorporated into the structure is about 2 to 100, 2 to 50, 10 to 50, 2 to 50, 5 to 30, 2 to 15, 2 to 14, 2 to 10, 3 to 15, 3 to 14, 3 to 10, 4 to 15, 4 to 14, 4 to 10, 5 to 15, 5 to 14, 5 to 10, 6 to 15, 6 to 14, 6 to 10, 7 to 15, 7 to 14, 7 to 10, 3 to 6, 3 to 7, 3 to 8, 3 to 9, 4 to 6, 4 to 7, 4 to 8, or 4 to 9 monomers. In various embodiments, the dispersity of a plurality of structures is from about 1 to about 2.

In some aspects, the disclosure provides a method of producing a structure comprising hybridized oligonucleotide monomers, the method comprising contacting: a) a first oligonucleotide hairpin monomer comprising: (i) a first duplex stem comprising nucleotide sequence B and nucleotide sequence C that are substantially complementary to each other, (ii) a first hairpin loop at a first end of the first duplex stem, and (iii) a toehold comprising nucleotide sequence A at a second end of the first duplex stem, wherein the first duplex stem comprises a base-pair mismatch at a position that is proximal to nucleotide sequence A and distal to the first hairpin loop, relative to a midpoint of the first duplex stem; b) an initiator oligonucleotide comprising: (i) nucleotide sequence D that is substantially complementary to nucleotide sequence A of the first oligonucleotide hairpin monomer, and (ii) nucleotide sequence E that is substantially complementary to nucleotide sequence B of the first oligonucleotide hairpin monomer; and c) a second oligonucleotide hairpin monomer comprising: (i) a second duplex stem comprising nucleotide sequence G and nucleotide sequence H that are substantially complementary to each other, (ii) a second hairpin loop at a first end of the second duplex stem that is substantially complementary to nucleotide sequence A of the first oligonucleotide hairpin monomer, and (iii) a toehold comprising nucleotide sequence F at a second end of the second duplex stem that is substantially complementary to the first hairpin loop of the first oligonucleotide hairpin monomer, wherein hybridization of the initiator oligonucleotide to the first oligonucleotide hairpin monomer results in hybridization of the first oligonucleotide hairpin monomer to the second oligonucleotide hairpin monomer, thereby producing the structure. In some embodiments, the base-pair mismatch in the first duplex stem is located at a position that is 1, 2, 3, 4, 5, 6, 7, 8, 9, 10, 11, 12, 13, 14, or 15 nucleotides from the second end of the first duplex stem. In further embodiments, the first duplex stem comprises 2, 3, 4, or 5 base-pair mismatches. In some embodiments, the second duplex stem comprises a base-pair mismatch at a position that is proximal to nucleotide sequence F and distal to the second hairpin loop, relative to a midpoint of the second duplex stem. In further embodiments, the base-pair mismatch in the second duplex stem is located at a position that is 1, 2, 3, 4, 5, 6, 7, 8, 9, 10, 11, 12, 13, 14, or 15 nucleotides from the second end of the second duplex stem. In further embodiments, the second duplex stem comprises 2, 3, 4, or 5 base-pair mismatches. In some embodiments, nucleotide sequence D comprises or consists of about 1 to about 20 nucleotides. In further embodiments, nucleotide sequence D is or is about 4 to about 12 nucleotides in length. In some embodiments, nucleotide sequence D is, is about, or is at least about 1, 2, 3, 4, 5, 6, 7, 8, 9, 10 11, 12, 13, 14, 15, 16, 17, 18, 19, or 20 nucleotides in length. In some embodiments, nucleotide sequence D is less than about 2, 3, 4, 5, 6, 7, 8, 9, 10 11, 12, 13, 14, 15, 16, 17, 18, 19, or 20 nucleotides in length. In some embodiments, nucleotide sequence E comprises or consists of about 10 to about 50 nucleotides. In further embodiments, nucleotide sequence E is or is about 15, 16, 17, 18, 19, 20, 21, 22, 23, 24, or 25 nucleotides in length. In some embodiments, nucleotide sequence E is, is about, or is at least about 10, 15, 20, 25, 30, 35, 40, 45, 50, or more nucleotides in length. In some embodiments, nucleotide sequence E is less than about 10, 15, 20, 25, 30, 35, 40, 45, or 50. In some embodiments, nucleotide sequence B and nucleotide sequence C are about 70%, about 80%, about 90%, or about 99% complementary to each other. In any of the aspects or embodiments of the disclosure, nucleotide sequence B and nucleotide sequence C are not 100% complementary to each other. In further embodiments, nucleotide sequence D and nucleotide sequence A are about 70%, about 80%, about 90%, about 99%, or 100% complementary to each other. In some embodiments, nucleotide sequence E and nucleotide sequence B are about 70%, about 80%, about 90%, about 99%, or 100% complementary to each other. In some embodiments, nucleotide sequence G and nucleotide sequence H are about 70%, about 80%, about 90%, about 99%, or 100% complementary to each other. In some embodiments, the second hairpin loop and nucleotide sequence A are about 70%, about 80%, about 90%, about 99%, or 100% complementary to each other. In further embodiments, nucleotide sequence F and the first hairpin loop are about 70%, about 80%, about 90%, about 99%, or 100% complementary to each other. In some embodiments, the first hairpin oligonucleotide monomer further comprises an additional moiety. In further embodiments, a second oligonucleotide hairpin monomer further comprises an additional moiety. In further embodiments, the initiator oligonucleotide further comprises an additional moiety. In some embodiments, the first hairpin oligonucleotide monomer further comprises an antibody and the second oligonucleotide hairpin monomer further comprises an additional antibody. In further embodiments, the antibody and the additional antibody are different. In some embodiments, the structure is detected by gel electrophoresis, mass spectrometry, light scattering spectroscopy, colorimetry, fluorescent microscopy, fluorescent spectroscopy, electron microscopy, atomic force microscopy, nuclear magnetic resonance (NMR) depending or a combination thereof. In some embodiments, the first oligonucleotide hairpin monomer is DNA, RNA, or a modified form thereof. In further embodiments, the second oligonucleotide hairpin monomer is DNA, RNA, or a modified form thereof. In still further embodiments, the initiator oligonucleotide is DNA, RNA, or a modified form thereof. In further embodiments, the method further comprises contacting the structure with a termination oligonucleotide. In various embodiments, the number of oligonucleotide hairpin monomers incorporated into the structure is or is about 2, 3, 4, 5, 6, 7, 8, 9, 10, 11, 12, 13, 14, 15, 20, 30, 40, 50, 60, 70, 80, 90, 100, or more monomers. In some embodiments, the number of oligonucleotide hairpin monomers incorporated into the structure is at least about 2, 3, 4, 5, 6, 7, 8, 9, 10, 11, 12, 13, 14, 15, 20, 30, 40, 50, 60, 70, 80, 90, 100, or more monomers. In further embodiments, the number of oligonucleotide hairpin monomers incorporated into the structure is less than about 3, 4, 5, 6, 7, 8, 9, 10, 11, 12, 13, 14, 15, 20, 30, 40, 50, 60, 70, 80, 90, or 100 monomers. In various embodiments, the number of oligonucleotide hairpin monomers incorporated into the structure is about 2 to 100, 2 to 50, 10 to 50, 2 to 50, 5 to 30, 2 to 15, 2 to 14, 2 to 10, 3 to 15, 3 to 14, 3 to 10, 4 to 15, 4 to 14, 4 to 10, 5 to 15, 5 to 14, 5 to 10, 6 to 15, 6 to 14, 6 to 10, 7 to 15, 7 to 14, 7 to 10, 3 to 6, 3 to 7, 3 to 8, 3 to 9, 4 to 6, 4 to 7, 4 to 8, or 4 to 9 monomers. In various embodiments, the dispersity of a plurality of structures is from about 1 to about 2.

In some embodiments, a plurality of first oligonucleotide hairpin monomers, initiator oligonucleotides, and second oligonucleotide hairpin monomers are contacted. In some embodiments, control of HCR is exerted by using particular ratios of oligonucleotide hairpin monomers to initiator oligonucleotides. In some embodiments, the ratio of the first oligonucleotide hairpin monomer to the initiator oligonucleotide is from about 1:1 to about 50:1. In further embodiments, the ratio of the first oligonucleotide hairpin monomer to the initiator oligonucleotide is about 1:1, 2:1, 4:1, 6:1, 8:1, or 10:1. In some embodiments, the ratio of the second oligonucleotide hairpin monomer to the initiator oligonucleotide is from about 1:1 to about 50:1. In some embodiments, the ratio of the second oligonucleotide hairpin monomer to the initiator oligonucleotide is about 1:1, 2:1, 4:1, 6:1, 8:1, or 10:1.

In some embodiments, the structure is detected by gel electrophoresis, mass spectrometry, light scattering spectroscopy, colorimetry, fluorescent microscopy, fluorescent spectroscopy, electron microscopy, atomic force microscopy, nuclear magnetic resonance (NMR) depending or a combination thereof.

### Oligonucleotides

Oligonucleotides useful in the materials and methods of the disclosure include DNA oligonucleotides, RNA oligonucleotides, modified forms thereof, or a combination thereof. In any aspects or embodiments described herein, an oligonucleotide is single-stranded, double-stranded, or partially double-stranded. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkages of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—COO, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C₂ to C10 alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

### Additional Moieties

The disclosure contemplates that, in various embodiments, an oligonucleotide hairpin monomer further comprises an additional moiety. In some embodiments, an initiator oligonucleotide further comprises an additional moiety. In methods comprising multiple oligonucleotide hairpin monomers, it is contemplated that one or more of the oligonucleotide hairpin monomers comprises an additional moiety. In some embodiments, all oligonucleotide hairpin monomers in a reaction comprise an additional moiety. Any combination of oligonucleotide hairpin monomers and initiator oligonucleotides in a reaction may further comprise an additional moiety, and the additional moieties may be either all the same or one or more of the additional moieties is different.

In various embodiments, the additional moiety is an aptamer, a protein (e.g., a therapeutic protein), a peptide, a nanoparticle, a quantum dot, a small molecule, a detectable marker, or a combination thereof. In some embodiments, the protein is an antibody. An aptamer is an oligonucleotide sequence that can be evolved to bind to various target analytes of interest (e.g., a protein). The term “small molecule,” as used herein, refers to a chemical compound, or any other low molecular weight organic compound, either natural or synthetic. By “low molecular weight” is meant compounds having a molecular weight of less than 1500 Daltons, typically between 100 and 700 Daltons.

As described above, attaching proteins (e.g., an antibody) to monomers and/or to initiator oligonucleotide(s) is also contemplated by the disclosure. In general, it is difficult to oligomerize proteins together, but methods of the disclosure can be used to generate orthogonal arrangements of proteins (e.g., antibodies). Such arrangements can be used, e.g., to study the cooperativity and therapeutic efficacy of a plurality of antibodies.

In some embodiments, the detectable marker is a fluorophore. In various embodiments, the fluorophore is fluorescein derivatives, rhodamine derivatives, cyanine dyes, AlexaFluor dyes, ATTO dyes.

Nanoparticles. Methods of the disclosure include those in which one or more oligonucleotide hairpin monomers and/or initiator oligonucleotide(s) are attached or associated with a nanoparticle. In some embodiments, the methods result in oligomerization of nanoparticles or quantum dots. In general, nanoparticles contemplated by the disclosure include, for example and without limitation, a protein, a metal, a semiconductor, a liposomal particle, a polymer-based particle (e.g., a poly (lactic-co-glycolic acid) (PLGA) particle), insulator particle compositions, and a dendrimer (organic versus inorganic). Thus, in various embodiments, the nanoparticle is organic (e.g., a liposome), inorganic (e.g., gold, silver, or platinum), porous (e.g., silica-based or metal organic-framework-based), or hollow.

Thus, the disclosure contemplates nanoparticles that comprise a variety of inorganic materials including, but not limited to, metals, semi-conductor materials or ceramics as described in U.S. Patent Publication No 20030147966. For example, metal-based nanoparticles include those described herein. In various embodiments, the nanoparticle is metallic, a semiconductor, an insulator, an upconverting core, a micelle, a dendrimer, a liposome, a polymer, a metal-organic framework, a protein, or a combination thereof. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), chitosan, or a related structure. In some embodiments, the polymer is poly(lactic-co-glycolic acid) (PLGA).

Liposomal particles, for example as disclosed in International Patent Application No. PCT/US2014/068429 (incorporated by reference herein in its entirety) are also contemplated by the disclosure. Hollow particles, for example as described in U.S. Patent Publication Number 2012/0282186 (incorporated by reference herein in its entirety) are also contemplated herein. Liposomes of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, or a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Oligonucleotides contemplated for use in the methods include those attached to a nanoparticle through any means (e.g., covalent or non-covalent attachment). Regardless of the means by which the oligonucleotide is attached to the nanoparticle, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in PCT/US2014/068429, which is incorporated by reference herein in its entirety. Methods of attaching oligonucleotides to a protein are described, e.g., in U.S. Patent Application Publication No. 2017/0232109 and Brodin et al., J Am Chem Soc. 137(47): 14838-41 (2015), which are incorporated by reference herein in their entirety.

### Methods of Detecting an Analyte

In any of the aspects or embodiments of the disclosure, methods for controlling the hybridization chain reaction are provided, allowing for the creation of structures having defined numbers of monomers. The ability to control the number of monomers in a structure is advantageous for visualizing analytes (e.g., RNA) in cells. For example, an oligonucleotide hairpin monomer comprising a fluorophore and quencher where fluorescence is turned off, will lead to turn on and amplification of the fluorophore via HCR. However, if the number of monomers in a formed structure is unknown then quantitation of the amount of analyte in a sample is problematic. By controlling the number of monomers that are incorporated in a structure via HCR, structures with known numbers of monomers are created and the fluorescence of those known structures can then be used to quantitate the amount of an analyte in a sample. For example and without limitation, a calibration curve of fluorescence turn on of the monomers versus initiator concentration can be constructed. To calculate the amount of analyte (e.g., mRNA, miRNA, siRNA) in an unknown sample, the sample can be treated with a determined number of monomers. Then, the unknown amount of analyte (i.e., initiator) can be calculated according to the fluorescence turn on when compared to the calibration curve.

In some aspects, the disclosure provides a method of detecting an analyte in a sample, the method comprising contacting the sample with a) a first oligonucleotide hairpin monomer comprising a first toehold, a first duplex stem, a first hairpin loop, and a fluorescent marker, wherein the first duplex stem comprises a base-pair mismatch at a position that is proximal to the first toehold and distal to the first hairpin loop, relative to a midpoint of the first duplex stem; b) an initiator oligonucleotide; and c) a second oligonucleotide hairpin monomer comprising a second toehold, a second duplex stem, and a second hairpin loop; wherein one or more of the first duplex stem, the first toehold, and the initiator oligonucleotide comprises an analyte-binding region; and measuring fluorescence of the sample after the contacting to detect the analyte. In some embodiments, the initiator oligonucleotide comprises the analyte-binding region. In some embodiments, the first oligonucleotide hairpin monomer comprises the analyte-binding region. In some embodiments, the second oligonucleotide hairpin monomer comprises the analyte-binding region. In some embodiments, the initiator oligonucleotide, first oligonucleotide hairpin monomer, and second oligonucleotide hairpin monomer each comprise an analyte-binding region.

In further aspects, the disclosure provides a method of detecting an analyte in a sample, the method comprising contacting the sample with: a) a first oligonucleotide hairpin monomer comprising: (i) a first duplex stem comprising nucleotide sequence B and nucleotide sequence C that are substantially complementary to each other, (ii) a first hairpin loop at a first end of the first duplex stem, (iii) a toehold comprising nucleotide sequence A at a second end of the first duplex stem, and (iv) a fluorescent marker, wherein the first duplex stem comprises a base-pair mismatch at a position that is proximal to nucleotide sequence A and distal to the first hairpin loop, relative to a midpoint of the first duplex stem; b) an initiator oligonucleotide comprising nucleotide sequence E that is substantially complementary to nucleotide sequence B of the first oligonucleotide hairpin monomer; and c) a second oligonucleotide hairpin monomer comprising: (i) a second duplex stem comprising nucleotide sequence G and nucleotide sequence H that are substantially complementary to each other, (ii) a second hairpin loop at a first end of the second duplex stem that is substantially complementary to nucleotide sequence A of the first oligonucleotide hairpin monomer, and (iii) a toehold comprising nucleotide sequence F at a second end of the second duplex stem that is substantially complementary to the first hairpin loop of the first oligonucleotide hairpin monomer, wherein one or more of the initiator oligonucleotide, nucleotide sequence B, and nucleotide sequence A comprise an analyte-binding region; and measuring fluorescence of the sample after the contacting to detect the analyte. In some embodiments, the initiator oligonucleotide comprises the analyte-binding region. In some embodiments, the first oligonucleotide hairpin monomer comprises the analyte-binding region. In some embodiments, the second oligonucleotide hairpin monomer comprises the analyte-binding region.

In some embodiments, the analyte is a nucleic acid. In further embodiments, the analyte is DNA, RNA, a protein, a peptide, or a combination thereof. In some embodiments, the analyte is messenger RNA (mRNA), microRNA (miRNA), small interfering RNA (siRNA), or a combination thereof. In some embodiments, the analyte-binding region is substantially complementary to the nucleic acid. In some embodiments, the second oligonucleotide hairpin monomer comprises an additional fluorescent marker. In further embodiments, the fluorescent marker and the additional fluorescent marker are different. In some embodiments, the analyte-binding region comprises an antibody. In some embodiments, the sample is contacted with a plurality of first oligonucleotide hairpin monomers, initiator oligonucleotides, and/or second oligonucleotide hairpin monomers.

### REFERENCES

1. Wang, F.; Willner, B.; Willner, I., DNA nanotechnology with one-dimensional self-assembled nanostructures. Curr Opin Biotechnol 2013, 24 (4), 562-74.
2. Dirks, R. M.; Pierce, N. A., Triggered amplification by hybridization chain reaction. Proc Natl Acad Sci USA 2004, 101 (43), 15275-8.
3. Choi, H. M.; Beck, V. A.; Pierce, N. A., Next-generation in situ hybridization chain reaction: higher gain, lower cost, greater durability. ACS Nano 2014, 8 (5), 4284-94.
4. Ang, Y. S.; Yung, L. Y., Rational design of hybridization chain reaction monomers for robust signal amplification. Chem Commun (Camb) 2016, 52 (22), 4219-22.
5. Idili, A.; Porchetta, A.; Amodio, A.; Vallee-Belisle, A.; Ricci, F., Controlling Hybridization Chain Reactions with pH. Nano Lett 2015, 15 (8), 5539-44.
6. Samanta, D.; Ebrahimi, S. B.; Mirkin, C. A., Nucleic-Acid Structures as Intracellular Probes for Live Cells. Adv Mater 2019, e1901743.
7. Wu, Z.; Liu, G. Q.; Yang, X. L.; Jiang, J. H., Electrostatic nucleic acid nanoassembly enables hybridization chain reaction in living cells for ultrasensitive mRNA imaging. J Am Chem Soc 2015, 137 (21), 6829-36.
8. Venkataraman, S.; Dirks, R. M.; Rothemund, P. W.; Winfree, E.; Pierce, N. A., An autonomous polymerization motor powered by DNA hybridization. Nat Nanotechnol 2007, 2 (8), 490-4.
9. Meng, W.; Muscat, R. A.; McKee, M. L.; Milnes, P. J.; EI-Sagheer, A. H.; Bath, J.; Davis, B. G.; Brown, T.; O'Reilly, R. K.; Turberfield, A. J., An autonomous molecular assembler for programmable chemical synthesis. Nat Chem 2016, 8 (6), 542-8.
10. Li, Z.; He, X.; Luo, X.; Wang, L.; Ma, N., DNA-Programmed Quantum Dot Polymerization for Ultrasensitive Molecular Imaging of Cancer Cells. Anal Chem 2016, 88 (19), 9355-9358.
11. Jia, F.; Wang, D. L.; Lu, X. G.; Tan, X. Y.; Wang, Y. Y.; Lu, H.; Zhang, K., Improving the Enzymatic Stability and the Pharmacokinetics of Oligonucleotides via DNA-Backboned Bottlebrush Polymers. Nano Letters 2018, 18 (11), 7378-7382.
12. McMillan, J. R.; Hayes, O. G.; Remis, J. P.; Mirkin, C. A., Programming Protein Polymerization with DNA. J Am Chem Soc 2018, 140 (46), 15950-15956.
13. Ikuta, S.; Takagi, K.; Wallace, R. B.; Itakura, K., Dissociation kinetics of 19 base paired oligonucleotide-DNA duplexes containing different single mismatched base pairs. Nucleic Acids Res 1987, 15 (2), 797-811.
14. Bhattacharya, P. K.; Cha, J.; Barton, J. K., 1H NMR determination of base-pair lifetimes in oligonucleotides containing single base mismatches. Nucleic Acids Res 2002, 30 (21), 4740-50.
15. Srinivas, N.; Ouldridge, T. E.; Sulc, P.; Schaeffer, J. M.; Yurke, B.; Louis, A. A.; Doye, J. P.; Winfree, E., On the biophysics and kinetics of toehold-mediated DNA strand displacement. Nucleic Acids Res 2013, 41 (22), 10641-58.
16. Zhang, D. Y.; Winfree, E., Control of DNA strand displacement kinetics using toehold exchange. J Am Chem Soc 2009, 131 (47), 17303-14.
17. Broadwater, D. W. B., Jr.; Kim, H. D., The Effect of Basepair Mismatch on DNA Strand Displacement. Biophys J 2016, 110 (7), 1476-1484.
18. Panyutin, I. G.; Hsieh, P., Formation of a single base mismatch impedes spontaneous DNA branch migration. J Mol Biol 1993, 230 (2), 413-24.
19. Li, C.; Li, Y.; Xu, X.; Wang, X.; Chen, Y.; Yang, X.; Liu, F.; Li, N., Fast and quantitative differentiation of single-base mismatched DNA by initial reaction rate of catalytic hairpin assembly. Biosens Bioelectron 2014, 60, 57-63.
20. Machinek, R. R.; Ouldridge, T. E.; Haley, N. E.; Bath, J.; Turberfield, A. J., Programmable energy landscapes for kinetic control of DNA strand displacement. Nat Commun 2014, 5, 5324.
21. Hayes, O. G.; McMillan, J. R.; Lee, B.; Mirkin, C. A., DNA-Encoded Protein Janus Nanoparticles. J. Am. Chem. Soc. 2018, 140, 9269-9274.
22. McMillan, J. R.; Hayes, O. G.; Remis, J. P.; Mirkin, C. A., Programming Protein Polymerization with DNA. J. Am. Chem. Soc. 2018, 140, 15950-15956.
23. Chen, J.; Tsai, A.; Petrov, A.; Puglisi, J. D., Nonfluorescent quenchers to correlate single-molecule conformational and compositional dynamics. J. Am. Chem. Soc. 2012, 134, 5734-5737.

### EXAMPLES

### Example 1

As proof-of-concept, hairpin monomers with a 6 base-pair (bp) toehold, 18 bp complementary stem, and 6 bp loop were studied. Mismatches were then introduced at either the 2, 4, 6, or 8 position of the stem. Reactions were set up in PBS for 4 hours using stoichiometric amounts of initiator (I1), hairpin 1 (H1), and hairpin 2 (H2) at 5 uM per sequence to target a theoretical DP=2. The product distributions were then characterized via agarose gel electrophoresis to calculate number average DP, weight average DP, and dispersity using ImageJ densitometry analysis. While initiating H1 with no mismatch (Table 1, entry 1) showed a number-average DP close to theoretical (2.6), only 43% conversion and Ð=3.0 were observed. Furthermore, reactions where the hairpins contained a mismatch at positions 2, 4, and 6 also led to uncontrolled polymerizations with high dispersities. Interestingly, when a mismatch was placed at position 8, a DP of 2.3 and dispersity of 1.4 was observed, suggesting the mismatch was modifying the polymerization kinetics.

Although hairpin 1 (H1) and hairpin 2 (H2) are complementary to each other, the metastable hairpins vary in their local G-C content. Specifically, H1 contains 60% G-C content in the first 10 bases of the stem while H2 contains 40%. Since G-C bps fray up to 10× slower than A-T, the different local G-C content should also affect strand displacement and resultant polymerization products. Therefore, reactions using an initiator that could open H2 (12) were also studied.

TABLE 1
Results according to mismatch position obtained from gel electrophoresis
analysis of reactions using stoichiometric amounts of initiator:H1:H2 to
target a degree of polymerization = 2.

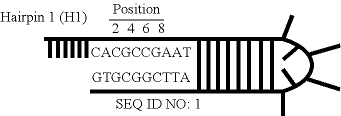

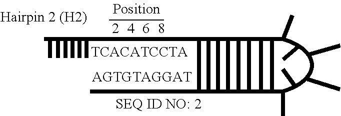

| | INITIATING | MISMATCH | % | | | |
|---|---|---|---|---|---|---|
| ENTRY | MONOMER^{a} | POSITION | CONVERSION | DP_{N} | DP_{W} | Ð |
| 1 | H1 | NONE | 43 | 2.6 | 7.7 | 3.0 |
| 2 | | 2 | 23 | 0.36 | 2.2 | 6.1 |
| 3 | | 4 (HIM) | 67 | 2.5 | 5.8 | 2.3 |
| 4 | | 6 | 72 | 2.0 | 4.5 | 2.2 |
| 5 | | 8 | 89 | 1.9 | 2.8 | 1.4 |
| 6 | H2 | NONE | 82 | 4.5 | 7.5 | 1.7 |
| 7 | | 2 | 47 | 0.80 | 1.8 | 2.3 |
| 8 | | 4 (H2M) | 96 | 2.3 | 2.8 | 1.2 |
| 9 | | 6 | 97 | 2.5 | 2.9 | 1.2 |
| 10 | | 8 | 70 | 1.4 | 2.8 | 2.0 |

^{a}Reactions were run for 4 hours in PBS at a concentration of 5 uM per DNA strand.

Interestingly, initiating H2 with no mismatches instead of H1 improved number-average DP dispersity to 1.7, but experimental DP was over twice as high as theoretical DP (Table 1, entry 6). These results suggested that the G-C content of the monomers could affect the polymerization, but the poor control obscures any conclusions. When H2 was initiated for monomer designs containing either the 4 (Table 1, entry 8) or 6 position (Table 1, entry 9) mismatches, experimental DPs (2.3 for position 4, 2.5 for position 6) closely matched theoretical values and low dispersities were observed.

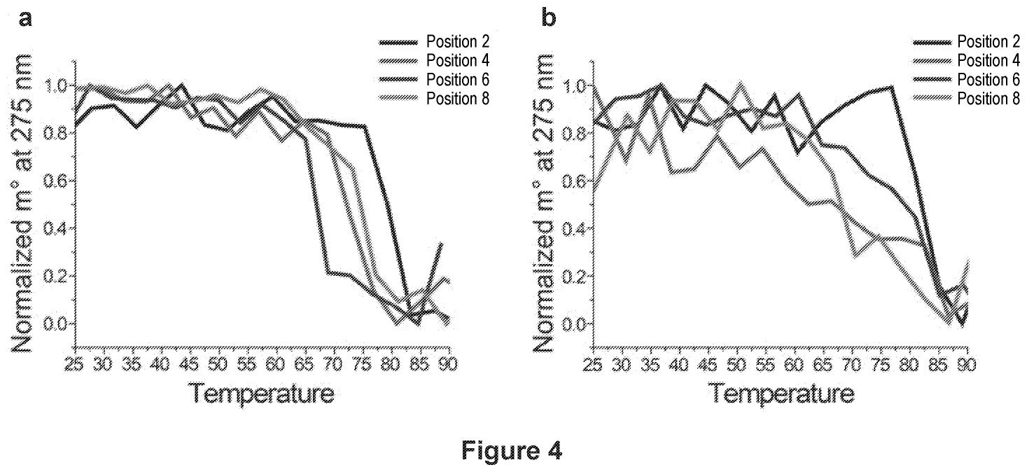

Melting experiments of the hairpins were then conducted using CD to understand how the mismatches were affecting the duplexes (Table 2, FIG. 4). Decreased melting temperatures were observed for all mismatch positions, with positions 4, 6, and 8 showing an earlier onset of melting than position 2 or no mismatch. Together, these results suggest that the addition of a mismatch at positions 4, 6, and 8 leads to less stable duplexes. This led us to the conclusion that the mismatches were leading to increased fraying of the base pairs in the stem, effectively increasing the length of the toehold. The increased length of the toehold presumably increases the rate of strand displacement for both initiation and propagation steps. Since the stem portion of the hairpin dictates the metastability of the hairpins, we continued to use hairpins with the mismatch at the 4 position (denoted at H1m and H2m) to avoid any background polymerization.

TABLE 2
Melting temperatures of DNA hairpins with different mismatch
positions. Determined by taking a CD measurement every
2° C. from 25-90° C. at a temperature ramp rate
of 0.1° C./min, normalizing the m° at 275 nm,
and taking the temperature at the 0.5 y-axis value after normalization.

| | PREDICTED | EXPERIMENTAL |
|---|---|---|
| | MELTING | MELTING |
| NAME | TEMPERATURE | TEMPERATURE |
| H1 | 78.4 | 79 |
| H1 (POS 2) | 79.4 | 79 |
| H1 (POS 4) | 72.6 | 74 |
| H1 (POS 6) | 68.1 | 67 |
| H1 (POS 8) | 75.2 | 75 |
| H2 | 81 | 81 |
| H2 (POS 2) | 80.2 | 82 |
| H2 (POS 4) | 80.8 | 62 |
| H2 (POS 6) | 77.6 | 78 |
| H2 (POS 8) | 75.2 | 66 |

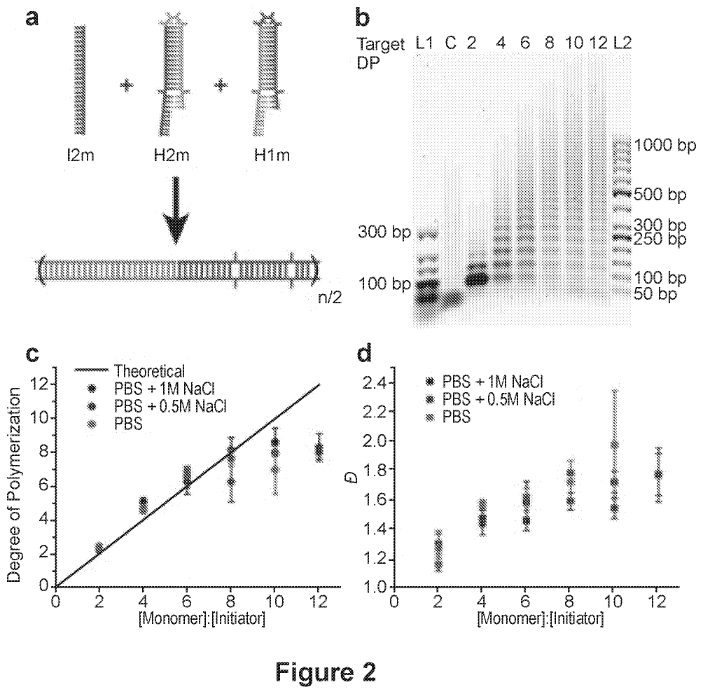

Next, different degrees of polymerization were targeted when initiating H2m using the corresponding initiator (I2m, FIG. 2). Reactions were set up targeting DPs of 2, 4, 6, 8, and 10. Kinetic analysis of the reactions at 4, 6, and 16 hours showed that higher DP polymerizations (DP>6) required at least 16 hours to reach >90% conversion. SEC analysis of the resultant polymers showed a shift to lower retention times with higher DPs, but polymerizations quickly reached the exclusion limit of the columns. Accordingly, the polymerizations were analyzed using agarose gel electrophoresis (3%). Polymer distributions shifted to higher molecular weights according to monomer:initiator ratios, suggesting that the molecular weights were dictated by initiator concentration in accordance with previous literature. However, upon gel densitometry analysis using ImageJ software to attain Mn, Mw, and D, experimental DPs closely followed theoretical DPs at high conversions up to a DP of 8-10 until a decrease in gel resolution inhibited analysis. These results represented the first time that DNA polymerization via HCR resulted in predictable molecular weights according to initial M:I ratio. Polymer dispersity remained between 1.3-1.8, further suggesting that the polymerization was controlled.

Upon an increase in salt concentration in the PBS with an additional 0.5 M NaCl or 1 M NaCl, polymerizations at room temperature yielded polymerizations with lower dispersity at DPs greater than 4 compared to PBS conditions. Additionally, a DP of 12 was reliably achieved under the high salt concentrations, but higher DPs resulted in low molecular weights and incomplete conversions over a 16 hour timescale. The dispersity at DP=2 lead us to believe that the higher salt conditions were affecting the fraying of the hairpins between the mismatch position and the toehold, effectively raising the melting temperature of that region and decreases the effect of increased initiation from the increased toehold length. The increase in melting temperature of the bases between the mismatch and toehold we confirmed with CD spectroscopy, which suggested a melting temperature of approximately 30 C. Conducting polymerizations at 37 C reinstated the monodispersity of DP=2, similar to polymerizations conducted in PBS, but the salt concentration and increased temperature decreased the dispersity obtained in polymer samples down to 1.3-1.4, well within the rage of molecular weight dispersity in which a polymerization is considered controlled.

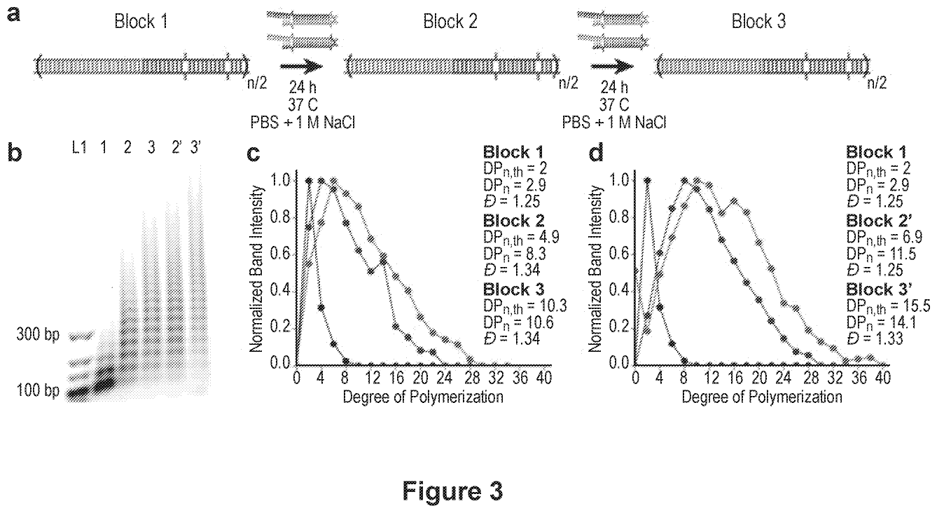

To determine if low dispersity and predictable molecular weights are retained after monomer addition or only present after the initial polymerization, two chain extensions were performed (FIG. 3a). Starting with a first block with a DP=2.9 (Ð=1.25), H1m and H2m were added to target an overall DP=4.9 for Block 1 and Block 2. After incubating at 37° C. for 24 hours, additional H1m and H2m were added to target a total theoretical DP=10.3 for Block 1, Block 2, and Block 3. Efficient chain extensions were observed using densitometry analysis of a 1% agarose gel (FIG. 3b). Average DP was observed to shift according to monomer additions and a final experimental DP=10.6 matched well with the theoretical DP=10.6 and dispersity only increased to 1.34 (FIG. 3c). In a separate chain extension, Block 1 was observed to again be quantitatively oligomerized to higher DPs with a final DP of 14.1 being the highest that was observed using the controlled HCR technique disclosed herein while maintaining Ð=1.3 (FIG. 3d). These results confirmed that controlled HCR polymerizations are inherent to the designed DNA monomers, independent of salt concentration, and is retained even after two monomer additions.

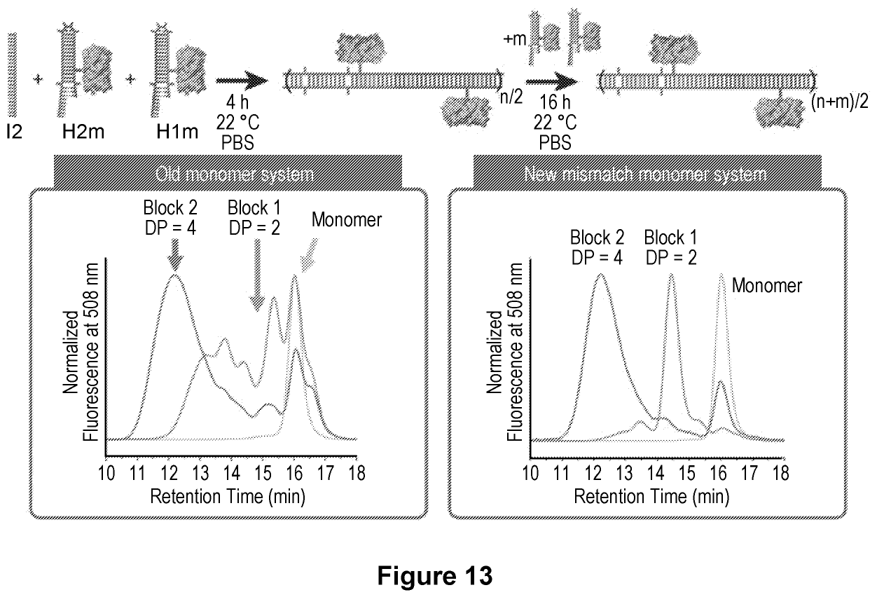

Finally, the new hairpin design was conjugated to a GFP protein containing a single surface cysteine to evaluate whether the polymerization control translates when cargo such as proteins are attached to the DNA hairpins. Homopolymerization of GFP lead to analogous control over the polymer products as the DNA only system (FIG. 13). Notably, higher salt concentrations were required to achieve high conversion of protein polymers targeting higher DPs. Oligo(GFP) was successfully quantitatively chain-extended, as shown in the SEC trances of the fluorescence of GFP and global shift of polymer distributions observed after gel electrophoresis.

Well-defined DNA polymers using HCR are disclosed herein. Predictable molecular weights, low dispersities, and controlled chain extensions demonstrate that initiation is effectively decoupled from and occurs faster than propagation when a mismatch is present. This discovery warrants investigation into achieving higher DPs, low concentration polymerizations, and responsive multiblock DNA polymers. The results provided herein, and the concept of controlling HCR, leads to powerful changes in current applications such as biosensing, as a direct comparison between initiator, monomer concentration, and signal output can be realized. Therefore, by understanding how hairpin design affects HCR polymerization, a new DNA-mediated controlled polymerization approach is disclosed herein.

As shown herein, control over DNA oligomerization was achieved through the introduction of a base-pair mismatch in the duplex of the metastable hairpin monomers. The mismatch modification allows one to energetically differentiate initiation versus propagation events, leading to DNA oligomers up to 10-mers with degree of polymerization (DP) dispersity between 1.3 and 1.6. Importantly, even after two consecutive chain extensions from DP=2 to a final DP=14, dispersity remains unaffected, showing that well-defined block copolymers can be achieved. Taken together, this work defines an effective method for controlling HCR polymerization with macromolecules in a manner analogous to the controlled polymerization of small molecules.

Base-pair mismatches were introduced at specific locations (2, 4, 6, or 8) in the duplex of the DNA monomers during synthesis, and the initiating strand is designed to be complementary to the first monomer. For the studied system, the mismatch caused increased fraying of the adjacent complementary base pairs, effectively increasing the length of the toehold while maintaining monomer stability. The mismatch is then translated to propagation events, slowing strand displacement. Both effects should differentiate initiation and propagation energies, and the mismatch at position 4 led to the best oligomerization control. By introducing base pair mismatches into previously studied systems, it is demonstrated herein that polymerization products can be predicted from the ratios of reagents added.

TABLE 3
DNA sequences, molecular weights, and extinction coefficients.

| NAME | SEQ. ID NO: | SEQUENCE (5′→3′) | ε₂₆₀ (M⁻¹CM⁻¹) |
|---|---|---|---|
| H1 | 1 | TTA ACC CAC GCC GAA TCC TAG ACT CAA AGT AGT | 463800 |
| | | CTA GGA TTC GGC GTG | |
| H2 | 2 | AGT CTA GGA TTC GGC GTG GGT TAA CAC GCC GAA | 461500 |
| | | TCC TAG ACT ACT TTG | |
| H1 POS 2 | 3 | TTA ACC CAC GCC GAA TCC TAG ACT CAA AGT AGT | 468300 |
| | | CTA GGA TTC GGC GAG | |
| H2 POS 2 | 4 | ACT CTA GGA UC GGC GTG GGT TAA CAC GCC GAA | 457000 |
| | | TCC TAG ACT ACT TTG | |
| H1 POS 4 | 5 | TTA ACC CAC GCC GAA TCC TAG ACT CAA AGT AGT | 471200 |
| (H1M) | | CTA GGA TTC GGG GTG | |
| H2 POS 4 | 6 | AGT CTA GGA TTC GGC GTG GGT TAA CAC GCC GAA | 457800 |
| (H2M) | | TCC TAC ACT ACT TTG | |
| H1 POS 6 | 7 | TTA ACC CAC GCC GAA TCC TAG ACT CAA AGT AGT | 464800 |
| | | CTA GGA TTC CGC GTG | |
| H2 POS 6 | 8 | AGT CTT GGA UC GGC GTG GGT TAA CAC GCC GAA | 455600 |
| | | TCC TAG ACT ACT TTG | |
| H1 POS 8 | 9 | TTA ACC CAC GCC GAA TCC TAG ACT CAA AGT AGT | 468700 |
| | | CTA GGA TAC GGC GTG | |
| H2 POS 8 | 10 | AGT CTA GCA UC GGC GTG GGT TAA CAC GCC | 457600 |
| | | GAA TCC TAG ACT ACT TTG | |
| I | 11 | AGT CTA GGA TTC GGC GTG GGT TAA | 239600 |
| I2 | 12 | CAA AGT AGT CTA GGA TTC GGC GTG | 238700 |
| I2B | 13 | CAA AGT AGT GTA GGA UC GGC GTG | 242200 |

DNA monomer preparation. Following DNA purification, each monomer was diluted in PBS with the appropriate added salt concentration (either 0, additional 0.5 M NaCl, or additional 1 M NaCl) to a concentration of 20 μM. The solution of each DNA was then snapcooled by heating to 95° C. for 4 min, cooled to 4° C. over 1 minute held there for 4 minutes, then brought to room temperature. The concentration was then confirmed using the extinction coefficient of the DNA at 260 nm.

DNA oligomerizations at 22° C. Polymerizations were conducted at 5 μM per monomer. For example, for a polymerization targeting a DP=4, 13.5 μL of 18.4 μM H1 m, 13.2 μL of 18.9 μM H2m, and 20.9 μL PBS were added to an Eppendorf tube. Then, 2.51 μL of 49.2 μM I2B was added and the polymerization was allowed to react for 16 hours before an aliquot was taken for agarose gel electrophoresis analysis.

DNA oligomerization at 37° C. Polymerizations were conducted at 5 μM per monomer. For example, for a polymerization targeting a DP=4, 13.5 μL of 18.4 μM H1m, 13.2 μL of 18.9 μM H2m, and 20.9 μL PBS+1 M NaCl were added to an Eppendorf tube. H1m and H2m were equilibrated for 10 minutes at temperature prior to addition of I2B. Then, 2.51 μL of 49.2 μM I2B was added and the polymerization was allowed to react for 16 hours before an aliquot was taken for agarose gel electrophoresis analysis.

### Example 2

Example 2 includes experiments that were described in Example 1, herein, and also includes additional experiments.

As described herein, the disclosure is generally directed to methods for controlling the oligomerization of metastable DNA hairpins using the hybridization chain reaction (HCR). This example demonstrated that control was achieved through the introduction of a base-pair mismatch in the duplex of the hairpins. The mismatch modification allowed for the kinetic differentiation of initiation versus propagation events, leading to DNA oligomers up to 10 monomers long and improving dispersities from 2.5 to 1.3-1.6. Importantly, even after two consecutive chain extensions, dispersity remained unaffected, showing that well-defined block co-oligomers can be achieved. As a proof-of-concept, this technique was then applied to hairpin monomers functionalized with a mutant green fluorescent protein to prepare protein oligomers. Taken together, this work introduced an effective method for controlling living macromolecular HCR oligomerization in a manner analogous to the controlled polymerization of small molecules.

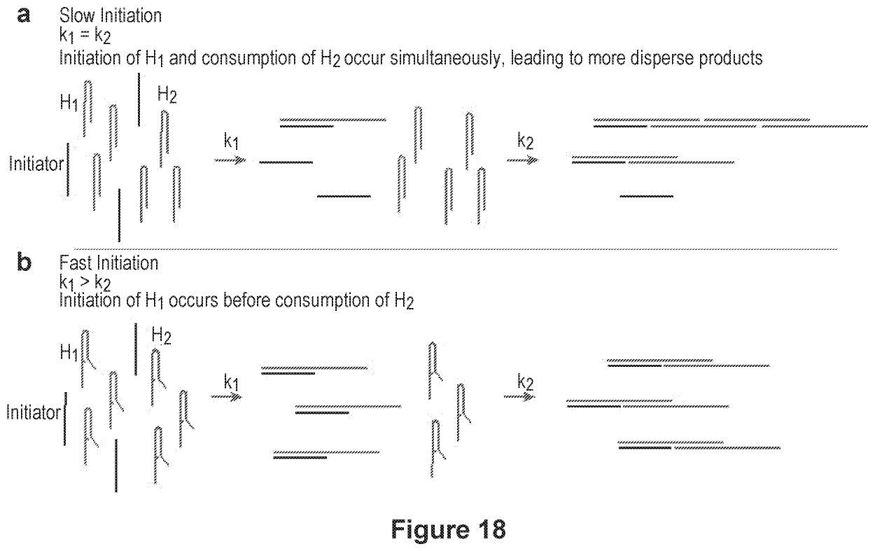

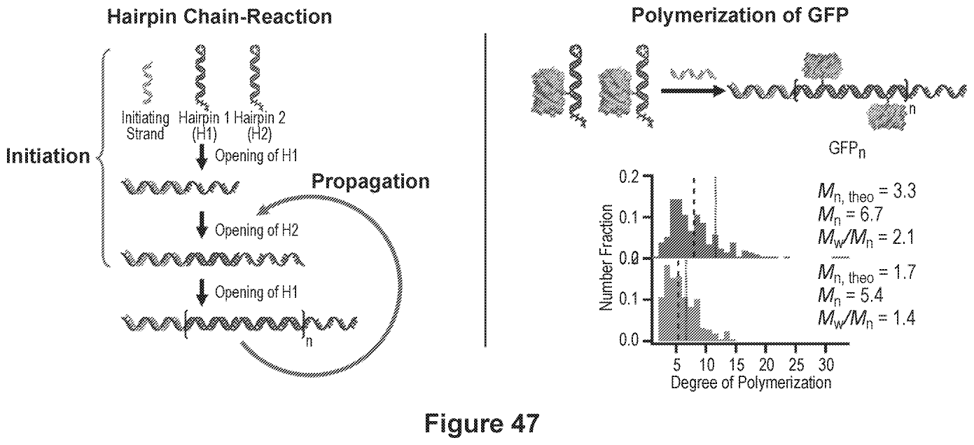

Conventional HCR oligomerization designs inherently result in high-dispersity duplexed DNA chains that contain a single-stranded DNA chain end capable of adding to additional monomers (i.e., living chain end), but do not meet the criteria for “controlled” polymerization techniques, namely predictable number of monomers per chain, low dispersity of polymer lengths, and well-defined block copolymers. In typical HCR designs, the symmetry of the hairpins means that both initiating (I1, FIG. 17) and propagating (P1, P2, FIG. 17) strands contain the same number of base pairs (bp) and undergo the same number of branch migration steps to form duplexes that are identical in length. See also FIG. 47. Thus, uncontrolled oligomerizations result from kinetically similar DNA strand displacement reactions for both initiation and propagation (FIG. 18a). Conversely, in controlled polymerizations, the rate of initiation is faster than the rate of propagation. This rate difference ensures that all of the initiating species is consumed before significant amounts of monomer are added to growing chains (FIG. 18b), resulting in a predictable DPₙ according to the amount of initiator and monomer added to the reaction.

It was contemplated that HCR oligomerization could be controlled via the introduction of a single base-pair mismatch into the duplexed stem that comprises the hairpin was explored (FIG. 14b). Two significant consequences of the mismatch lead to control. First, the mismatch increases the length of the toehold and decreases the length of the stem due to increased base-pair fraying adjacent to the mismatch [Ikuta et al., Nucleic Acids Res. 1987, 15, 797-811; Bhattacharya et al., Nucleic Acids Res. 2002, 30, 4740-4750], increasing the rate of strand displacement using a complementary sequence as an initiator (Initiation, FIG. 15b) [Srinivas et al., Nucleic Acids Res. 2013, 41, 10641-10658; Zhang et al., J. Am. Chem. Soc. 2009, 131, 17303-17314; Haley et al., bioRxiv 2018, 426668]. Second, the mismatch of each hairpin is retained during subsequent strand displacement events (Propagation, FIG. 15b), slowing down this step [Broadwater et al., Biophys. J. 2016, 110, 1476-1484; Panyutin et al., J. Mol. Biol. 1993, 230, 413-424; Li et al., Biosens. Bioelectron. 2014, 60, 57-63; Machinek et al., Nat. Commun. 2014, 5, 5324]. Both factors kinetically differentiate initiation and propagation events, leading to controlled HCR oligomerization.

As a proof of concept the most frequently employed hairpin monomers were studied, which contain a 6 bp toehold, 18 bp complementary stem, and 6 bp loop (hairpin sequence H1, hairpin sequence H2) [Dirks et al., Proc. Natl. Acad. Sci. U.S.A 2004, 101, 15275-15278]. Mismatches were then introduced at either the 2 (H1₂, H2₂), 4 (H1₄, H2₄), 6 (H1₆, H2₆), or 8 (H1₈, H2₈) positions of the 3′ end of the hairpin stem. To test whether the presence of a mismatch affects oligomer product distributions, reactions were conducted in PBS for 4 hours using stoichiometric amounts of initiator (I1) and each monomer (5 μM for each sequence) to target a DPₙ of 2 (according to the monomer:initiator ratio). If the initiation rate is increased, a molecularly pure dimer would be observed since the fast, quantitative consumption of the first hairpin monomer will inhibit subsequent oligomerization (FIG. 18b).

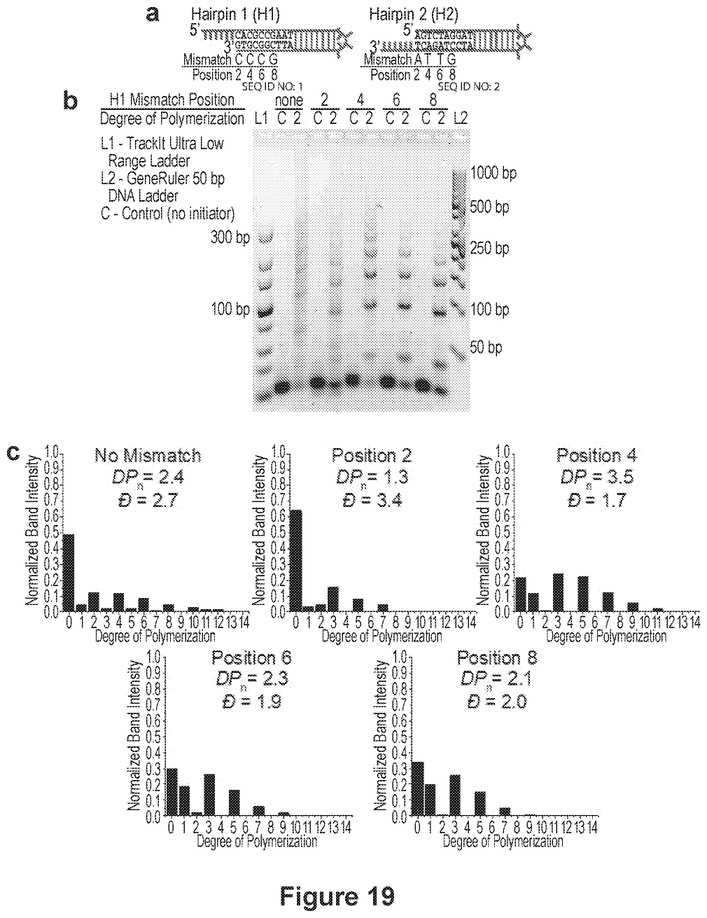

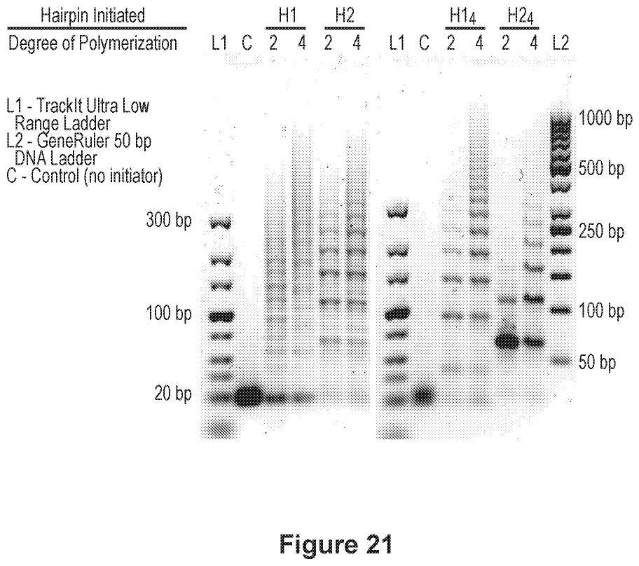

The product distributions were characterized via agarose gel electrophoresis (FIGS. 19, 20, and 21) to calculate DPₙ using ImageJ densiometry analysis (Equation 1). To understand the distribution of species formed, analogous to molecular polymerizations, a ratio of the weighted average (DP_{w}, Equation 2) to DPₙ is taken to yield a dispersity value (Ð, Equation 3), where Ð≈1 is ideal and Ð>2 is considered high dispersity. While initiating H1 with no mismatch (Table 4, Entry 1) showed a DPₙ=2.8 that is close to the expected DPₙ of 2, a Ð=2.5 was observed. Furthermore, reactions using H1₂ and H2₂ (Table 4, Entry 2), H1₄ and H2₄ (FIG. 15b, Table 4, Entry 3), or H1₆ and H2₆ (Table 4), Entry 4) also led to oligomerizations with high dispersities. Interestingly, H1₈ and H2₈ resulted in a DPₙ of 1.9, Ð of 1.6, and monomer conversion of 81% (Table 4, Entry 5), showing that the mismatch influences oligomerization kinetics in the intended manner.

$${DP}_{n} = \frac{Σ {DP}_{i} {N}_{i}}{Σ {N}_{i}} = \frac{Σ {{DP}_{i} ( Normalized Peak Intensity )}_{i}}{1} \qquad Equation 1$$

$${DP}_{w} = \frac{Σ {DP}_{i}^{2} {N}_{i}}{Σ {DP}_{i} {N}_{i}} = \frac{Σ {{DP}_{i}^{2} ( Normalized Peak Intensity )}_{i}}{Σ {DP}_{i} ( 1 )} \qquad Equation 2$$

$$Ð = \frac{{DP}_{w}}{{DP}_{n}} \qquad Equation 3$$

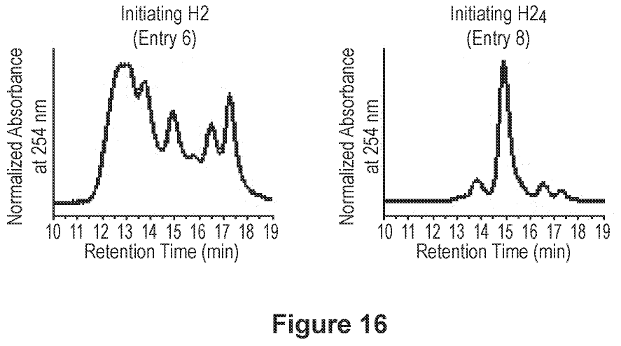

TABLE 4
Example size exclusion chromatograms (see FIG. 16) and
results from gel electrophoresis according to mismatch
position using stoichiometric amounts of initiator, H1,
and H2 to target a Degree of Polymerization = 2.

| ENTRY | INITIATING MONOMER^{A} | % CONVERSION | DP_{N} | Ð |
|---|---|---|---|---|
| 1 | H1 | 45 | 2.8 | 2.5 |
| 2 | H1₂ | 58 | 2.1 | 2.6 |
| 3 | H1₄ | 70 | 2.8 | 2.1 |
| 4 | H1₆ | 72 | 2.1 | 2.1 |
| 5 | H1₈ | 81 | 1.9 | 1.6 |
| 6 | H2 | 64 | 2.4 | 2.5 |
| 7 | H2₂ | 90 | 2.1 | 1.4 |
| 8 | H2₄ | 91 | 2.4 | 1.4 |
| 9 | H2₆ | 95 | 2.6 | 1.3 |
| 10 | H2₈ | 94 | 2.6 | 1.3 |

^{A}Subscript denotes mismatch position; reactions were run for 4 h at 22° C. in PBS, [DNA] = 5 μM per strand.

Although H1 and H2 are complementary to each other, they vary in their local G-C content. Since G-C bps fray at least 10× slower than A-T bps [Nonin et al., Biochemistry 1995, 34, 10652-10659; Kochoyan et al., Nucleic Acids Res. 1988, 16, 7685-7702; Zgarbova et al., J. Chem. Theory Comput. 2014, 1, 3177-3189], the different G-C content of the hairpin duplex affects strand displacement rates and resulting oligomerization products. Therefore, reactions were set up using initiator sequences designed to complement H2 for each monomer set (I2, I2₂, I2₄, I2₆, I2₈).

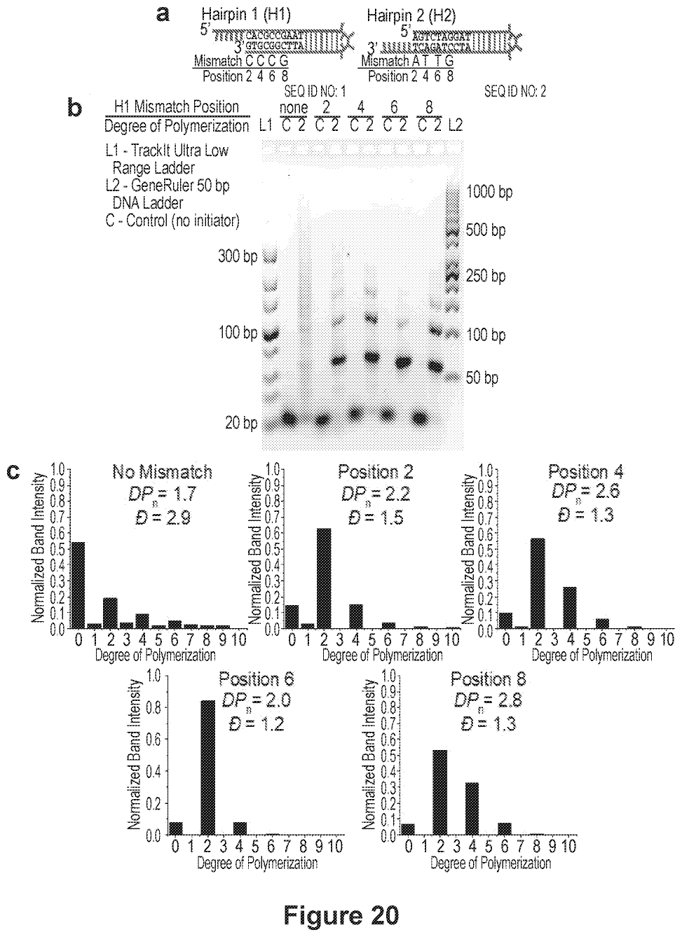

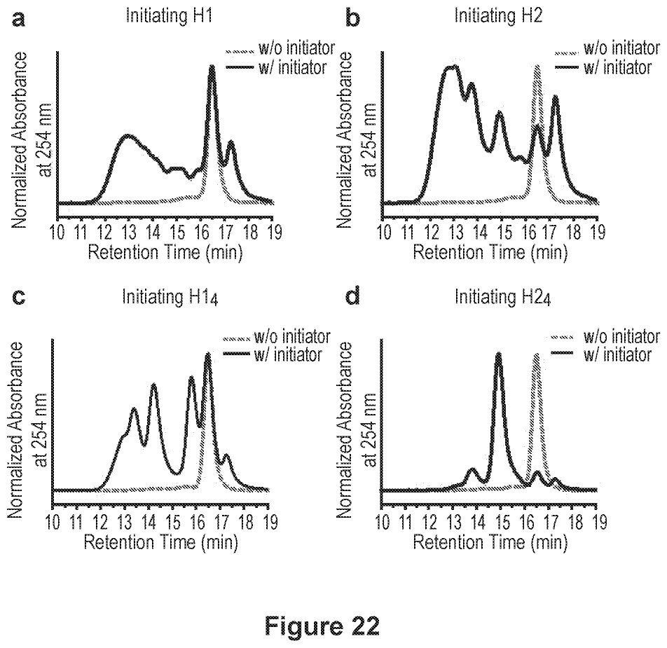

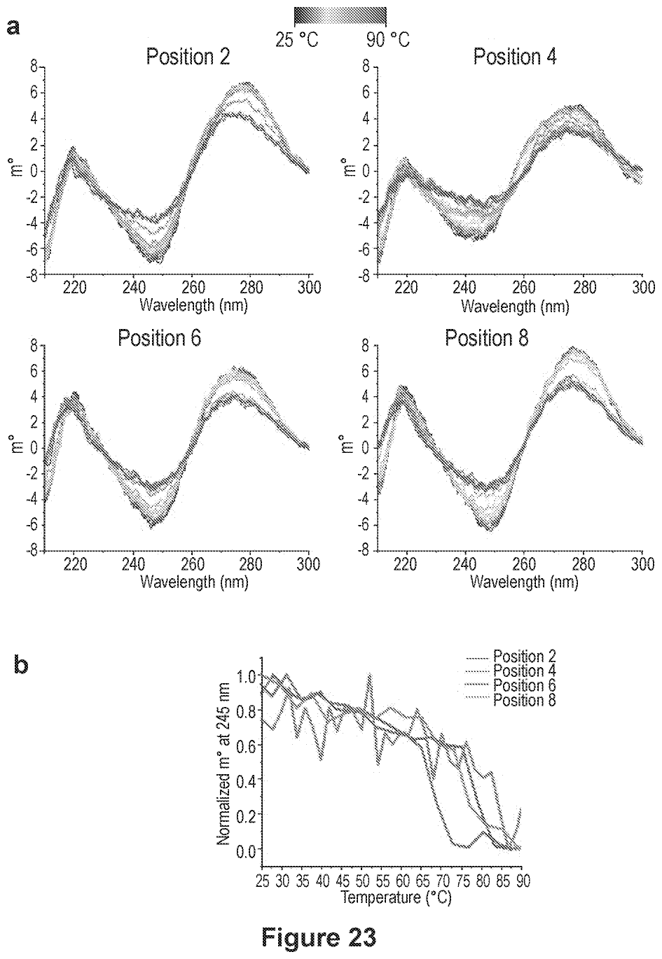

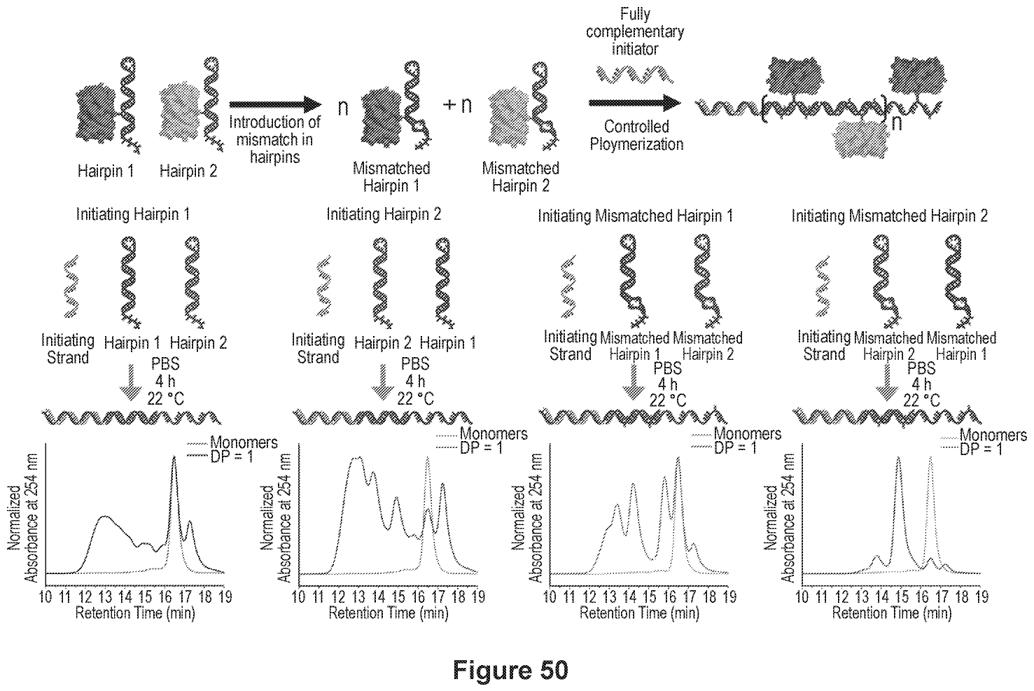

Initiating H2 with no mismatches (FIG. 15c) did not significantly change the oligomerization products (Table 4, entry 6). However, when H2 monomers containing mismatches were initiated using the respective 12, experimental DPₙ closely matched expected values, low Ð values were observed, and conversion was >90% in all cases (Table 4, Entries 7-10). The excellent agreement between expected and experimental DPₙ and Ð=1.3-1.4 strongly suggested that initiation rates were increased relative to propagation rates. Additionally, size-exclusion chromatography (SEC) analysis showed near-molecularly pure dimers when mixing I2₄, H1₄, and H2₄ (FIG. 16, FIG. 22). See also FIG. 50. Importantly, the mismatch did not significantly decrease hairpin stability since no background oligomerization was observed in the absence of initiator (FIGS. 19, 20) and melting temperatures were largely unaffected (Table 5, FIGS. 23-26).

TABLE 5
Melting Temperatures of DNA Hairpins
with Different Mismatch Positions.

| | Predicted Melting | Experimental Melting |
|---|---|---|
| Name | Temperature¹ | Temperature |
| H1 | 79 | 79 |
| H1₂ | 80 | 79 |
| H1₄ | 73 | 74 |
| H1₆ | 69 | 67 |
| H1₈ | 76 | 75 |
| H2 | 82 | 81 |
| H2₂ | 81 | 78 |
| H2₄ | 81 | 82 |
| H2₆ | 80 | 78 |
| H2₈ | 79 | 79 |

¹Predictions were calculated using IDT Oligo Analyzer 3.1 using salt conditions mimicking PBS (137 mM NaCl) and a hairpin concentration of 10 μM,

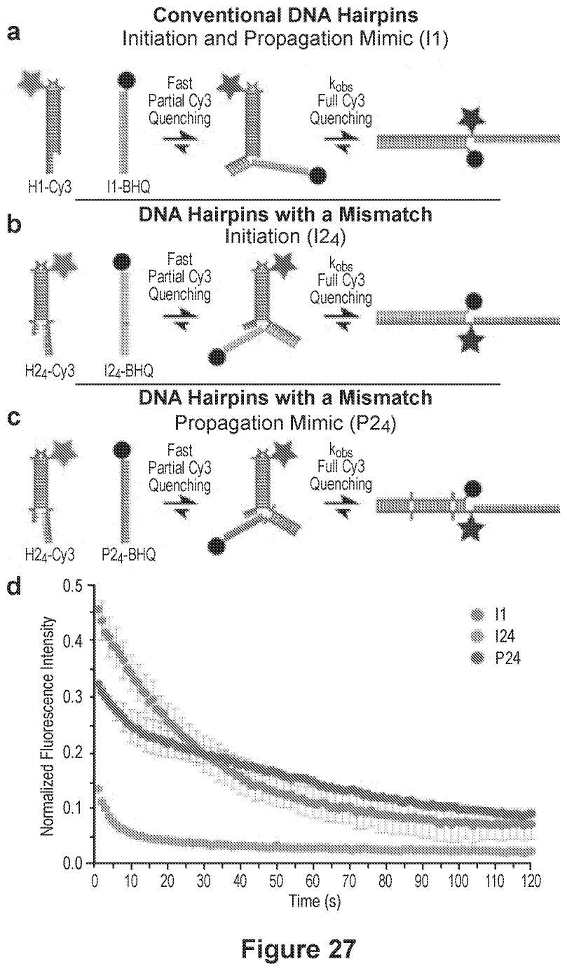

Fluorescence quenching experiments were performed to better understand the implications of the mismatch introduction on oligomerization kinetics. H1 and H2₄ hairpins were designed to contain a Cy3 fluorophore in the loop (H1-Cy3 FIG. 27a, H2₄-Cy3, FIG. 27b). Additionally, black hole quencher (BHQ) was introduced on I1 (I1-BHQ), I2₄ (I2₄-BHQ), and a strand containing the partial sequence of H1₄ to mimic the propagation step with a mismatch (P2₄-BHQ, FIG. 27c). Because I1 is identical to the propagation sequence in the conventional HCR system, I1-BHQ represents both initiating and propagating strands.

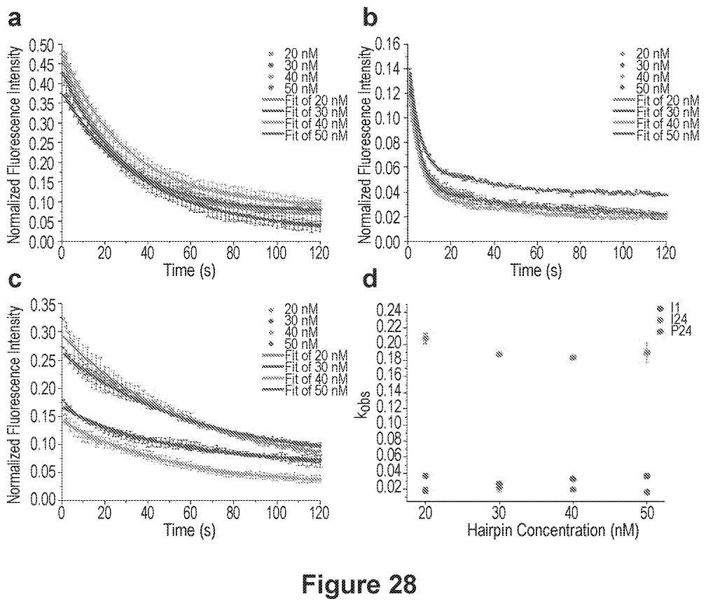

Pseudo-first order kinetics experiments revealed that each design had distinct quenching profiles (FIG. 27d, see below for details) and observed rate constants of strand displacement (k_{obs}) that were concentration independent (FIG. 28d). First, k_{obs} of H2₄-Cy3 by I2₄-BHQ (i.e., initiation) was 0.21 s⁻¹, approximately 6-times faster than the initiation in the conventional system (k_{obs}=0.037 s⁻¹). Second, the opening of H2₄-Cy3 by P2₄ (i.e., the reaction mimicking propagation) had a k_{obs}=0.017 s⁻¹, slower than the conventional and mismatch initiation strand displacement reactions. These two results confirmed that the mismatch system successfully increased the rate of initiation using a complementary strand and decreased the rate of propagation due to the presence of mismatches in the subsequent monomer additions, resulting in an overall 12-times faster initiation k_{obs} compared to propagation k_{obs}.

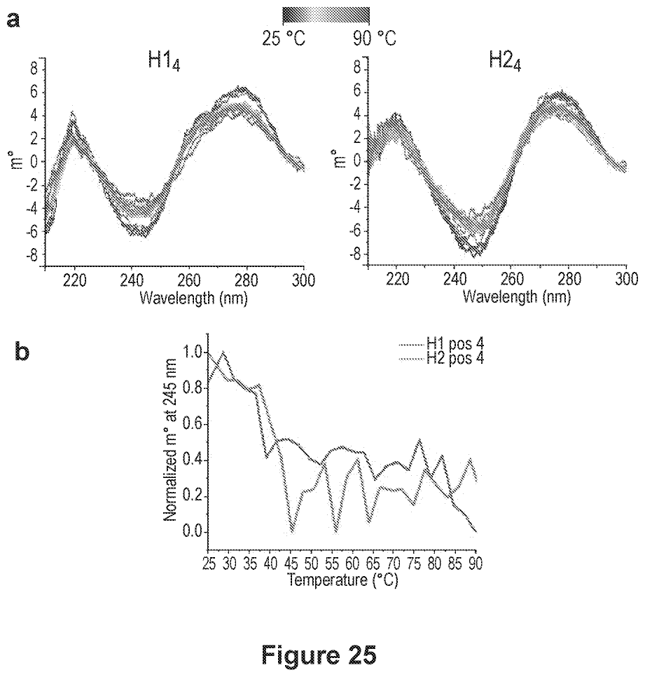

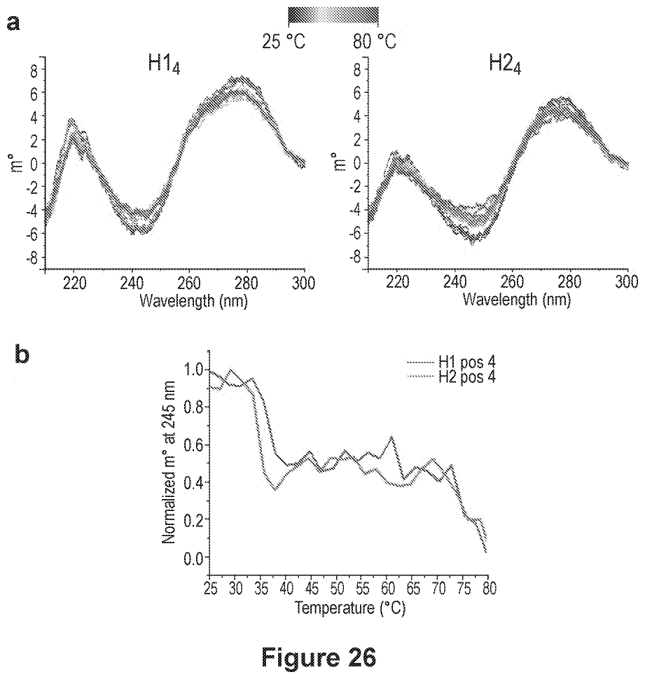

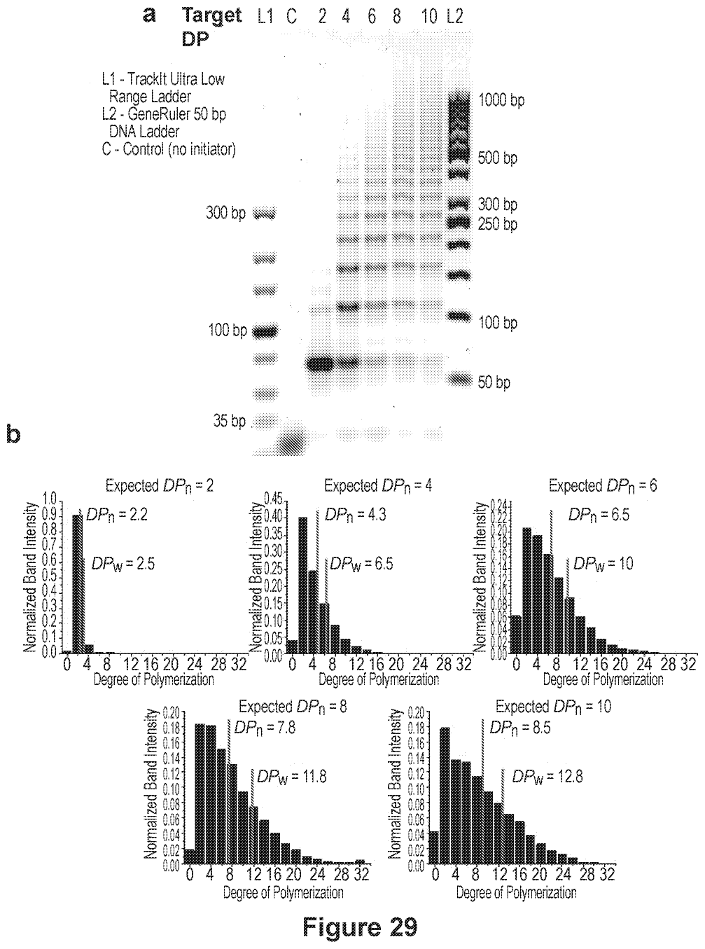

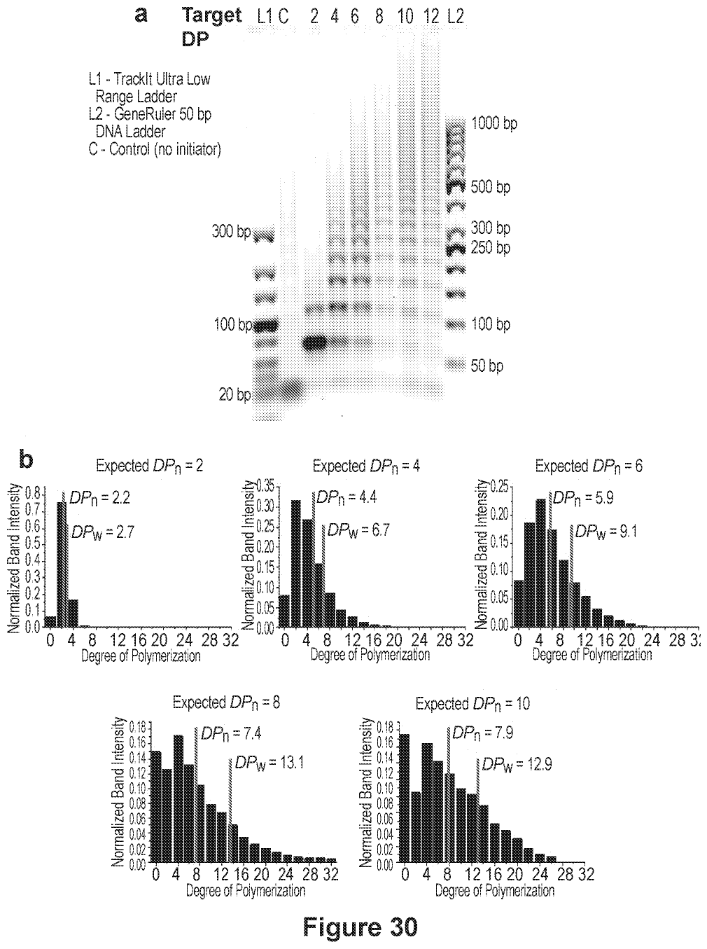

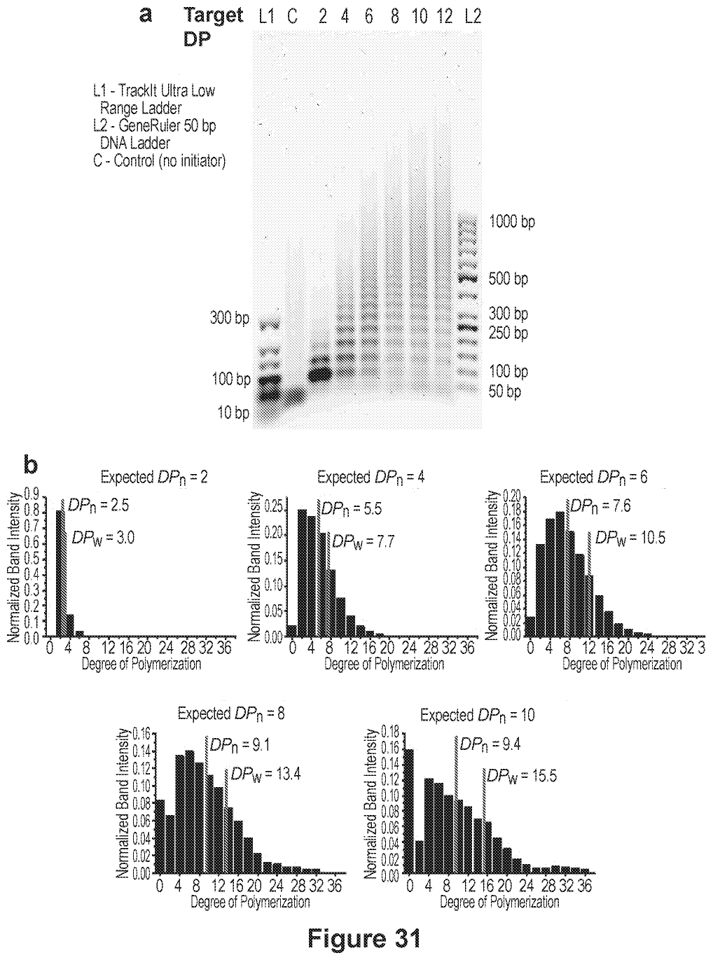

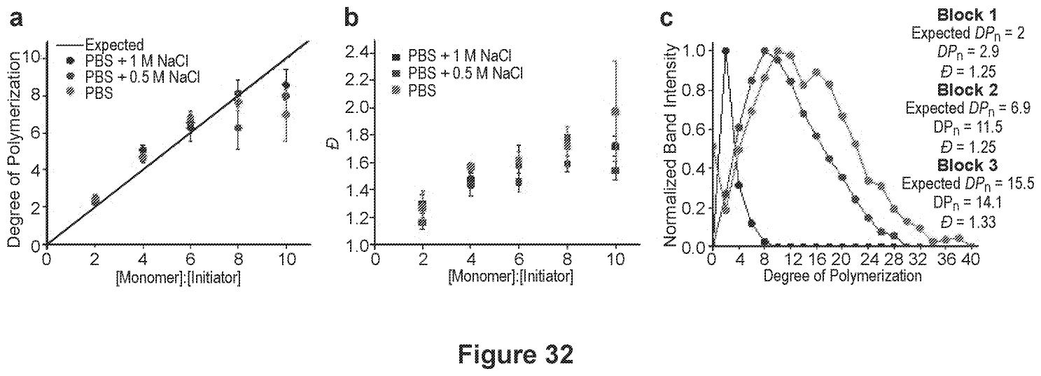

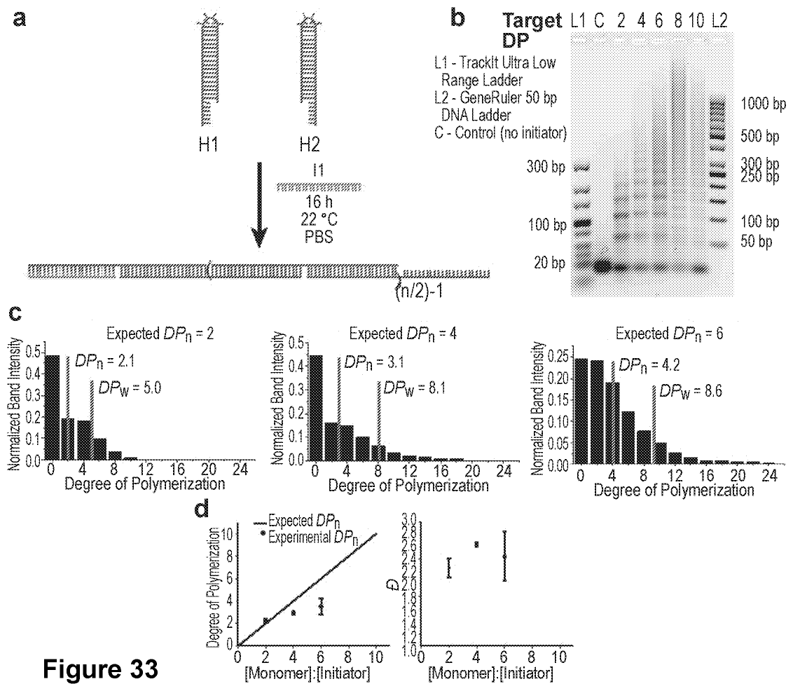

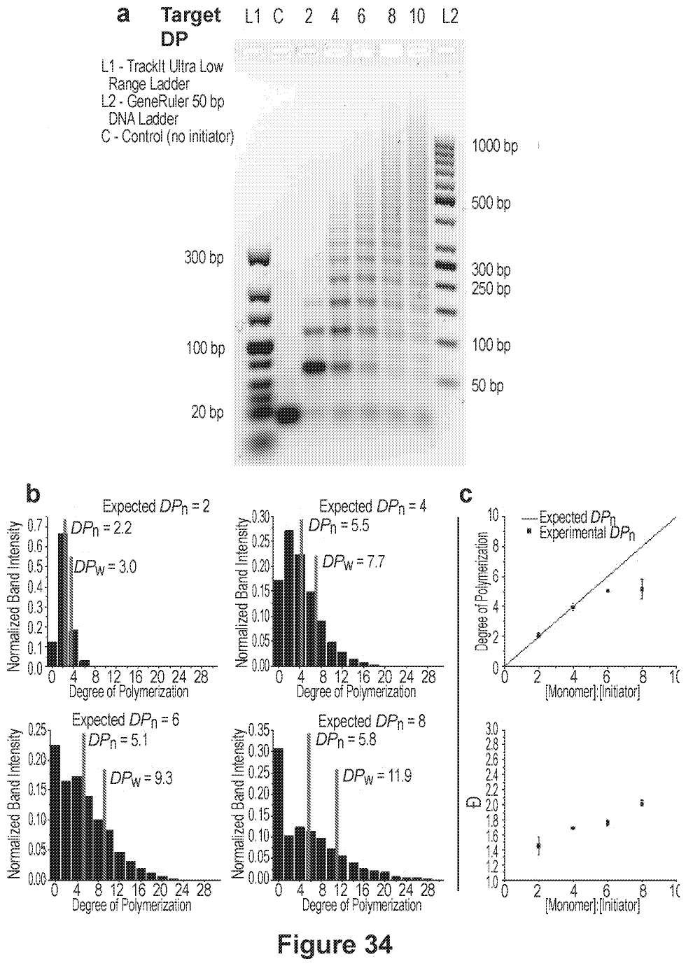

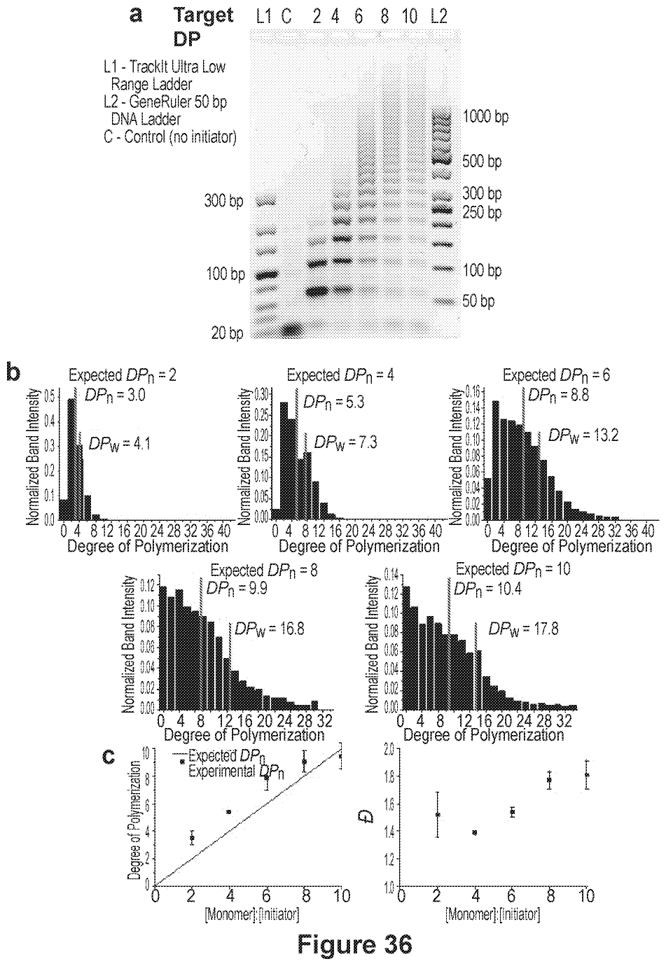

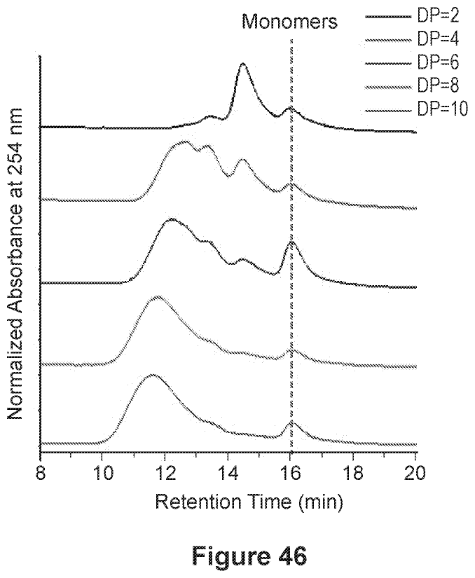

Next, to understand if the mismatch yielded oligomers with predictable DPₙ and low dispersity, reactions were set up targeting DPₙ values of 2, 4, 6, 8, and 10 by varying the H1₄ and H2₄ to I2₄ ratios in solutions at different ionic strengths (PBS, PBS+0.5 M NaCl, and PBS+1 M NaCl) to exclude any deleterious charge-charge repulsion effects (FIGS. 29-31 and FIG. 46). Experimental DPₙ values closely followed expected values up to a DPₙ=10 (FIG. 32a), above which a decrease in gel resolution inhibited analysis. Oligomer Ð=1.3-1.6 (FIG. 32b), further showing that the oligomerization was yielding low dispersity products. Additionally, higher ionic strength required heating to 37° C. due to the increased melting temperature of the bases between the toehold and mismatch (FIGS. 25 and 26), but yielded higher oligomerization conversions than PBS when targeting a DPₙ of 8 or 10. Importantly, the experimental DPs of the mismatched system agreed with the monomer:initiatior feed ratio significantly better, and lower dispersities were achieved compared to the conventional monomer system or other mismatch positions (FIGS. 33-36).

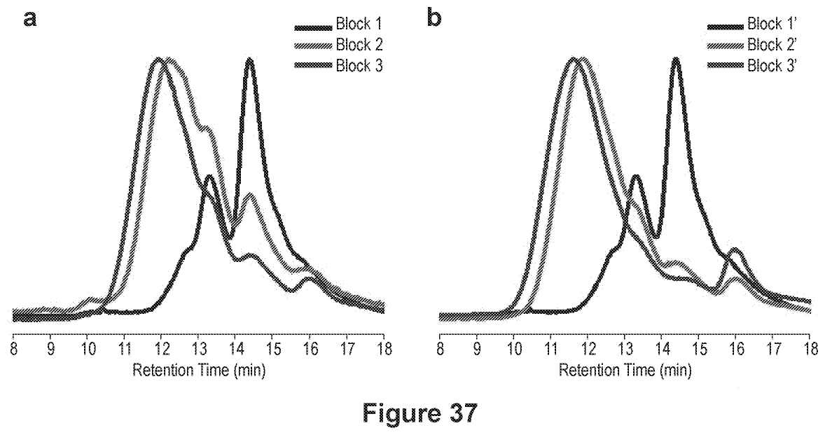

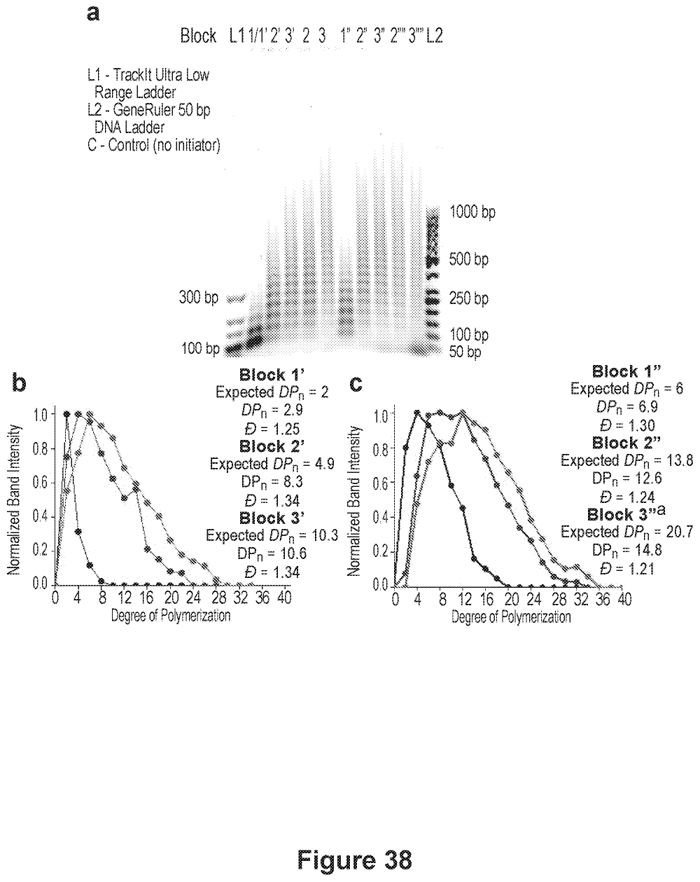

To determine if block co-oligomers with low dispersity and predictable DPₙ can be realized, a chain extension oligomerization was performed at 37° C. in PBS+1 M NaCl (FIG. 32c). DPₙ was observed to shift according to monomer additions and a final experimental DPₙ=14.1 was the highest we have observed using our controlled HCR technique, agreeing with the expected DPₙ=15.5 and showing a Ð=1.33. Other chain extensions also exhibited efficient oligomerization (FIGS. 37 and 38). This confirmed that predictable DPₙ and low dispersity are inherent to these HCR oligomerizations and are retained after the addition of fresh monomer due to the designed DNA monomers.

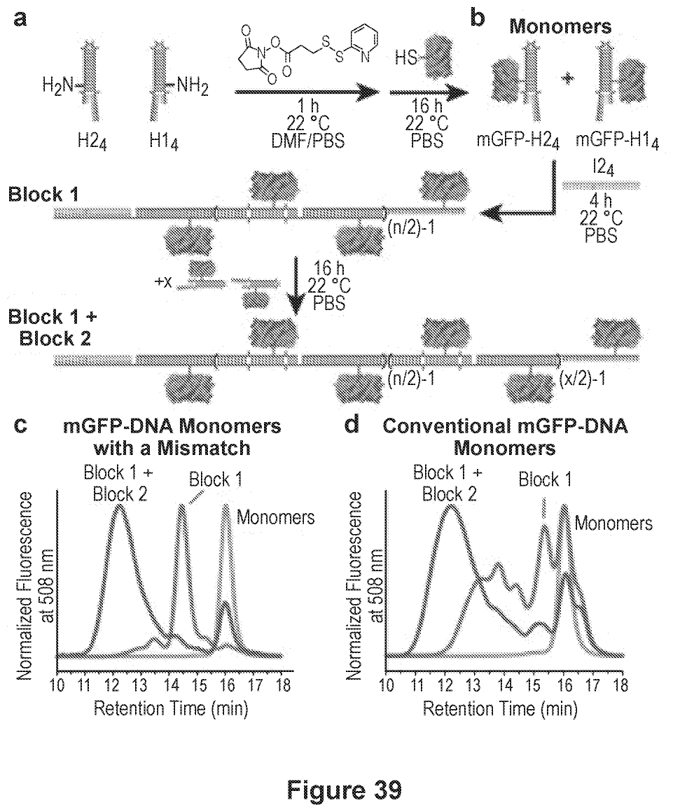

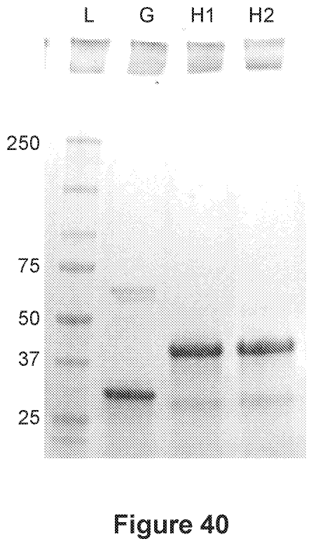

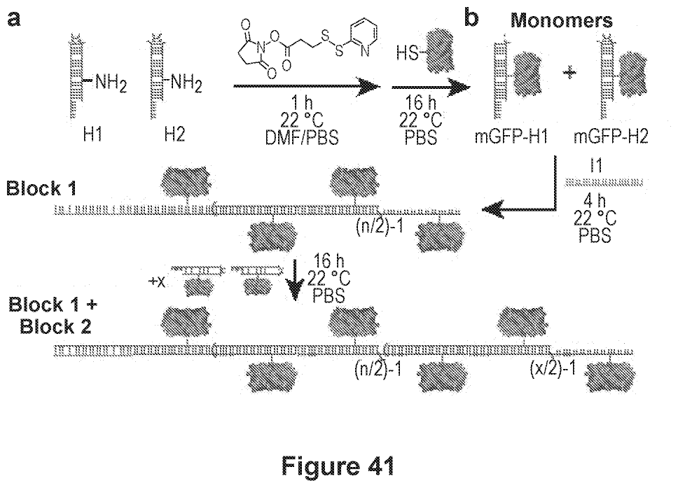

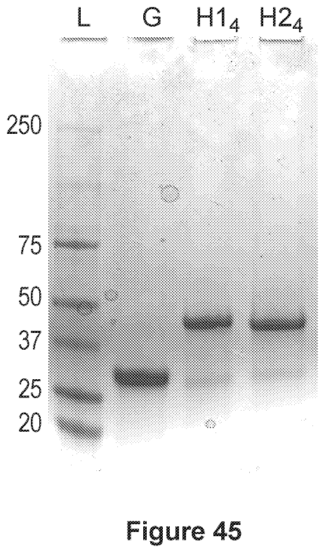

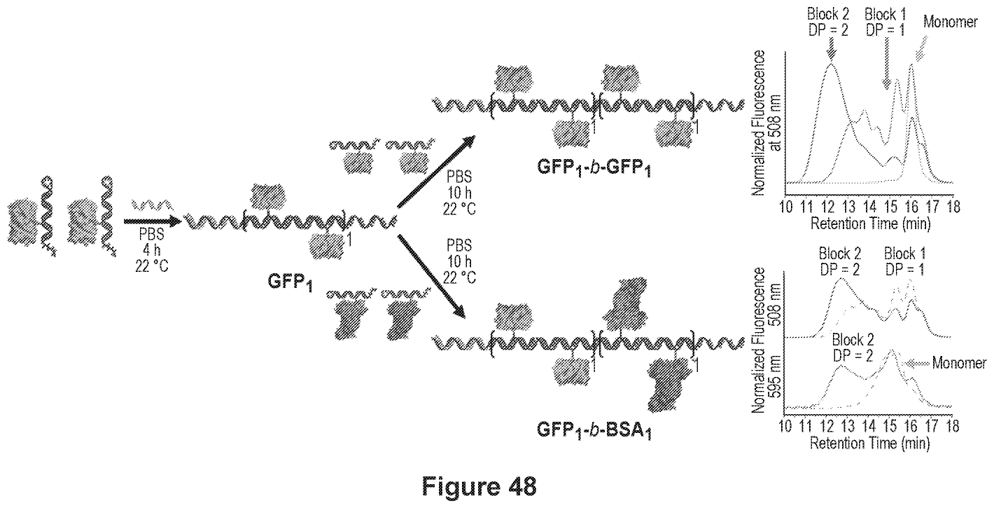

Finally, to evaluate whether the oligomerization control is retained when nanoscale cargo such as proteins are attached to the DNA hairpins [McMillan et al., J. Am. Chem. Soc. 2018, 140, 15950-15956], the mismatch hairpins H1₄ and H2₄ were conjugated to a mutant green fluorescent protein (mGFP) containing a single surface cysteine to attain mGFP-H1₄ and mGFP-H2₄ (FIGS. 39a and 45) [McMillan et al., J. Am. Chem. Soc. 2018, 140, 15950-15956; Hayes et al., J. Am. Chem. Soc. 2018, 140, 9269-9274; Brodin et al., Proc. Natl. Acad. Sci. U.S.A. 2015, 112, 4564-4569]. Oligomerization of mGFP-DNA monomers with a mismatch using I2₄ (FIG. 39b) led to analogous control over the product distributions as the DNA only system, as evidenced by SEC (FIG. 39c). Oligo(mGFP) was successfully quantitatively chain-extended, confirming that the controlled DNA oligomerization technique can be used to oligomerize proteins into low dispersity assemblies. Importantly, analogous reactions using conventional mGFP-DNA monomers (FIGS. 40 and 41) exhibited high polydispersity (FIG. 39d and FIG. 48).

In conclusion, these results showed that a strategically placed base-pair mismatch in DNA HCR monomers can be used to control DNA oligomerization, overcoming compromises between extensive DNA design and well-defined materials commonly encountered when synthesizing DNA oligomers. Additionally, introducing HCR as a controlled living oligomerization technique has significant implications for nearly every HCR application where deliberate control over DNA oligomerization will provide more well-defined materials and increased quantitative capabilities in the context of amplified detection systems.

### Materials and Methods

DNA Sequences. This information is provided in Table 6.

TABLE 6
DNA Sequences, Molecular Weights, and Extinction Coefficients.

| | | | MW | MW | |
|---|---|---|---|---|---|
| Name | SEQ ID NO: | Sequence (5′→3′) | Expected | Observed | ε₂₆₀ (M⁻¹CM⁻¹) |
| I1 | 11 | AGT CTA GGA TTC GGC GTG | 7464 | 7541 | 239600 |
| | | GGT TAA | | | |
| H1 | 1 | TTA ACC CAC GCC GAA TCC | 14737 | 14836 | 463800 |
| | | TAG ACT CAA AGT AGT CTA | | | |
| | | GGA [symbol] TC GGC GTG | | | |
| I2 | 12 | CAA AGT AGT CTA GGA TTC | 7432 | 7553 | 238700 |
| | | GGC GTG | | | |
| H2 | 2 | AGT CTA GGA T [symbol] C GGC GTG | 14799 | 14827 | 461500 |
| | | GGT TAA CAC GCC GAA TCC | | | |
| | | TAG ACT ACT TTG | | | |
| H1₂ | 3 | TTA ACC CAC GCC GAA TCC | 14745 | 14614 | 468300 |
| | | TAG ACT CAA AGT AGT CTA | | | |
| | | GGA TTC GGC G [symbol] G | | | |
| I2₂ | 14 | CAA AGT A [symbol] T CTA GGA TTC | 7393 | 7431 | 238700 |
| | | GGC GTG | | | |
| H2₂ | 15 | AGT CTA GGA TTC GGC GTG | 14838 | 14934 | 457000 |
| | | GGT TAA CAC GCC GAA TCC | | | |
| | | TAG A [symbol] T ACT TTG | | | |
| H1₄ | 16 | TTA ACC CAC GCC GAA TCC | 14786 | 14858 | 471200 |
| | | TAG ACT CAA AGT AGT CTA | | | |
| | | GGA [symbol] TC GG [symbol] GTG | | | |
| I2₄ | 13 | CAA AGT AGT [symbol] TA GGA TTC | 7473 | 7536 | 242200 |
| | | GGC GTG | | | |
| H2₄ | 6 | AGT CTA GGA T [symbol] C GGC GTG | 14759 | 14808 | 457800 |
| | | GGT TAA CAC GCC GAA TCC | | | |
| | | TA [symbol] ACT ACT TTG | | | |
| H1₆ | 17 | TTA ACC CAC GCC GAA TCC | 14706 | 14641 | 464800 |
| | | TAG ACT CAA AGT AGT CTA | | | |
| | | GGA TTC [symbol] GC GTG | | | |
| I2₆ | 18 | CAA AGT AGT CT [symbol] GGA TTC | 7424 | 7448 | 238700 |
| | | GGC GTG | | | |
| H2₆ | 19 | AGT CTA GGA TTC GGC GTG | 14807 | 14865 | 455600 |
| | | GGT TAA CAC GCC GAA TCC | | | |
| | | [symbol] AG ACT ACT TTG | | | |
| H1₈ | 9 | TTA ACC CAC GCC GAA TCC | 14746 | 14675 | 468700 |
| | | TAG ACT CAA AGT AGT CTA | | | |
| | | GGA T [symbol] C GGC GTG | | | |
| I2₈ | 20 | CAA AGT AGT CTA G [symbol] A TTC | 7393 | 7316 | 238700 |
| | | GGC GTG | | | |
| H2₈ | 21 | AGT CTA GGA TTC GGC GTG | 14838 | 14917 | 457600 |
| | | GGT TAA CAC GCC GAA T [symbol] C | | | |
| | | TAG ACT ACT TTG | | | |
| I1- | 22 | BHQ AGT CTA GGA TTC GGC | 8003 | 8006 | 239600 |
| BHQ | | GTG GGT TAA | | | |
| H1- | 23 | TTA ACC CAC GCC GAA TCC | 14954 | 15006 | 462100 |
| CY3 | | TAG ACT CY3 AA AGT AGT CTA | | | |
| | | GGA [symbol] TC GGC GTG | | | |
| I2₄- | 24 | CAA AGT AGT [symbol] TA GGA TTC | 8028 | 8032 | 242200 |
| BHQ | | GGC GTG BHQ | | | |
| P2₄- | 25 | CAA AGT AGT CTA GGA TTC | 8028 | 8112 | 242200 |
| BHQ | | GG [symbol] GTG BHQ | | | |
| H2₄- | 26 | AGT CTA GGA TTC GGC GTG | 14952 | 14988 | 450700 |
| CY3 | | GGT TA CY3 CAC GCC GAA TCC | | | |
| | | TA [symbol] ACT ACT TTG | | | |

[symbol]

- denotes mismatch position, [symbol]
- denotes where T was substituted with amino dT modifier (Glen Research) for sequences designed for mGFP-DNA conjugates.

DNA Synthesis. Oligonucleotides utilized herein were synthesized on solid supports using reagents obtained from Glen Research and standard protocols. Products were cleaved from the solid support using 15% (w/v) NH₃OH (aq) and 20% (w/v) CH₃NH₂ for 20 min at 55° C. and purified using reverse-phase HPLC with a gradient of 0 to 75 percent acetonitrile in triethylammonium acetate buffer over 45 minutes. Dimethoxytrityl or monomethoxytrityl groups were cleaved with 20% (v/v) acetic acid for 2 h and extracted with ethyl acetate. The masses of the oligonucleotides were confirmed using matrix-assisted laser desorption ionization mass spectrometry (MALDI-MS) using 3-hydroxypicolinic acid, 2′,5′-dihydroxyacetophenone, or 2′,4′,6′-trihydroxyacetophenone monohydrate as a matrix. All synthesized DNA masses were within 100 Da of the expected mass.

DNA Monomer Preparation. Following DNA purification, each monomer was diluted in PBS with the appropriate added salt concentration (either 0, additional 0.5 M NaCl, or additional 1 M NaCl) to a concentration of 20 μM. The solution was then snapcooled by heating to 95° C. for 4 min, cooled to 4° C. over 1 minute, held at 4° C. for 4 min, then brought to room temperature in an Applied Biosystems SimpliAmp Thermal Cycler. The concentration was then confirmed using the DNA extinction coefficient at 260 nm.

DNA Oligomerizations at 22° C. Oligomerizations were conducted at 5 μM per monomer. For example, for an oligomerization targeting a DP=4, 13.5 μL of 18.4 μM H1_{4, 13.2} L of 18.9 μM H2₄, and 20.9 μL PBS were added to an Eppendorf tube. Then, 2.51 μL of 49.2 μM I2₄ was added and the polymerization was allowed to react for 16 h before an aliquot was taken for agarose gel electrophoresis analysis. Each monomer to initiator feed ratio was repeated 3× to determine average DP.

DNA Oligomerization at 37° C. Polymerizations were conducted at 5 μM per monomer. For example, for an oligomerization targeting a DP=4, 13.5 μL of 18.4 μM H1_{4, 13.2} L of 18.9 μM H2₄, and 20.9 μL PBS+1 M NaCl were added to an Eppendorf tube. H1₄ and H2₄ were equilibrated for 10 minutes at 37° C. Then, 2.51 μL of 49.2 μM I2₄ was added and the polymerization was allowed to react for 16 h before an aliquot was taken for agarose gel electrophoresis analysis. Each monomer to initiator feed ratio was repeated 3× to determine average DP.

DNA Oligomer Chain Extensions.

Block 1—Polymerizations were conducted at 5 μM per monomer. For a polymerization targeting a first block of DP=2, 5.95 μL of 16.8 μM H1₄, 6.94 μL of 14.4 μM H2₄, and 5.11 μL PBS+1 M NaCl were added to an Eppendorf tube. H1₄ and H2₄ were equilibrated for 10 minutes at 37° C. Then, 2.04 μL of 49.2 μM I2₄ was added and the polymerization was allowed to react for 16 h. Experimental DPₙ=2.9, [symbol] =1.25 via agarose gel electrophoresis analysis.

Block 2—First, 5 μL of Block 1 were added to an Eppendorf tube. Then, to target a second block DPₙ=4.9, 1.49 μL of 16.8 μM H1₄ and 1.74 μL of 14.4 μL H2₄ were added and the reaction was set at 37° C. for 24 h (expected DPₙ was calculated from the experimental DP of Block 1). Experimental DPₙ=8.3, [symbol] =1.34 via agarose gel electrophoresis analysis.

Block 3—Next, 5 μL of Block 2 were added to a new Eppendorf Tube. To target a second block DP=10.3, 0.745 μL of 16.8 μM H1₄ and 0.87 μL of 14.4 μL H2₄ were added and the reaction was set at 37° C. for 24 h (expected DPₙ was calculated from the experimental DP of Block 2). Experimental DPₙ=10.6, [symbol] =1.34 via agarose gel electrophoresis analysis.

mGFP-DNA Conjugate Synthesis. Example mGFP-H1₄ synthesis—Succinimidyl 3-(2-pyridylithio)propionate (5 mg, 15.5 μmol, SPDP, Thermo Scientific) was dissolved in 500 μL DMF in an Eppendorf tube. H1₄ (315 μL, 983 μM, 310 nmol in PBS) and 185 μL PBS were then added. After 1 h at 22° C., the reaction was purified via size-exclusion chromatography using a NAP25 column (GE Healthcare) equilibrated in PBS. The solution was then snapcooled by heating to 95° C. for 4 min, cooled to 4° C. over 1 minute, held at 4° C. for 4 min, then brought to room temperature. Subsequently, purified mutated green fluorescent protein (608 μL, 102 μM, 62 nmol, mGFP, expressed and purified according to previous procedures [Hayes et al., DNA-Encoded Protein Janus Nanoparticles. J. Am. Chem. Soc. 2018, 140, 9269-9274; McMillan et al., J. Am. Chem. Soc. 2018, 140, 15950-15956]) was added and the solution was set at 22° C. for 16 h. To purify the mGFP-H1₄ conjugate, the solution was loaded onto a Profinity IMAC column (Bio-Rad), washed with 20 mL PBS to remove excess DNA, then 10 mL PBS+250 mM imidazole to elute the protein. Next, the eluent was loaded onto a Macro-Prep DEAE resin (Bio-Rad) and washed with 10 mL PBS, 20 mL PBS+0.35 M NaCl to elute mGFP, then PBS+0.5 M NaCl to elute mGFP—H1₄.

Agarose Gel Electrophoresis. Agarose (Sigma Aldrich) was added to 40 mL 1×TBE at the desired weight concentration (1-3 w/w %). The flask was microwaved to completely dissolve the agarose, 1 μL GelRed (10000×, Biotium Inc.) was added, and the gel was set at room temperature. DNA samples were prepared by mixing 8 μL PBS, 2 μL oligomerization sample, and 1 μL TrackIt Cyan/Yellow Loading Dye (6×, Invitrogen). Both TrackIt Ultra Low Range DNA Ladder (Invitrogen) and GeneRuler 50 bp DNA Ladder (Invitrogen) were used as standards. Gels were run for 2.25 h at 100V and imaged using a Bio-Rad ChemiDoc MP Imaging System using the GelRed scanning settings.

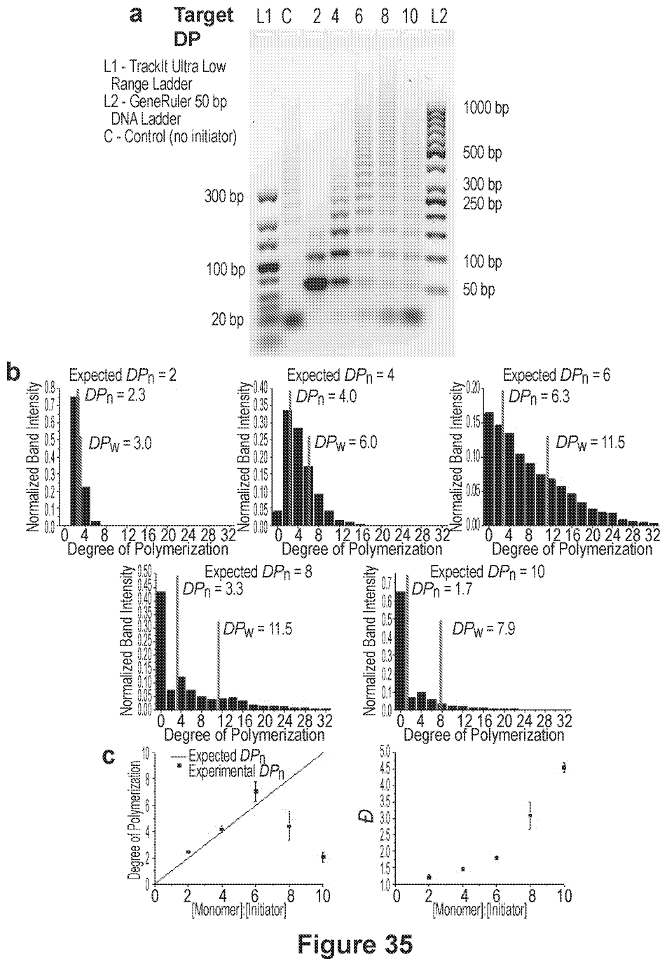

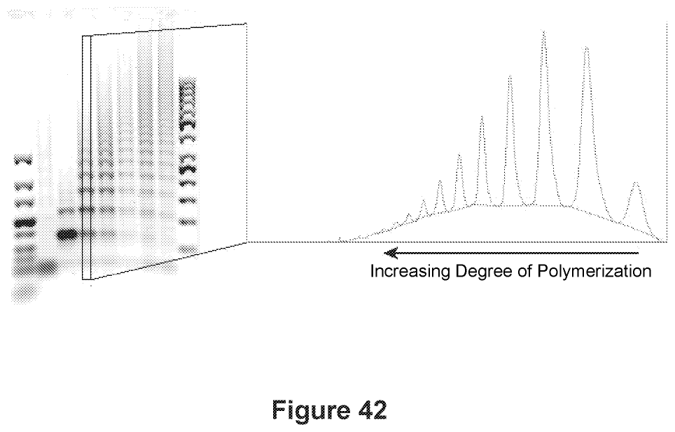

To analyze oligomerization, ImageJ software was used to plot the gel band intensity (See FIG. 42, which used the agarose gel from FIG. 35) as an example):

The area under each band peak was calculated according to arbitrary values assigned by ImageJ software, and each degree of polymerization was normalized to total area calculated and calculations were based on the following equations to determine number-average degree of polymerization (DPₙ, Equation 1), weight-average degree of polymerization (DP_{w}, Equation 2), and dispersity ( [symbol] , Equation 3).

Each gel was analyzed individually, then the DPₙ, DP_{w}, and Ð corresponding to the same conditions were averaged and standard error was calculated to determine final values.

Size-Exclusion Chromatography (SEC). SEC was performed using an Agilent 1260 Infinity System HPLC operating at 22° C. equipped with Viscotek 6000M General Mixed column and Agilent Advance Bio SEC 300 Å columns run in sequence using a mobile phase of PBS at a flow rate of 1 mL/min. DNA elution profiles were monitored at an absorbance wavelength of 254 nm. For GFP SEC analysis, elution profiles were monitored using a fluorescence detector with an excitation at 488 nm and emission detection at 509 nm.

Circular Dichroism Measurements. CD measurements were conducted on a Jasco J-1700 using a DNA concentration of 10 μM in either PBS, PBS+0.5 M NaCl, or PBS+1 M NaCl. Melting temperature was determined by taking 3 CD measurements every 2° C. from 25-90° C. at a temperature ramp rate of 0.1° C./min, averaging the curves, normalizing the m° at 275 nm, and taking the temperature at the 0.5 y-axis value after normalization.

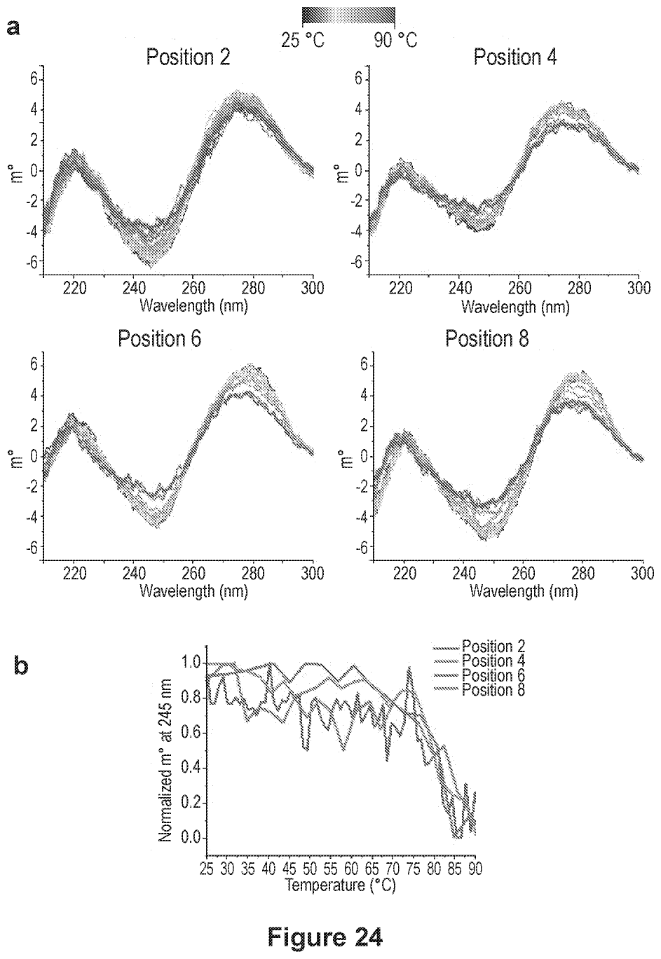

Melting experiments of the hairpins using circular dichroism showed that hairpin duplex stability was minimally affected, with melting temperatures all between 60-80° C. (Table 5, FIG. 23, and FIG. 24). Additionally, increasing salt concentrations to favor DNA duplex formation (FIG. 25 and FIG. 26) showed a melting transition emerge at 35-40° C., corresponding to the fraying of the stem between mismatch and toehold, and a second transition >75° C. for the full melting of the hairpin for H1₄ and H2₄, suggesting that increased fraying of the bps between the toehold and the mismatch was occurring, effectively increasing the length of the toehold. However, the fraying was not long enough to cause background oligomerization in the absence of initiator during the dimerizations (FIG. 19 and FIG. 20).

Fluorescence/Quencher Kinetic Measurements. Measurements were taken on a Biotek Cytation 5 Cell Imaging Multi-Mode Reader equipped with an autoinjector. Reactions were performed at different hairpin concentrations (20, 30, 40, and 50 nM, FIG. 28a-c) in a solution of 5 μM I1-BHQ, I2₄-BHQ, or P2₄-BHQ to achieve pseudo-first order kinetics conditions. As an example, for reactions measuring kinetics of H2₄-Cy3 opening by I2₄-BHQ at a concentration of 20 nM, 50 μL of a 20 μM I2₄-BHQ solution was diluted in PBS (130 μL) in a 96-well plate. 20 μL of a solution of 200 nM H2₄-Cy3 was injected using the autoinjector, and readings were taken every second immediately following injection for 120 s. Each measurement was taken 3 times and averaged for the concentration data plots (FIG. 28). To normalize the data, the maximum fluorescence used was the average of a hairpin fluorescence measurement without quencher to account for any photobleaching. The maximum possible quenching was set at the quenching measured from a thermally annealed hairpin-initiator duplex after a slow cool from 95-25° C. at a cooling rate of 1° C./min. Excess of the BHQ-strand was used to achieved pseudo-first order conditions. An initial decrease in fluorescence faster than could be measured was predominately attributed to toehold binding, as BHQ would be within the reported 50% quenching of the Cy3-BHQ dye pair [Chen et al., J. Am. Chem. Soc. 2012, 134, 5734-5737]. The subsequent decrease in fluorescence was attributed to the full strand displacement reaction. Therefore, the collected data was fit to a first-order decay rate equation. To calculate the observed strand-displacement rate (k_{obs}), each measurement was individually fit using the first-order exponential decay equation in OriginPro 2020 software, then averaged to calculate k_{obs}. Any higher-order fitting equations or artificially including the t=0 point yielded poor fits with lower R² values, but similar rate constants as the first-order decay fitting. I1-Cy3 and P2₄-Cy3 reaction curves were fit for 120 s of measurements. I2₄ was fit for 30 s all yielding values of R²>0.97. Because first-order decay fitting was found to be concentration-independent (FIG. 28d), this final decay was taken as k_{obs}. Both mismatch systems showed a larger decrease in initial fluorescence which, in combination with hairpin melting experiments (FIGS. 25 and 26), suggested an increased toehold length.

What is claimed is:

1. A method of producing a structure, the method comprising contacting:
a) a first oligonucleotide hairpin monomer comprising a first toehold, a first duplex stem, and a first hairpin loop,
wherein the first duplex stem comprises a base-pair mismatch at a position that is proximal to the first toehold and distal to the first hairpin loop, relative to a midpoint of the first duplex stem;
b) a second oligonucleotide hairpin monomer comprising a second toehold, a second duplex stem, and a second hairpin loop; and
c) an initiator oligonucleotide,
wherein hybridization of the initiator oligonucleotide to the first oligonucleotide hairpin monomer results in hybridization of the first oligonucleotide hairpin monomer to the second oligonucleotide hairpin monomer, thereby producing the structure.

2. The method of claim 1, wherein the base-pair mismatch in the first duplex stem is located at a position that is 1, 2, 3, 4, 5, 6, 7, 8, 9, 10, 11, 12, 13, 14, or 15 nucleotides from the first toehold.

3. The method of claim 1 or claim 2, wherein the first duplex stem comprises 2, 3, 4, or 5 base-pair mismatches.

4. The method of any one of claims 1-3, wherein the second duplex stem comprises a base-pair mismatch at a position that is proximal to the second toehold and distal to the second hairpin loop, relative to a midpoint of the second duplex stem.

5. The method of claim 4, wherein the base-pair mismatch in the second duplex stem is located at a position that is 1, 2, 3, 4, 5, 6, 7, 8, 9, 10, 11, 12, 13, 14, or 15 nucleotides from the second toehold.

6. The method of claim 4 or claim 5, wherein the second duplex stem comprises 2, 3, 4, or 5 base-pair mismatches.

7. The method of any one of claims 1-6, wherein the first duplex stem comprises from about 10 to about 50 nucleotides.

8. The method of any one of claims 1-7, wherein the second duplex stem comprises from about 10 to about 50 nucleotides.

9. The method of any one of claims 1-8, wherein the first hairpin loop comprises from about 3 to about 30 nucleotides.

10. The method of any one of claims 1-9, wherein the second hairpin loop comprises from about 3 to about 30 nucleotides.

11. The method of any one of claims 1-10, wherein the toehold of the first oligonucleotide hairpin monomer comprises from about 1 to about 50 nucleotides.

12. The method of any one of claims 1-11, wherein the toehold of the second oligonucleotide hairpin monomer comprises from about 1 to about 50 nucleotides.

13. The method of any one of claims 1-12, wherein the second hairpin loop and the first toehold are about 70%, about 80%, about 90%, about 99%, or 100% complementary to each other.

14. The method of any one of claims 1-13, wherein the second toehold and the first hairpin loop are about 70%, about 80%, about 90%, about 99%, or 100% complementary to each other.

15. The method of any one of claims 1-14, wherein the initiator oligonucleotide is an analyte to be detected in a sample.

16. The method of any one of claims 1-15, wherein the initiator oligonucleotide is single stranded.

17. The method of any one of claims 1-16, wherein the initiator oligonucleotide is messenger RNA (mRNA).

18. The method of any one of claims 1-17, wherein the first hairpin oligonucleotide monomer further comprises an additional moiety.

19. The method of any one of claims 1-18, wherein the second oligonucleotide hairpin monomer further comprises an additional moiety.

20. The method of any one of claims 1-19, wherein the initiator oligonucleotide further comprises an additional moiety.

21. The method of any one of claims 18-20, wherein the additional moiety is an aptamer, protein, a peptide, a nanoparticle, a small molecule a quantum dot, a detectable marker, or a combination thereof.

22. The method of claim 21, wherein the protein is an antibody.

23. The method of claim 22, wherein the first oligonucleotide hairpin monomer further comprises an antibody and the second oligonucleotide hairpin monomer further comprises an additional antibody.

24. The method of claim 23, wherein the antibody and the additional antibody are different.

25. The method of claim 21, wherein the detectable marker is a fluorophore.

26. The method of any one of claims 1-25, wherein a plurality of first oligonucleotide hairpin monomers, initiator oligonucleotides, and second oligonucleotide hairpin monomers are contacted.

27. The method of any one of claims 1-26, wherein the ratio of the first oligonucleotide hairpin monomer to the initiator oligonucleotide is about 1:1, 2:1, 4:1, 6:1, 8:1, or 10:1.

28. The method of any one of claims 1-27, wherein the ratio of the second oligonucleotide hairpin monomer to the initiator oligonucleotide is about 1:1, 2:1, 4:1, 6:1, 8:1, or 10:1.

29. The method of any one of claims 1-28, wherein the structure is detected by gel electrophoresis, mass spectrometry, light scattering spectroscopy, colorimetry, fluorescent microscopy, fluorescent spectroscopy, electron microscopy, atomic force microscopy, nuclear magnetic resonance (NMR) depending or a combination thereof.

30. The method of any one of claims 1-29, wherein the first oligonucleotide hairpin monomer is DNA, RNA, or a modified form thereof.

31. The method of any one of claims 1-30, wherein the second oligonucleotide hairpin monomer is DNA, RNA, or a modified form thereof.

32. The method of any one of claims 1-31, wherein the initiator oligonucleotide is DNA, RNA, or a modified form thereof.

33. The method of any one of claims 1-32, further comprising contacting the structure with a termination oligonucleotide.

34. A method of detecting an analyte in a sample, the method comprising contacting the sample with:
a) a first oligonucleotide hairpin monomer comprising a first toehold, a first duplex stem, a first hairpin loop, and a fluorescent marker,
wherein the first duplex stem comprises a base-pair mismatch at a position that is proximal to the first toehold and distal to the first hairpin loop, relative to a midpoint of the first duplex stem;
b) an initiator oligonucleotide; and
c) a second oligonucleotide hairpin monomer comprising a second toehold, a second duplex stem, and a second hairpin loop;
wherein one or more of the first duplex stem, the first toehold, and the initiator oligonucleotide comprises an analyte-binding region; and
measuring fluorescence of the sample after the contacting to detect the analyte.

35. The method of claim 34, wherein the analyte is a nucleic acid.

36. The method of claim 34 or claim 35, wherein the analyte is DNA, RNA, a protein, a peptide, or a combination thereof.

37. The method of claim 36, wherein the analyte is messenger RNA (mRNA), microRNA (miRNA), small interfering RNA (siRNA), or a combination thereof.

38. The method of any one of claims 35-37, wherein the analyte-binding region is substantially complementary to the nucleic acid.

39. The method of any one of claims 34-38, wherein the second oligonucleotide hairpin monomer comprises an additional fluorescent marker.

40. The method of claim 39, wherein the fluorescent marker and the additional fluorescent marker are different.

41. The method of any one of claims 34-40, wherein the analyte-binding region comprises an antibody.

42. The method of any one of claims 34-41, wherein the sample is contacted with a plurality of first oligonucleotide hairpin monomers, initiator oligonucleotides, and/or second oligonucleotide hairpin monomers.
